(19) United States
(12) Patent Application Publication
(10) Pub. No.: US 2016/0060658 A1
(43) Pub. Date: Mar. 3, 2016

(54) FERMENTATION SYSTEM FOR DRY MILL PROCESSES

(71) Applicant: Lee Tech LLC., San Jose, CA (US)

(72) Inventor: Chie Ying Lee, San Jose, CA (US)

(21) Appl. No.: 14/839,763

(22) Filed: Aug. 28, 2015

Related U.S. Application Data
(60) Provisional application No. 62/044,092, filed on Aug. 29, 2014.

(51) Int. Cl. C12P 7/06; C12N 1/16

(52) U.S. Cl. CPC C12P 7/06; C12N 1/16

(57) ABSTRACT

Methods of and system for growing higher and stronger levels of yeast in the Yeast Tank and Fermenter Tank during the fermentation filling cycle are provided. The yeast growth is reconfigured by continuing pumping yeast under the most active conditions while at a lower YCC (yeast cell count) into the Fermenter Tank during a filling period. A measurable and useful parameter, % DT/% Yeast by weight ratio (or “food” to yeast ratio), is introduced (e.g., % DT=glucose), which offers information on the health status of the yeast.

### CROSS-REFERENCE TO RELATED APPLICATION(S)

This application claims priority under 35 U.S.C. §119(e) of the U.S. Provisional Patent Application Ser. No. 62/044,092, filed Aug. 29, 2014 and titled, “NEW IMPROVEMENT FERMENTATION SYSTEM FOR DRY MILL PROCESS,” which is hereby incorporated by reference in its entirety for all purposes.

### FIELD OF THE INVENTION

The present invention relates to the field of fermentation. More specifically, the present invention relates to growing higher and stronger levels of yeast in the Yeast Tank and Fermenter Tank during a fermentation filling cycle.

### BACKGROUND OF THE INVENTION

As the world's population continues to grow, fossil fuel resources such as gasoline are going to be consumed and depleted. Scientists have researched and developed using ethanol, a two-carbon alcohol compound, as an effective additive to gasoline to curb the rapid usage of gasoline. In some cases, gasoline mixtures have as high as 85% volume of ethanol as a biofuel. Although coal and oil produce carbon dioxide from previously long-term sequestered carbon, the carbon dioxide produce from the combustion of grain alcohol is consumed by growing the grain and quickly recycled in the environment resulting in no net carbon dioxide addition to the atmosphere, thus not leading to greenhouse gas accumulation. In the following, few typical alcohol producing systems using a typical dry mill is discussed.

FIG. 1 shows an overview of a typical dry grind process. At a Step 10, raw grain carbohydrate is procured and set as a starting feed. At a Step 11, the grain carbohydrate feed is sent to a particle size reduction step, commonly hammer mill or roller mill, to shred, crush, and pulverize the grain carbohydrate into smaller pieces.

At a Step 12, the broken up grain carbohydrate is sent to a tank where water stream(s) of various quality are added in a controlled ratio to produce a grain slurry. This slurry can be produced using heated water streams which gelatinize a significant fraction of the starch in the grain carbohydrate. This process is often referred to as the hot cook process. This slurry can alternately be produced using water streams which temperature, when combined with the grain carbohydrate, are below the gelatinization temperature of a significant fraction of the starch in the grain carbohydrate. This process is often referred to as the raw starch process. Enzyme or enzyme mixtures are often added into the slurry to a cooked water bath to make a dextrin solution. Controlled metering of grain and water along with thorough agitation mixing ensures uniform consistency of the batch.

At a Step 13, liquefaction is performed, which is a process of changing the solid grain into liquid slurry. Liquefaction is the first step in adding water back across the bonds between sugar residues found in starch.

Starch[C₆H₁₀O₅]ₙ+H₂O(liquid)+heat→maltodextrin(liquid solution)

The maltodextrin liquid solution is a mixture of primarily soluble glucose oligomers with some residual long-chain dextrins and starch molecules that have low water solubility. The resultant dry solid of this grain starch solution ranges between 30% and 40% weight of the total output and the average dry solid is 35% weight of the total output. The typical starch content of this grain starch solution consists between 20% and 30% weight of the total output. The typical starch content is 25% weight of the total output.

At a Step 14, saccharification and fermentation are performed. Saccharification is a process breaking down complex carbohydrates into simple sugars, primarily glucose.

Dextrins[C₆H₁₀O₅]ₙ+H₂O(liquid)+glucoamylase→nC₆H₁₂O₆glucose

In the fermentation process, ethanol and carbon dioxide are produced via a biological process, where a sugar and yeast are mixed together and the sugar converted into cellular energy. The yeast metabolizes carbohydrates (primarily monosaccharides and disaccharides) to produce ethanol (liquid) and the byproduct carbon dioxide (gas). Under carefully moderated pH and temperature conditions to grow the yeast, the sugar-to-ethanol conversion can be up to 98% of theoretical maximum. Maximizing the yield and purity of ethanol quite important for commercial profitability. Fermentation is an anaerobic process that is conducted in the absence of large concentrations of oxygen.

Simple sugar(e.g. glucose)+yeast→2C₂H₅OH(ethanol)+2CO₂(carbon dioxide)

At a Step 15, distillation and dehydration are performed. The ethanol in a beer solution (alcohol, water and non-fermentable grain material) is separated into the whole stillage and ethanol through the distillation process. The whole stillage can further be refined into a byproduct, which is called distillers dried grains with soluble (DDGS), and can further recover valuable oil.

Batch fermentation plants run fermentation process flow of a 30-80 hour cycle with multiple Fermenter Tanks, commonly 3 to 8 Tanks are in facilities. Yeast can be conditioned in a yeast growth tank, often called propagation. When the yeast in the Yeast Propagation Tank has grown to a mature, healthy state, the Yeast Solution is dumped into a Fermenter Tank. Enzyme(s) is added to the fresh mash containing at least a saccharifying activity for the purpose of converting dextrins in the mash to simple sugars.

The Fermenter Tank is then filled with fresh mash over a period of from 0 to approximately 24 hours or until the Fermenter Tank is full while yeast grow and ferment sugars. The Fermenter Tank is then set idle to age allowing the yeast to continue to ferment sugars to alcohol. At the end of the cycle, the Fermenter Tank has the fermentation broth discharged and the tank cleaned to be ready for another cycle.

Empirical data and calculations presented are based on a 52 hour fermentation cycle with 4 Fermenter Tanks filled in sequence. After 13 hours of fill, the Fermenter Tank is full, disconnected from fresh mash feed, and allowed to age for the next 39 hours to complete the fermentation process. The Fermenter Tank that has completed the 52 hour cycle has the fermentation broth discharged and is set to begin a new 52 hour cycle.

FIGS. 2.1 to 2.4 shows a fermentation process with a 52 hour cycle with 4 rotating Fermenter Tanks

FIGS. 3.1A and 3.1B contain a table of simulation data of the Yeast Tank of the typical system. All the computer simulation calculations below are based on a typical 50 million gallons a year dry grind plant, which has a 15,000 gal yeast tank operation capacity, 500,000 gal fermentater operation capacity, and around 600 GPM of mash rate with a total around 13 hour filling time. In some cases, 705,000 gallon system is used. The column labeled “n” designates the time status at each hour. There is a total of 13 hours. The column labeled “Total” designates the total amount of solution in the Yeast Tank at each hour. The column labeled “y(n)” is the YCC (yeast cell count) at each hour. At hour 0, 100 pounds of dry yeast is added. The conversion factor is 2.50 pounds per dry yeast and per 15,000 gallons, which produces a YCC of 1.0. Therefore, the Yeast Tank has a YCC of 40.00 (e.g., 100/2.50). The cell count at time n is equal to the cell count value at time (n−1) plus the calculated YCC rate change, dy/dt, value at time (n−1).

$$Yeast Count = y ( n ) = y ( n - 1 ) + \frac{d y}{d t} ( n - 1 )$$

The column labeled “dy/dt” designates the YCC rate increase in the yeast tank. It will use next equation with t=n to calculate the values.

The column labeled “o(n)” designates the % Alcohol at hour n. At hour 0, the initial amount is 0.00. The alcohol at time n is equal to the alcohol (n−1) plus the calculated do/dt value at (n−1) described below.

$$\% Alcohol = o ( n ) = o ( n - 1 ) + \frac{d o}{d t} ( n - 1 )$$

The column labeled “do/dt” designates the alcohol rate increase in the yeast tank. It will use the equation with t=n to calculate the values. The column labeled “DT produced by GA” is the amount of DT (dextrose or glucose) that was additionally created by GA. At hour 0, the initial amount was 0.00. The conversion rate has been empirically set to be 0.50% DT every hour. The values from hour 1 to hour 13 are calculated by adding 0.50 after each hour.

DT produced by GA(n)=DT produced by GA(n−1)+0.50

The column labeled “DT consumed by Alcohol” is the amount of DT that is converted to Alcohol. At hour 0, the initial amount was 0.00. The conversion rate has been theoretically determined to consume around 2% DT for every 1% percent of Alcohol. The values from hour 1 to hour 13 are calculated by taking 2 multiplied by o(n).

DT consumed by Alcohol(n)=2*o(n)

The column labeled “DT consumed by Yeast” is the amount of DT that was converted to Yeast biomass. At hour 0, the initial amount was 0.00. The conversion rate has been empirically set to be around 0.005 DT to grow yeast every hour. The initial YCC in the Yeast Tank was 40. The values from hour 1 to hour 13 are calculated by taking 0.005 multiplied by the quantity of y(n) minus 40.

DT consumed by Yeast(n)=0.005*[y(n)−40]

The column labeled “DT remaining” designates the amount of DT that remains the system. At hour 0, the initial amount was 0.00. The values from hour 1 to hour 13 are calculated using the equation DT produced by GA minus the DT consumed by Alcohol minus the DT consumed by Yeast.

DT remaining=produced by GA−DT converted to Alcohol−DT converted to Yeast

The column labeled % DT/% Yeast by weight is a key ratio that will be frequently be referred in this patent. The YCC conversion into % Yeast by weight has theoretically been determined to around 0.002. The values are calculated using the equation DT remaining(n) divided by z(n) divided by 0.002.

$$\frac{\% DT}{\% Yeast by weight} = \frac{\frac{DT remaining ( n )}{y ( n )}}{0.002}$$

FIGS. 3.2A-3.2D comprise a table of simulation data of the Fermenter Tank of the typical system. The column labeled “n” designates the time status at each hour. There are 52 hours intervals in this cycle. The column labeled “Total” designates the total amount of fresh mash added to the Fermenter Tank at each hour. At hour 0 there is 15,000 gallons of most active yeast slurry from the Yeast Tank. The working capacity of the Fermenter Tank is 500,000 gallons. At hour 13, the Fermenter Tank is full with a total of 483,000 gallons of grain slurry and yeast. From hour 14 to hour 52, the Fermenter Tank is set idle to allow the yeast to convert the sugar into alcohol. The column labeled “Sugar” designates the Fresh Mash Solution added every hour. At hour 0, no fresh mash has been added. From hour 1 to hour 13, 36,000 gallons of fresh mash solution are added every hour. From hour 14 to hour 52 no more fresh mash Solution is added. The column labeled “y(n)” designates the YCC at the beginning of the hour. The column labeled “t(n)” designates the time at which the yeast growth curve has value y(n). The column labeled “z(n)” designates the YCC at the end of the step, namely 1 hour after t(n). At hour 0, the optimal yeast budding condition was chosen. The value at hour 10 in FIGS. 3.1A and 3.1B is 152. The calculation process for columns y(n), t(n), and z(n) from hour 1 to hour 52 are three steps in this order.

First, to calculate “y(n)”, substitute all known corresponding values into the dilution formula below.

$$y ( n ) = z ( n - 1 ) * \frac{Total ( n - 1 )}{Total ( n )}$$

Second, to calculate “t(n)”, substitute all the known corresponding values into third equation. Third, to calculate “z(n)”, substitute all the known corresponding values into equation 1 with the value for t is equal t(n)+1. The column labeled “dy/dt” designates the YCC rate increase in the fermenter tank. At hour 0, the optimal yeast budding condition was chosen. The value at hour 10 in FIGS. 3.1A and 3.1B is 24.91. It will use equation 2 with t=t(n) to calculate the values from hour 1 to hour 52. The column labeled “o(n)” designates the amount of alcohol at the beginning of the hour. The column labeled “q(n)” designates the time at which the alcohol curve has the value of o(n). The column labeled “p(n)” designates the amount of alcohol at the end of the step, namely 1 hour after t(n). At hour 0, the optimal yeast budding condition was chosen. The value at hour 10 in FIGS. 3.1A and 3.1B is 0.03. The calculation process for columns “o(n)”, “q(n)”, and “p(n)” from hour 1 to hour 52 are three steps in this order.

First, to calculate “o(n)”, substitute all known corresponding values into the dilution formula below.

$$o ( n ) = p ( n - 1 ) * \frac{Total ( n - 1 )}{Total ( n )}$$

Second, to calculate “q(n)”, substitute all the known corresponding values into equation 3. Third, to calculate “p(n)”, substitute all the known corresponding values into equation 1 with the value for t equal to q(n)+1. The column labeled “do/dt” designates the alcohol rate increase in the fermenter tank. At hour 0, the optimal yeast budding condition was chosen. The value at hour 10 in FIGS. 3.1A and 3.1B is 0.01. It will use equation 2 with t=q(n) to calculate the values from hour 1 to hour 52. The column labeled “DT produced by GA” is the amount of DT that was additionally created by GA. At hour 0, the optimal yeast budding condition was chosen. The value at hour 10 in FIGS. 3.1A and 3.1B is 5.00. The conversion rate has been empirically set to be 0.5% DT every hour. The values from hour 1 to hour 52 are calculated by adding 0.5 after each hour.

DT produced by GA(n)=DT produced by GA(n−1)+0.5

The column labeled “DT converted to Alcohol” is the amount of DT that was converted to Alcohol. At hour 0, the optimal yeast budding condition was chosen. The value at hour 10 in FIGS. 3.1A and 3.1B is 0.05. The conversion rate has been empirically set to be around 2 DT for every alcohol every hour. The values from hour 1 to hour 52 are calculated by taking 2 multiplied by p(n).

DT converted to Alcohol=2*p(n)

The column labeled “DT converted to Yeast” is the amount of DT that was used by Yeast. At hour 0, the optimal yeast budding condition was chosen. The value at hour 10 in FIGS. 3.1A and 3.1B is 0.56. The conversion rate has been empirically set to be around 0.005 DT to grow yeast every hour. The initial YCC in the Yeast Tank was 40. The capacity of the Yeast Tank was 15,000 gallons. The values from hour 1 to hour 52 are calculated using the equation of 0.005 multiplied by the quantity of y(n) minus 40 multiplied by 15,000 divided by the Total(n).

$$DT converted to Yeast = 0.005 * \frac{[ y ( n ) - 40 ] * 15 \text{,} 000}{Total ( n )}$$

The column labeled “DT remaining” designates the amount of DT that is in the system. At hour 0, the optimal yeast budding condition was chosen. The value at hour 10 in FIGS. 3.1A and 3.1B is 0.56. The values from hour 1 to hour 52 are calculated using the equation DT produced by GA minus the DT consumed by Alcohol minus the DT consumed by Yeast. The best condition for alcohol production is when the DT remaining does not reach higher than 4

DT remaining=DT produced by GA−DT converted to alcohol−DT converted to Yeast

The column labeled % DT/% Yeast by weight is a key ratio that will be frequently be referred to. The conversion rate has empirically been set to be around 0.002. The values from hour 1 to hour 52 are calculated using the equation DT remaining(n) divided by z(n) divided by 0.002.

$$\frac{\% DT}{\% Yeast by weight} = \frac{\frac{DT remaining ( n )}{y ( n )}}{0.002}$$

Yeast data was procured from an ethanol producing facility. The YCC at 10 hour ranged from 90 to 120 with the average at 100. The YCC at 22 hour ranged from 200 to 250 with the average at 220. Using these two data points as references, the unique values of A, mu (μ), and lambda (λ) in the Zeiterling equation for yeast were determined to be 250, 25, and 6, respectively.

The yeast data from FIGS. 3.1A, 3.1B and 3.2A-3.2D show that the yeast growth through a five stage cycle. Stage 1 is the yeast adjustment stage where the yeast needs about two hours to adjust to a new environment before it can grow rapidly. This is shown through the minimal YCC rate change from 0 at hour 1 to 0.90 at hour 3 in FIGS. 3.1A and 3.1B. Stage 2 is the yeast growth stage where the yeast begins to multiply. This is shown through the increasing YCC rate change from 2.95 at hour 4 to 24.55 at hour 9 in FIGS. 3.1A and 3.1B. Stage 3 is the yeast most active stage where the optimal % DT/% Yeast ratio produces the highest budding conditions doubling the YCC in less than two hours. This is shown through the high YCC rate change of 24.96 at hour 10 in FIGS. 3.1A and 3.1B. Stage 4 is the yeast decline stage where the % DT, % DT/% Yeast by weight, and yeast rate change all decrease because the production of food by GA is lower than the consumption by yeast and alcohol. This is shown through the decreasing YCC rate change from 24.91 at hour 10 to 1.07 at hour 26 in FIG. 3.2A-D. Stage 5 is the yeast starving stage where the yeast dies because there is no more food. This is shown through the minimal YCC rate change from 0.82 at hour 27 to 0.00 at hour 52 in FIG. 3.2A-D.

Alcohol data is procured from an ethanol producing facility. The % Alcohol at 22 hour ranges from 4.5% to 5.5% with the average at 5.0%. The % Alcohol at 36 hour ranges from 9.0% to 11.0% with the average at 10.0%. Using these two data points as references, the unique values of A, mu (μ), and lambda (λ) in the Zeiterling equation (3) for alcohol are determined to be 12.5, 0.5, and 18, respectively.

In FIGS. 3.2A-D, % DT remaining starts to increase after hour 37. This indicates that the rate of fermentation is too fast based on the S-shape curve assumption. The yeast has “out-run” the enzyme and went into stationary phase when % DT went too low. Glucoamylase is still converting carbohydrates into glucose but it cannot catch up to the alcohol production level under the S-shape curve assumption. In a real production fermenter, the actual alcohol production would sharply drop as the S-shape curve no longer applies and the alcohol production will continue to slow down where % DT produced by GA after fermentation passes the maximum alcohol production by GA. The % DT will continue to gradually decrease and reach close to zero at the end of fermentation. In this simulation, the % DT continues to increase after hour 37 because the % DT continues to increase at 0.5% DT every hour where the simulation assumes the liquefied starch is available for the GA to covert to DT. In a real operation, the 0.5% DT increase per hour will graduate decrease to almost zero at end of fermentation, when amount of available starch graduate decrease to zero when the starch is used up at end of fermentation.

FIG. 3.3 is a plot of % DT (Y1) and % DT/% Yeast by weight (Y2) vs Time (X) of simulation data from the Fermenter Tank of the typical system. First, note that the % DT produced by GA increases constantly every hour as shown by the straight line assuming the sugar solution is not used up and is available for the enzyme. The % DT will sharply decrease when the sugar is very low then completely level off when all the sugar is used up where the slope can be varied depending on the type and dosage of the enzyme.

Second, note the % DT consumed by Alcohol is the standard S shaped curve. It reaches a level plateau around hour 45 where the % DT change is less than 1% [(25.48-25.02)/25.02] which where the yeast has reached the starving phase.

Third, note that the % DT consumed by Yeast production is the standard S shaped curve. It reaches a level plateau around hour 23 where the % DT change is less than 1% [(1.21−1.20)/1.21] which is where the yeast switches from a growing more yeast to producing alcohol.

Fourth, note that the % DT remaining reaches above 8% at hour 10 and continues to stay that high to hour 16. Currently dry mill plants reach a peak % DT around hour 13 to hour 20 with a high point of 8% to 12%. In the typical System, the % DT value is too high as the yeast becomes stressed with too much sugar to convert. This stress forces the yeast to produce excess glycerol which measurably reduces the alcohol yield.

Fifth, note that the % DT/% Yeast by weight starts with a value of 14.39 from the Yeast Tank, reaches a high peak value 66.93 at hour 3, and gradually decreases to a low point close to 0.00 at the end of fermentation. This curve is consistent with most dry mill plants.

FIG. 3.4 is a plot of YCC (Y1) and % Alcohol (Y2) and % DT/% Yeast by weight (Y2) vs Time (X) of simulation data from the Fermenter Tank of the typical system. First, note that the YCC is 152 from Yeast Tank at hour 0 then dropped to 42 for the first two hours. The Fresh Mash Solution dilutes the Yeast Solution and yeast endures shock requiring two hours to adjust in the new environment before more yeast can grow.

Second, note that the YCC increases in an upward trend when the % DT/% Yeast by weight is between 10 and 50. Third, note that the % Alcohol production increases in an upward trend when the % DT/% Yeast by weight is between 0 and 10.

FIG. 3.5 is a plot of YCC rate change (Y1) and % DT/% Yeast by weight (Y1) and Alcohol rate change (Y2) vs Time (X) from the Fermenter Tank of the typical system simulation data. First, note that the YCC rate change reaches a maximum of 24.96 at hour 8 with a % DT/% Yeast by weight of 34.45. Second, note that the % Alcohol rate change reaches a maximum of 0.50 at hour 23 and hour 24 with a % DT/% Yeast by weight of 8.95 and 7.78, respectively.

The initial YCC at hour 0 for the typical system in the 15,000 gallon Yeast Tank was 40. The final YCC at hour 10 in the 15,000 gallon Yeast Tank was 152. The Yeast Solution is then dumped into the Fermenter Tank. Therefore the yeast growth in the Yeast Tank is 3.80 (152/40) times. The expected final YCC at the end of 52 hours in the 483,000 gallon fermenter tank was 250. Therefore the yeast growth in the Fermenter Tank is 52.96 times [(250*483,000)/(152*15,000)]. Therefore, the yeast has to grow 13.94 (52.96/3.80) times more in the Fermenter Tank than in the Yeast Tank. This is a lot of the yeast growth required in the Fermenter Tank which ends up delaying alcohol production.

It was noted in a paper that higher glucose (DT above 4%) will create osmotic stress on yeast which will hinder the alcohol production in two ways: (1) increase the byproduct Glycerine production rate and (2) decrease in the yeast growth rate.

FIG. 4 is a plot of data of the byproduct Glycerine for the typical system. First, note that amount of Glycerine production rate is higher during the first 25 hours and substantially slower later in the fermentation event. Typically, a higher % DT concentration (8% to 12%) during first 25 hour creates yeast stress and produces more Glycerine (1.2% to 1.5%). It is showed that the split or continuous enzyme dosage methods during the fermenter tank filling cycle is able to control the % DT in the early fermentation stage. As a result, the Glycerine production decreased from 1.3% to 0.8%. The Poet has shown that using raw starch to slow down the % DT production can cut the Glycerine production. However, a negative of that teaching is that a much longer fermentation time is needed. In addition, the raw starch process has a higher possibility of losing excess starch into DDGS.

One way to counteract the problem of high % DT in Yeast Tank and Fermenter Tank is to increase the yeast concentration in Yeast Tank or speed up the yeast growth rate in fermenter tank. This can be done by few ways: (1) initially add more dry yeast in the Yeast Tank, (2) dump yeast solution from the Yeast Tank into the Fermenter Tank multiple times, and (3) dump a majority of the yeast solution (70% to 80%) from the Yeast Tank into the Fermenter Tank then keep the remaining solution in the Yeast Tank to grow more Yeast Solution, and (4) hold the propagation tank longer before transfer to get the highest cell count before transfer.

Initially, within the biological limits of the system, adding double the amount of the dry yeast will double the amount of yeast growth in the Yeast Tank, which will double the amount of yeast dumped from the Yeast Tank to the Fermenter Tank, and result in doubling the alcohol production rate in the Fermenter Tank. When the YCC is doubled, however, the % DT/% Yeast by weight value will in turn be halved. The yeast growth rate will in turn decrease and the yeast will not grow to the highest budding concentration. This has resulted in slight improvements to lowering the % DT in the Fermenter Tank and a slight increase in alcohol yield, yet the required two fold increase of raw materials (dry yeast and enzyme) compared to the typical system do not justify the minimal commercial gain.

Dumping the yeast from the Yeast Tank into the Fermenter Tank more than once in a 13 hour filling period may increase the initial YCC in the Fermenter Tank, but the yeast is likely weak as it did not reach its optimal budding condition. Again, this has resulted in slight improvements to lowering the % DT in the Fermenter Tank and slight increases in alcohol yield, yet the required multi-fold increase of raw materials (dry yeast and enzyme) compared to the typical system is not justified especially with the increase of cost of dry yeast and enzyme.

FIGS. 5.1A and 5.1B comprise a table of simulation data of the Yeast Tank single batch of the 80% Refill and Dump System. The difference between FIG. 3.1A-3.1B and FIGS. 5.1A-5.1B is at hour 0, 200 pounds of dry yeast is added. The conversion factor is still 2.5 pounds per dry yeast per 15,000 gallons. Therefore the Yeast Tank has an YCC of 80 (200 divided by 2.5). As a result, the new A, mu, and lambda for yeast were determined to be 500, 50, and 6, respectively. The values of A, mu, and lambda for alcohol remained the same 12.5, 0.5, and 18, respectively.

FIGS. 5.2A and 5.2B comprise a table of simulation data of the Yeast Tank continuous feed of the 80% Refill and Dump System. The calculations for each column are similar to FIGS. 3.2A-3.2D.

FIGS. 5.3A-5.3D is a table of simulation data of the Fermenter Tank of the 80% Refill and Dump System. The calculations for each column are similar to FIGS. 3.2A-3.2D.

FIG. 5.4 is a plot of DT (Y1) and % DT/% Yeast by weight (Y2) vs Time (X) from the Fermenter Tank of the Yeast Tank 80% Dump and Refill System. Note that the % DT values are all over 6% from hour 2 to hour 18 with the highest 8.07 at the end of the filling period. The yeast is stressed and unable to convert the large amount of sugar into alcohol despite the large initial yeast quantity. This indicates that the yeast from the 80% Yeast Dump and Refill System that got dumped was not active, strong, and healthy.

FIG. 5.5 is a plot of YCC (Y1) and % Alcohol (Y2) and % DT/% Yeast by weight (Y2) vs Time (X) from the Fermenter Tank of the Yeast Tank 80% Dump and Refill System. First, note that the YCC from hour 0 to hour 1 in the Fermenter Tank experiences a severe drop from 431 to 155. The yeast further drops to its lowest point of 134 in hour 2. This suggests that the yeast continues to experience great shock after the dump.

FIG. 5.6 is a plot of YCC rate change (Y1) and % DT/% Yeast by weight (Y1) and % Alcohol rate change (Y2) vs Time (X) from the Fermenter Tank of the Yeast Tank 80% Dump and Refill System. First, note that the YCC rate change is the highest at hour 8 with a value of 49.93. This indicates that keeping the best YCC rate change occurs in the filling period between hour 5 and hour 15. Second, note that the % Alcohol rate change is the highest at hour 21 and hour 22 with a value of 0.50. This is at the very end of the ideal alcohol production period between hour 15 and 25. This signifies that alcohol production is late and will not complete to maximize the yield.

FIGS. 5.4 to 5.6 show two fundamental problems with the Yeast Tank 80% Dump and Refill System. First, note that the yeast in the Fermenter Tank has % DT that reaches 8.07%, which is too high a concentration for good yeast activity. There is too much yeast shock in the system, which stresses the yeast resulting in production of excess Glycerine. Second, note that the Yeast Tank in the refilling stage has % DT/% Yeast by weight at dangerously low values (1.06 to 6.72). This yeast begins to make alcohol instead of the regenerating more yeast. As a result, the new yeast is not as healthy, strong or active. The one positive aspect of this method is that less raw materials of dry yeast will be used leading to lower costs.

Typical wet mill plants have used a centrifuge to recycle the yeast to maintain a high YCC and maintain right % DT/% Yeast by weight. In order to apply the same technology in dry grind plants, the solid (fiber, germ, and protein) must first be removed. This process of very complicated and costly. Alternatively, wet mills use continuous fermentation to get high cell counts at all times. The negative of this is continuous fermentation systems do not have the ability to clean out the fermentation system and build high bacterial concentrations within the fermentation system over time resulting in yield loss due to continual bacterial growth.

FIG. 6.1 is a table that shows the maximum level of DT that can be in a system given a desired Yeast amount and % DT/% Yeast by weight. The highest budding condition for yeast growth occurs when the % DT/% Yeast by weight is 10. The % DT/% Yeast by weight can widely range depending on type of yeast and operation conditions such as pH, temperature, type of yeast, and nutrition. The % DT can be at 2%, 3%, 4%, and 5% in order to be able to produce YCC of 100, 150, 200, and 250, respectively. It has been scientifically shown the best yeast growing conditions occur when the system maintains a % DT of less than 2%. When the % DT above 2%, the yeast is stressed and the growth becomes suppressed. The ultimate goal is to have the healthiest and highest practical concentration of yeast in the Fermenter Tank. This creates a tradeoff to produce either very strong YCC of 100 to 150 each cycle or less strong YCC of 200 to 250 each cycle. As a result, keeping % DT as the new set parameter will reduce the shock and explain how to best grow the yeast. Following the % DT/% Yeast by weight column shows that in order to maintain YCC of 200, 300, and 400, the % DT must be kept at 2%, 3% and 4%, respectively. This means keeping % DT below 2% will produce a maximum YCC to 200 and produce a minimal amount of by product (Gylcerine). In typical operation, yeast is grown in Yeast Tank around 6 hour to 10 hour and the yeast slurry is dumped into fermenter at a YCC around 100 to 150 of the most active (highest budding) yeast where the % DT is 2% to 3%. Another option is to grow a high YCC (150 or higher) that is less active (lower budding) where the % DT is 3% to 6% and the % DT/% Yeast by weight is 5 to 10. When the yeast slurry is dumped into the Fermenter Tank, the yeast needs at least two hour to adjust the new environment. Meanwhile, the % DT continues to increase during the whole 13 hour filling cycle and in most cases beyond that (up to 26 hour). The % DT can reach 8% to 12% which creates yeast stress and slows down the yeast growing rate and alcohol production rate.

FIG. 6.2 is a plot of DT (Y) vs YCC (X). The linear relationships show the dilemma between yeast and sugar concentrations. It shows it is not possible to have the most active yeast and a higher YCC sent to the Fermenter Tank at the same time. In the typical System, a choice must be made of either taking the most active yeast at a lower YCC or less active yeast at a higher YCC.

### SUMMARY OF THE INVENTION

The present invention relates to methods of growing higher and stronger levels of yeast in the Yeast Tank and Fermenter Tank during the fermentation filling cycle.

In some embodiments, yeast growth is reconfigured by continuing pumping yeast under the most active conditions while at a lower YCC (yeast cell count) into the Fermenter Tank during a filling period. This keeps both the Yeast Tank and the Fermenter Tank in their most active conditions while increasing the YCC simultaneously. In some embodiments, at least 2 times more of yeast is pumped from the Yeast Tank to the Fermenter Tank.

A computer simulation is created using plant capacity/rate specifications, raw input/output data, and mathematical modeling. This data replication tracked the process and provided insight on calculations. In addition, a measurable and useful parameter, % DT/% Yeast by weight ratio (or “food” to yeast ratio), is also introduced. (e.g., % DT=glucose) This ratio offers information on the health status of the yeast after every hour and a method of smoothly transferring the yeast from yeast growing phase to alcohol producing phase during a fermenter filling period, such that shocks to the yeast is able to be avoided.

The ratio starts with a high value (more than 50, where the amount of sugar is 50 times more than yeast) and gradually decreases close to zero (little sugar and large yeast, where the yeast starves and dies) at end of the fermentation. During the yeast growth phase, a proper adjustment for % DT/% Yeast by weight is needed to produce most active health yeast at highest rate. If the % DT/% Yeast by weight is too high (too much % DT), the yeast can be stressed, which can result in slowing the yeast growth rate and a produce an unwanted by-product (Glycerin instead of Alcohol). If the % DT/% Yeast is too low, the yeast will starve, which can result in slowing the yeast growth rate and alcohol production rate, and eventually the yeast dies.

Knowing the ratio value of the food concentration divided by the yeast by weight after every hour of production is a powerful tool giving plant engineers the necessary information to then adjust various mechanisms to better stabilize the ratio in the system at selected time intervals. These mechanisms include varying the sugar and yeast concentrations, sugar flow rates, enzyme dosage, pH, and temperature. Three processes are disclosed herein, which can be used to maximize alcohol production. A computer simulation provides tracking of the % DT/% Yeast by weight, which provides promising data verifying more alcohol yield.

Zweiterling et al (1990) empirically originated an S curve (equation 1) that best describes yeast growth or alcohol production. This formula introduced three constant parameters. A is the theoretical maximum amount of alcohol that the system produces. Mu (g) is the slope of the curve or linear max ethanol rate. Lambda (A) is the x-axis intercept time where the line drawn to indicate the mu slope crosses.

$$y = A {e}^{- {e}^{[ \frac{μ e}{A} ( λ - t ) + 1 ]}} \qquad ( equation 1 )$$

The derivative of Zweiterling et al (1990) S curve (equation 2) is the Yeast Cell Count (YCC) rate change or alcohol rate change.

$$\frac{d y}{d t} = {e}^{- {e}^{[ \frac{μ e}{A} ( λ - t ) + 1 ]}} * {e}^{[ \frac{μ e}{A} ( λ - t ) + 1 ]} * μ e \qquad ( equation 2 )$$

For given yeast growth or alcohol production, y, the time t at which the Zweiterling et al (1990) S curve equation (equation 1) yield value y can be analytically solved.

$$t = λ - \frac{A}{μ e} ⌈ ln ( ln ( \frac{A}{y} ) ) - 1 ⌉ \qquad ( equation 3 )$$

In a first aspect, the significance of the % DT/% Yeast by weight ratio is used as a controlling metric. The % DT/% Yeast by weight is used to reevaluate the problems with the inefficiencies of the typical systems.

In a second aspect, the budding condition is improved in the Yeast Tank after hour 7 to hour 13 using a continuous flow of Yeast Solution from the Yeast Tank. The process includes sending part of the Fresh Mash feed into the Yeast Tank and discharge the exact same volume of Fresh Mash into the Fermenter Tank in/during the filling period. The process continues to add the remaining part of the Sugar Solution into the Fermenter Tank in/during the filling period. The tanks are rotated when the Fermenter Tank is full and this process is repeated. A main advantage is that a steady stream of yeast solution fed into the Fermenter Tank maintains the yeast to be the most alive and active compared to the one-time dump process. By providing a steady steam, the yeast is able to experience much less shock.

In a third aspect, the budding condition is improved in the yeast tank after hour 7 to hour 13 using continuous flow of the fermenter solution from the aged fermenter tank. The process includes sending part of the sugar solution feed into the aged fermenter Tank and discharge the exact same volume of fermenter solution into a new/second fermenter tank for the filling period. The process would continue to add the remaining part of the sugar solution into the new/second fermenter tank for/during the filling period. The tanks are rotated when the fermenter tank is full and this process is repeated. The main advantage is that a steady stream of fermenter solution fed into the new fermenter tank maintains the yeast to be the most alive and active compared to the one-time dump process. By providing a steady steam, the yeast is able to experience much less shock.

In a fourth aspect, the budding condition is improved in the fermenter tank after hour 7 to hour 13 by using both continuous flows from the yeast tank and aged fermenter Tank. This is a combination of the yeast tank improvement system and fermenter tank improvement system. In some embodiments, the process includes (1) sending part of the Fresh Mash feed into the Yeast Tank and discharge approximately the same volume of yeast solution from the yeast tank into the fermenter tank during/for the filling period and (2) sending part of the Fresh Mash feed into the aged fermenter tank and discharge approximately the same volume of aged fermention broth into the new/second fermenter tank during the filling period. The process can continue to add the remaining part of the Fresh Mash into the new/second fermenter tank during/for the filling period. The tanks are rotated when the fermenter tank is full and the process is repeated. The main advantage is that steady streams of both the yeast solution and fermenter solution maintain the yeast to be the most alive and active compared to the one-time dump process. By providing a steady steam, the yeast is able to experience much less shock.

In a fifth aspect, a higher concentration of yeast slurry is added from the aged fermenter tank to fill new/second fermenter tank via the CO₂ froth layer at the top of the aged fermenter. The CO₂ frothy layer contains the highest active yeast at the top of the aged fermenter tank. When the aged fermenter tank is filled to a full capacity, the frothy layer (liquid with most active yeast) is sent as an overflow stream allowing it to supply the new/second fermenter tank with active yeast while not adding large amounts of fermentation broth from the aged fermenter tank.

In a sixth aspect, higher concentration of active and acclimated yeast is added by transferring yeast from an aged fermenter to a younger, filling fermenter, which has advantageous properties including: (1) higher cell concentrations, (2) yeast acclimated to the fermentation broth conditions, (3) yeast cell walls and cell membranes acclimated to higher ethanol concentrations, (4) highly active yeast that are in the log growth state, (5) glucoamylase enriched media, (6) DT immediately available, (7) lower viscosity enhancing mass transfer, and (8) higher potential for complex nutrient release due to presence of growing population of old yeast starting to undergo cell lysis.

During the initial filling cycle of a new fermenter, a portion of fermenting or fermented broth from one or more aged fermenter, including potentially the beerwell, is transferred from the aged fermenter(s) and/or beerwell into the filling fermenter. The amount of broth transferred from the aged fermenter(s) and/or beerwell can be from 0 to 99% the volume of the newly filling fermenter. In some embodiments, the amount of broth transferred from the aged fermenter(s) and/or beerwell is from 51% to 99% the volume of the newly filling fermenter. In other embodiments, the amount of broth transferred from the aged fermenter(s) and/or beerwell is from 2% to 50% the volume of the newly filling fermenter. In some other embodiments, the amount of broth transferred from the aged fermenter(s) and/or beerwell is between 2% and 30% the volume of the newly filling fermenter.

In some embodiments, a liquid is used to replace some or all of the fermentation broth transferred from any aged fermenter tank or beerwell. The liquid is able to be fresh mash, newly added carbohydrate and nutrients introduced into the tank, a fermentation broth from any of the fermenter tank(s) and/or beerwell, and a low-fermentable liquid. A person of ordinary skill in the art would understand that any proportion of liquid from any of these sources can be added back into tanks in any proportion predetermined. This method described above provides advantages in continuous fermentation systems, which provides, at the beginning stage of fermentation, a high concentration highly active yeast, such that a robust start to the fermentation is induced. The advantages include, for example: (1) reduced risk of bacterial growth, (2) reduced need for glucoamylase, and (3) a shorter total fermentation time. The method overcomes the negative aspect of continuous fermentation by introducing control points including (1) the amount of the fermenter broth to be added, (2) the supplying source of the recycled fermenter broth, (3) the timing of bringing the recycled fermenter broth into the newly filling fermenter, and (4) a method of breaking the bacterial growth cycle by interrupting recycle of yeast when bacterial concentrations rise higher than desired. These control points allow the fermentation system to have the advantages of a true batch-process including (1) low bacterial contamination and (2) higher final ethanol concentrations along with the recognized advantages of a continuous fermentation process. The advantages include (1) rapid start to fermentation of fresh mash and (2) faster overall fermentation time.

In some embodiments, the present invention includes three systems focusing on developing new configurations that produce five key yeast improvements. First, the yeast is able to grow as fast as possible in the yeast tank prior to transferring into the fermenter tank. Second, the yeast tank continues to pump the most active yeast to the fermenter tank at the highest rate while maintaining optimal yeast tank conditions. Third, the yeast experiences minimal shock by adjusting the sugar rate and enzyme dosage in the fermenter tank during the 13 hour filling cycle especially at the beginning of first 6 hours. Fourth, the yeast undergoes a smooth transition between highest yeast growth rate to a gradual decreased yeast growth rate to an increased alcohol production rate during the first 13 hour filling period. Fifth, the enzyme dosage and fresh mash rate sent to the yeast tank and/or fermenter tank during the first 13 hour cycle ensure both the yeast tank and fermenter tank are in the most active conditions. Sixth, undesired bacterial growth over time can be controlled by changing the location(s) and volume(s) from where the recycle yeast stream comes from with the ability to have zero recycle stream for a fermenter batch to completely break the cycle as needed. In some embodiments, the factor of % DT/% Yeast by weight is used as a guideline to maintain and control the systems.

In an aspect, a fermentation method comprises adjusting the health condition of yeast in a yeast solution based on a ratio of % DT/% Yeast by weight and continuously inputting the yeast solution in a fermenter tank during a filling period. In some embodiments, the ratio of the % DT/% Yeast by weight is adjusted to optimize the health condition of the yeast condition. In other embodiments, the health condition comprises an active condition of the yeast in the yeast solution. In some other embodiments, the method further comprises adjusting the ratio of % DT/% Yeast by weight, such that the fermenter tank generates less glycerin. In some embodiments, the method further comprises preventing the ratio of the % DT/% Yeast by weight exceed a higher threshold to prevent a stress of the yeast. In some other embodiments, the method further comprises preventing the ratio of the % DT/% Yeast by weight below a lower threshold to prevent a death of the yeast. In some embodiments, the ratio of the % DT/% Yeast by weight is adjusted based on a sugar and the yeast concentration. In other embodiments, the ratio of the % DT/% Yeast by weight is adjusted based on a sugar flow rate, an enzyme dosage, a pH value, and a temperature of the fermenter tank.

In another aspect, a fermentation method comprises providing a continuous flow of a yeast solution in a fermenter tank during a filling period monitoring a ratio of % DT/% Yeast by weight, and adjusting a rate of the continuous flow based on the ratio. In some embodiments, the continuous flow of a yeast solution is from a yeast tank. In other embodiments, the method further comprises sending a first volume of a fresh mash feed to the yeast tank. In some other embodiments, the method further comprises sending a second volume of the yeast solution from the yeast tank to the fermenter tank, wherein the second volume is the same as the first volume.

In another aspect, a method of fermentation tank improvement comprises providing a first amount of a sugar solution to a first fermenter tank and a second amount of the sugar solution to a second fermenter tank, providing a third amount of a fermenting solution from the second fermenter tank to the first fermenter tank, wherein the third amount is equal to the first amount. In some embodiments, the method further comprises sending a fourth amount of yeast solution from a yeast tank to the first fermenter tank. In other embodiments, the first fermenter tank is a new fermenter tank and the second fermenter tank is an aged fermenter tank at a first time period. In some embodiments, the first fermenter tank receives the firth amount of a sugar solution. In other embodiments, the third fermenter tank is a new fermenter tank and receives the yeast solution from the yeast tank.

In another aspect, a method of fermentation tank and yeast tank improvement comprises providing a first amount of a fresh mash feed to a yeast tank, providing a second amount of the fresh mash feed to an aged fermenter tank, providing the first amount of a yeast solution from the yeast tank to a first young fermenter tank, and providing the second amount of a first fermenting solution from the aged fermenter tank to the first young fermenter tank. In some embodiments, the first young fermenter tank is used as a second aged fermenter tank at a next time period. In other embodiments, the second aged fermenter tank is used to provide a second fermenting solution to a second young fermenter tank.

In another aspect, a method of enhancing fermentation process comprises selectively taking a concentrated yeast slurry from the CO₂ froth layer at a top portion of an aged fermenter, and adding the yeast slurry to a new fermenter. In some embodiments, the CO₂ froth layer comprises a portion of higher active yeast than the remaining yeasts in a yeast tank. In other embodiments, the concentrated yeast slurry is in an overflow stream from the yeast tank.

In another aspect, a fermentation system comprises multiple fermentation tanks including an aged tank and a young tank and a yeast tank, wherein the yeast tank provides a yeast solution and the aged tank provide a fermenting solution to the young tank. In other embodiments, the system further comprises a sugar solution providing source providing a sugar solution to both the young tank and the aged tank.

### BRIEF DESCRIPTION OF THE DRAWINGS

Embodiments will now be described by way of examples, with reference to the accompanying drawings which are meant to be exemplary and not limiting. For all figures mentioned herein, like numbered elements refer to like elements throughout.

FIG. 1 is a flow diagram of a typical dry grind ethanol process.

FIGS. 2.1 to 2.4 comprise flow diagrams of the typical system from 0 hour to 52 hour.

FIGS. 3.1A and 3.1B comprise a table of the Yeast Tank of the typical system simulation data.

FIGS. 3.2A-3.2D comprise a table of the Fermenter Tank of the typical system simulation data.

FIG. 3.3 is a plot of DT (Y1) and % DT/% Yeast by weight (Y2) vs Time (X) from the Fermenter Tank of the typical system simulation data.

FIG. 3.4 is a plot of YCC (Y1) and % DT/% Yeast by weight (Y1) and % Alcohol (Y2) vs Time (X) of the Fermenter Tank of the typical system simulation data.

FIG. 3.5 is a plot of YCC rate change (Y1) and % DT/% Yeast by weight (Y1) and % Alcohol rate change (Y2) vs Time (X) of the Fermenter Tank of the typical system simulation data.

FIG. 4 is a plot of Glycerine by weight in Fermenter Tank.

FIGS. 5.1A and 5.1B comprise a table of the Yeast Tank of the 80% Dump and Refill System Before simulation data.

FIGS. 5.2A and 5.2B comprise a table of the Yeast Tank of the 80% Dump and Refill System After simulation data.

FIGS. 5.3A-5.3D comprise a table of the Fermenter Tank of the 80% Dump and Refill System simulation data.

FIG. 5.4 is a plot of DT (Y) and % DT/% Yeast by weight (Y) vs Time (X) of the Fermenter Tank of the 80% Dump and Refill System simulation data.

FIG. 5.5 is a plot of YCC (Y1) and % Alcohol (Y2) and % DT/% Yeast by weight (Y2) vs Time (X) of the Fermenter Tank of the 80% Dump and Refill System simulation data.

FIG. 5.6 is a plot of YCC rate change (Y1) and % DT/% Yeast by weight (Y1) and % Alcohol rate change (Y2) vs Time (X) from the Fermenter Tank of the 80% Dump and Refill System simulation data.

FIG. 6.1 is a table that shows the maximum level of DT that should be in a system given a desired Yeast amount and % DT/% Yeast by weight.

FIG. 6.2 is a plot of DT (Y) vs YCC (X).

FIGS. 7.1 to 7.4 comprise flow diagrams of Low Yeast Tank Improvement for a 52 hour cycle in accordance with some embodiments of the present invention.

FIGS. 8.1A and 8.1B comprise a table of the Yeast Tank single batch of the Low Yeast Tank Improvement System simulation data in accordance with some embodiments of the present invention.

FIGS. 8.2A and 8.2B comprise a table of the Yeast Tank continuous feed of the Low Yeast Tank Improvement System simulation data in accordance with some embodiments of the present invention.

FIGS. 8.3A-8.3D comprise a table of the Fermenter Tank of the Low Yeast Tank Improvement System simulation data of a 500,000 gallon fermenter in accordance with some embodiments of the present invention.

FIG. 8.4 is a plot of DT (Y1) and % DT/% Yeast by weight (Y2) vs Time (X) of the Fermenter Tank of Low Yeast Tank Improvement System simulation data in accordance with some embodiments of the present invention.

FIG. 8.5 is a plot of YCC (Y1) and % Alcohol (Y2) and % DT/% Yeast by weight (Y2) vs Time (X) of the Fermenter Tank of Low Yeast Tank Improvement System simulation data in accordance with some embodiments of the present invention.

FIG. 8.6 is a plot of YCC rate change (Y1) and % DT/% Yeast by weight (Y1) and Alcohol rate change (Y2) vs Time (X) of the Fermenter Tank of Low Yeast Tank Improvement System simulation data in accordance with some embodiments of the present invention.

FIGS. 9.1A and 9.1B comprise a table of the Yeast Tank single batch of the High Yeast Tank Improvement System simulation data in accordance with some embodiments of the present invention.

FIGS. 9.2A and 9.2B comprise a table of the Yeast Tank continuous feed of the High Yeast Tank Improvement System simulation data in accordance with some embodiments of the present invention.

FIGS. 9.3A-9.3D comprise a table of the Fermenter Tank of the High Yeast Tank Improvement System simulation data of a 500,000 gallon fermenter in accordance with some embodiments of the present invention.

FIG. 9.4 is a plot of DT (Y1) and % DT/% Yeast by weight (Y2) vs Time (X) of the Fermenter Tank of the High Yeast Tank Improvement System simulation data in accordance with some embodiments of the present invention.

FIG. 9.5 is a plot of YCC (Y1) and % Alcohol (Y2) and % DT/% Yeast by weight (Y2) vs Time (X) of the Fermenter Tank of the High Yeast Tank Improvement System simulation data in accordance with some embodiments of the present invention.

FIG. 9.6 is a plot of YCC rate change (Y1) and % DT/% Yeast by weight (Y1) and Alcohol rate change (Y2) vs Time (X) of the Fermenter Tank of the High Yeast Tank Improvement System simulation data in accordance with some embodiments of the present invention.

FIGS. 10.1 to 10.4 are flow diagrams of Fermenter Tank Improvement for a 52 hour cycle in accordance with some embodiments of the present invention. In some embodiments, the fermenter receiving material from the yeast tank is the filling fermenter.

FIG. 11.1 is a table of the Fermenter Tank Improvement System, Yeast Tank single batch simulation data in accordance with some embodiments of the present invention.

FIGS. 11.2A-11.2D comprise a table of the Fermenter Tank Improvement System, Fermenter Tank simulation data of a 500,000 gallon fermenter in accordance with some embodiments of the present invention.

FIG. 11.3 is a plot of DT (Y1) and % DT/% Yeast by weight (Y2) vs Time (X) of the Fermenter Tank of the Fermenter Tank Improvement System simulation data in accordance with some embodiments of the present invention.

FIG. 11.4 is a plot of YCC (Y1) and % Alcohol (Y2) and % DT/% Yeast by weight (Y2) vs Time (X) of the Fermenter Tank of the Fermenter Tank Improvement System simulation data in accordance with some embodiments of the present invention.

FIG. 11.5 is a plot of YCC rate change (Y1) and % DT/% Yeast by weight (Y1) and Alcohol rate change (Y2) vs Time (X) of the Fermenter Tank of the Fermenter Tank Improvement System simulation data in accordance with some embodiments of the present invention.

FIGS. 12.1 to 12.4 are flow diagrams of a Combination Improvement Systems for a 52 hour cycle in accordance with some embodiments of the present invention. In some embodiments, the fermenter receiving material from the yeast tank is the filling fermenter.

FIGS. 13.1 to 13.4 are flow diagrams of an Overflow Improvement Systems for a 52 hour cycle in accordance with some embodiments of the present invention.

FIGS. 14.1A-14.1D comprise a table of all the System simulation data in accordance with some embodiments of the present invention.

FIG. 14.2 is a plot of YCC (Y) vs Time (X) of the Fermenter Tank of all Systems simulation data in accordance with some embodiments of the present invention.

FIG. 14.3 is a plot of % Alcohol (Y) vs Time (X) of the Fermenter Tank of all Systems simulation data in accordance with some embodiments of the present invention.

FIG. 14.4 is a plot of DT (Y) vs Time (X) of the Fermenter Tank of all Systems simulation data in accordance with some embodiments of the present invention.

FIG. 14.5 is a plot of YCC rate change (Y) vs Time (X) of the Fermenter Tank for all Systems simulation data in accordance with some embodiments of the present invention.

FIG. 14.6 is a plot of % Alcohol rate change (Y) vs Time (X) of the Fermenter Tank of all Systems simulation data in accordance with some embodiments of the present invention.

FIG. 14.7 is a plot of Alcohol rate change (Y) vs Time (X) of the Fermenter Tank for all the Systems in accordance with some embodiments of the present invention.

FIGS. 15A-15F show, of the five different recycle methods modeled, the 21% recycle produces the most active yeast set up of a 650,000 gallons fermenter in accordance with some embodiments of the present invention.

FIGS. 16A-16D summarize the computer simulation output on the above five different recycle setups in accordance with some embodiments of the present invention.

FIGS. 17.1-17.5 comprise the comparison data and plots in accordance with some embodiments of the present invention.

FIGS. 18A-18D show the various % recycle on constant flow split set up are modeled on the simulation program in accordance with some embodiments of the present invention.

FIGS. 19.1-19.5 comprise a plot on various % recycle yeast slurry with constant flow split to set fermenter and fill fermenter in accordance with some embodiments of the present invention.

FIGS. 20.1-20.3 comprise a graph showing the GA savings and alcohol yield increase by constant flow split set up in accordance with some embodiments of the present invention.

FIGS. 21.1A, 21.1B, 21.2A and 21.2B comprise tables of summary data with 21% recycle yeast recycle in accordance with some embodiment of the present invention.

FIGS. 22.1-22.10 comprise figures with summary data with 21% recycle yeast on 55 MGY plant in accordance some embodiments of the present invention.

FIG. 23 is a chart showing % alcohol yield increase in accordance with some embodiments of the present invention.

FIG. 24 is a flow chart showing recycle yeast set up with low fermentable sugar, back set stream, or cook water in accordance with some embodiments of the present invention.

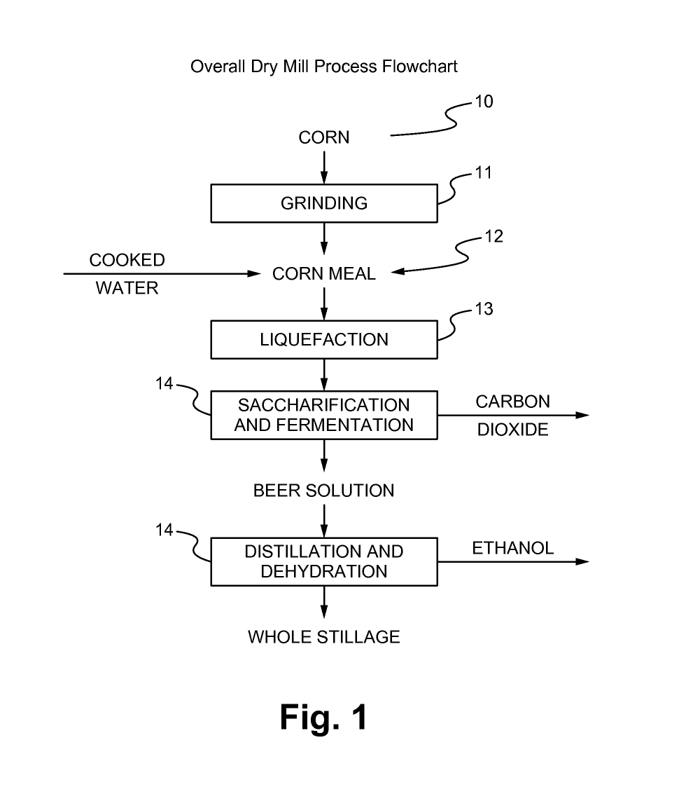

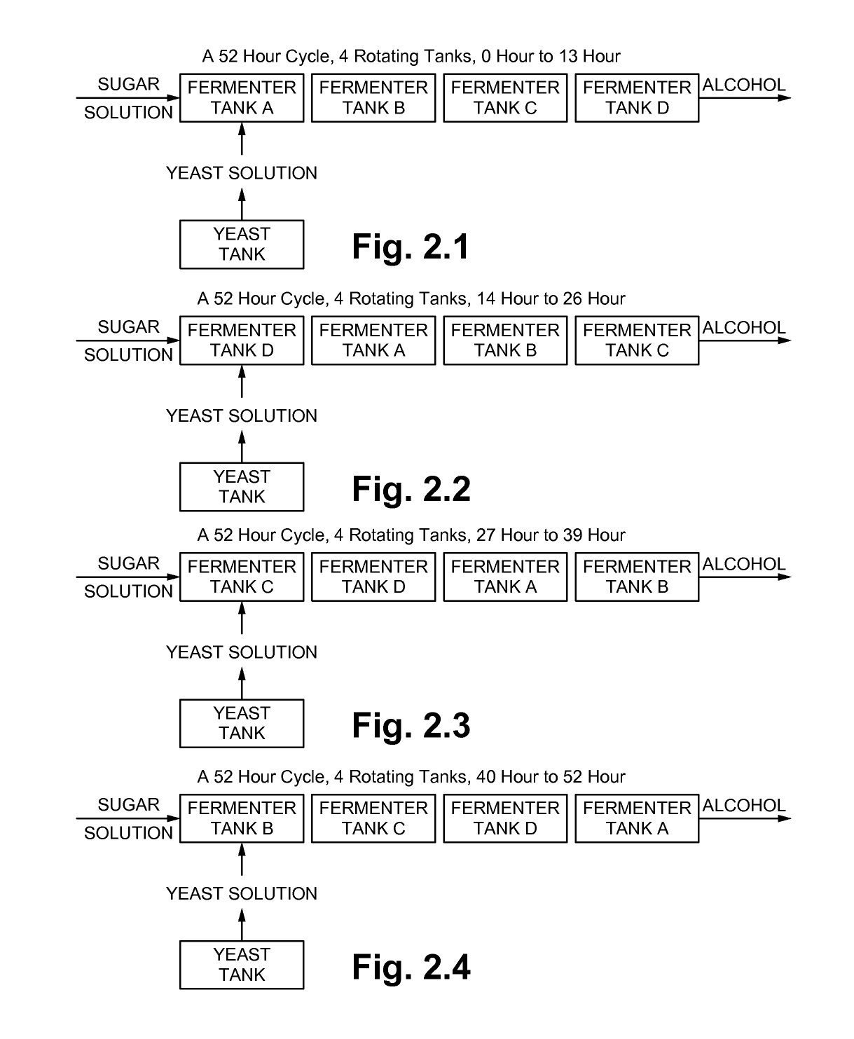

### DETAILED DESCRIPTION OF THE PREFERRED EMBODIMENT

Reference is made in detail to the embodiments of the present invention, examples of which are illustrated in the accompanying drawings. While the invention is described in conjunction with the embodiments below, it is understood that they are not intended to limit the invention to these embodiments and examples. On the contrary, the invention is intended to cover alternatives, modifications and equivalents, which can be included within the spirit and scope of the invention as defined by the appended claims. Furthermore, in the following detailed description of the present invention, numerous specific details are set forth in order to more fully illustrate the present invention. However, it is apparent to one of ordinary skill in the prior art having the benefit of this disclosure that the present invention can be practiced without these specific details. In other instances, well-known methods and procedures, components and processes have not been described in detail so as not to unnecessarily obscure aspects of the present invention. It is, of course, appreciated that in the development of any such actual implementation, numerous implementation-specific decisions must be made in order to achieve the developer's specific goals, such as compliance with application and business related constraints, and that these specific goals vary from one implementation to another and from one developer to another. Moreover, it is appreciated that such a development effort can be complex and time-consuming, but is nevertheless a routine undertaking of engineering for those of ordinary skill in the art having the benefit of this disclosure.

The computer simulation along with the proprietary raw data show that yeast grow in a more sustainable manner and convert more C₆ sugars yielding more ethanol using the methods and devices disclosed herein.

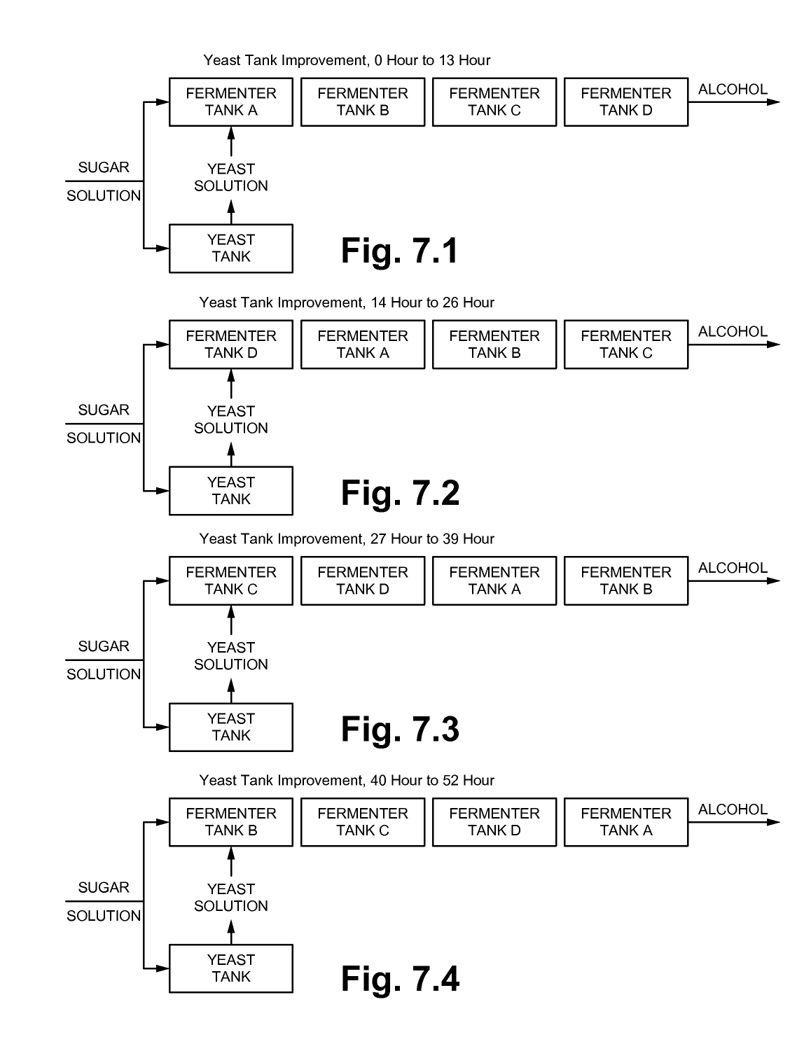

FIGS. 7.1 to 7.4 show the fermentation process flow of a Yeast Tank Improvement System of a 52 hour cycle with 4 rotating Fermenter Tanks in accordance with some embodiments of the present invention. The yeast tank fermenting system provides advantageous aspects. First, the Yeast Tank Improvement System (e.g., a yeast tank fermentation/fermenting system) has the fresh mash diverted into two streams in every cycle. The majority of the fresh mash is sent to the fermenting tank (e.g., fermenter tank and/or fermenter) while the remaining sugar solution is sent to the yeast tank with an equal amount of yeast solution sent to the fermenting tank. Second, the yeast tank fermenting system has a continuous flow of yeast solution pumping from the yeast tank to the fermenting tank every hour during hour 1 to hour 13 rather than a one-time dump. These changes enables the yeast to grow stronger in the yeast tank and maintain the high active YCC once the yeast is transferred into the fermenting tank.

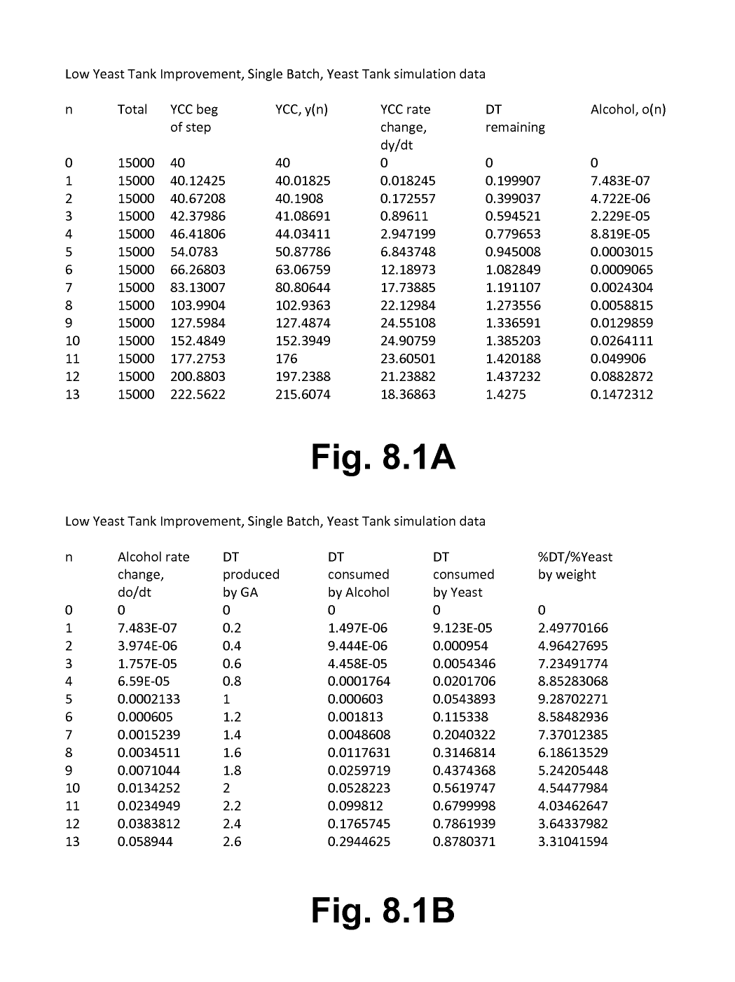

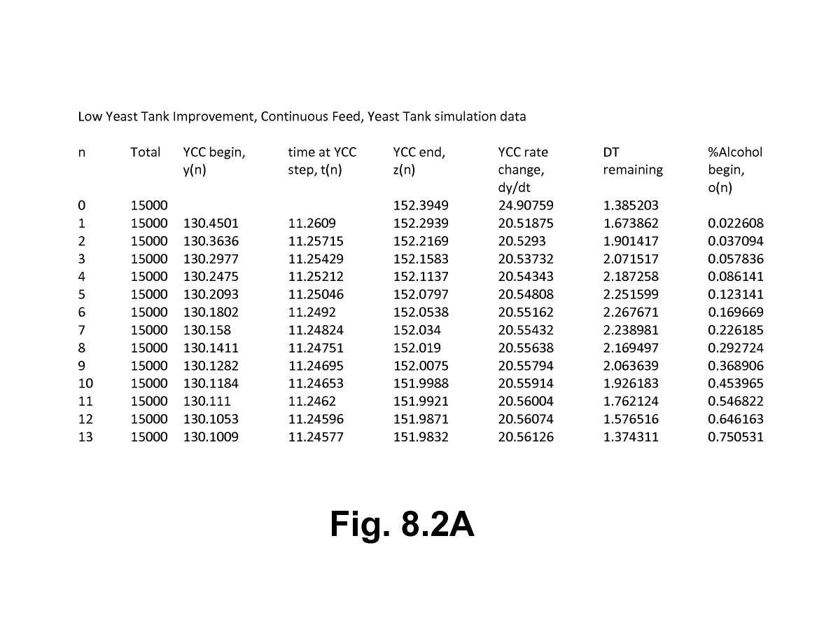

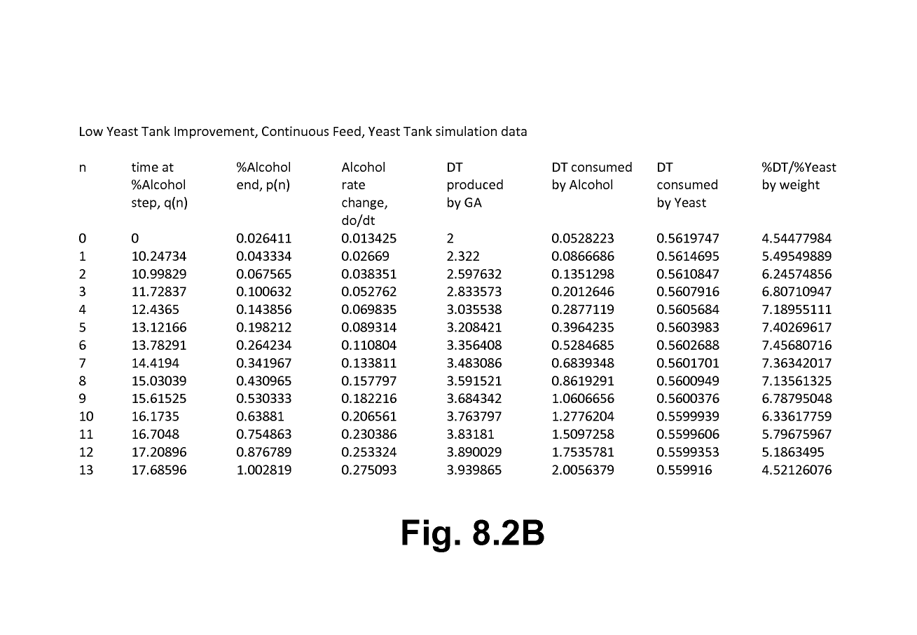

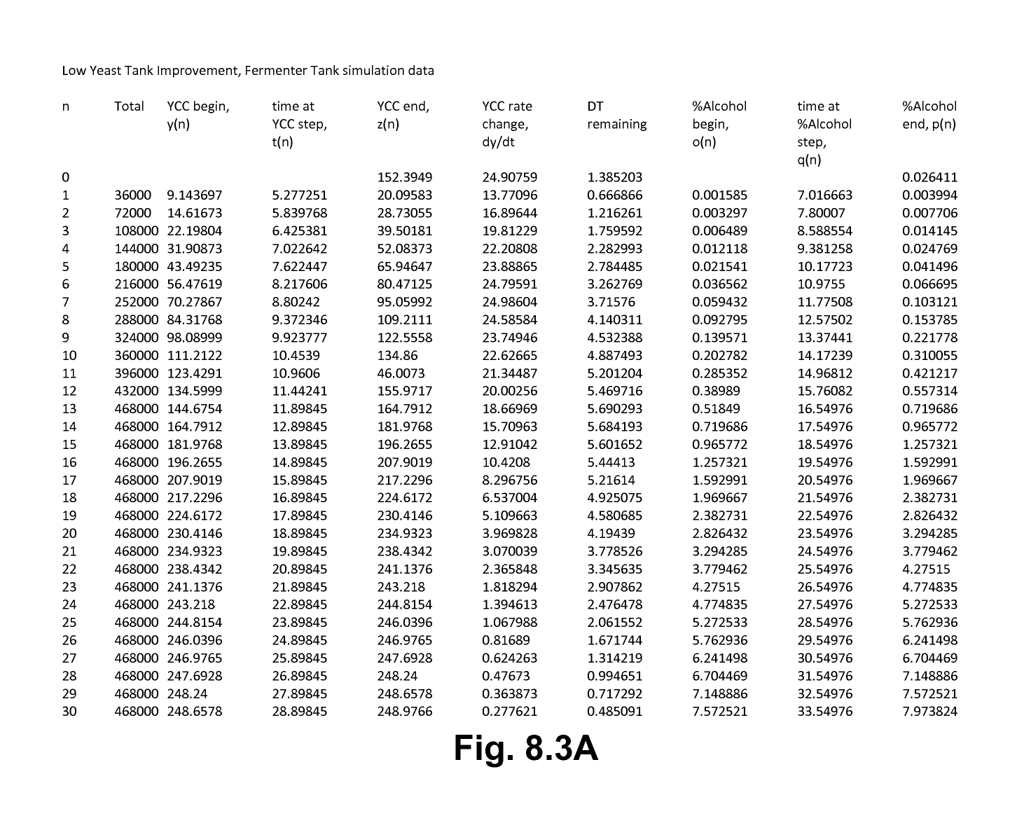

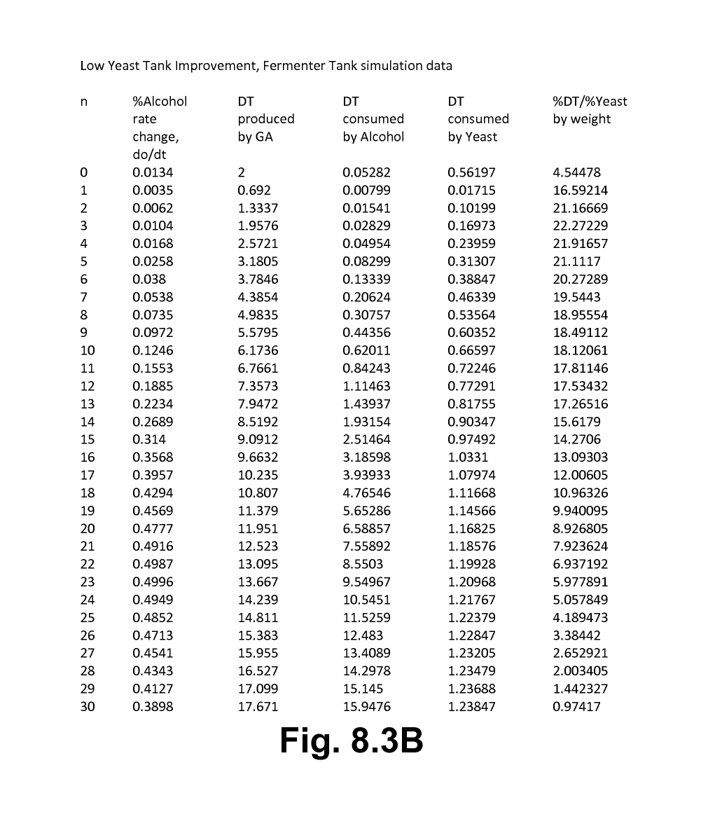

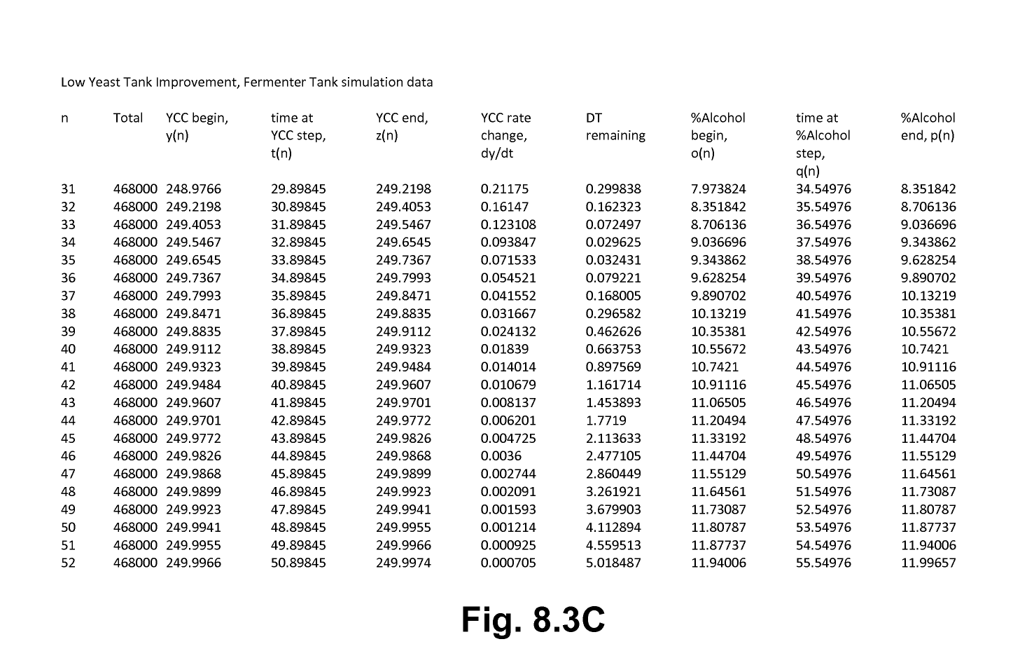

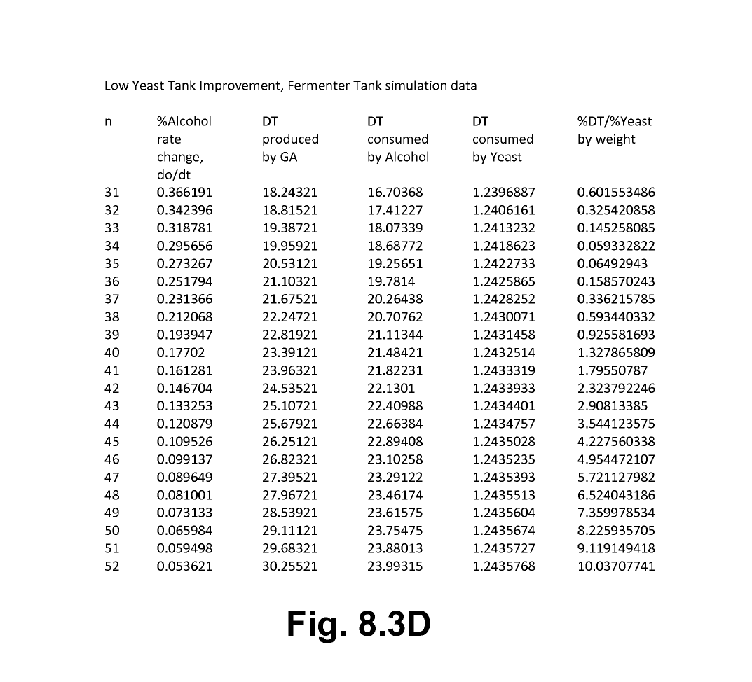

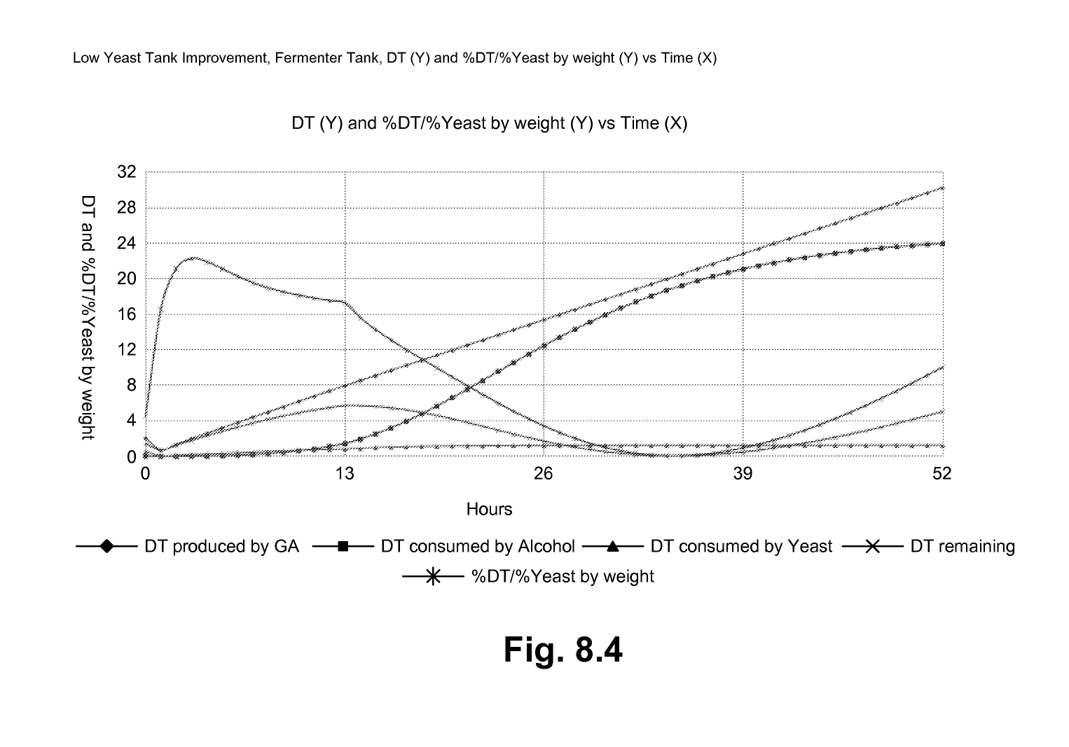

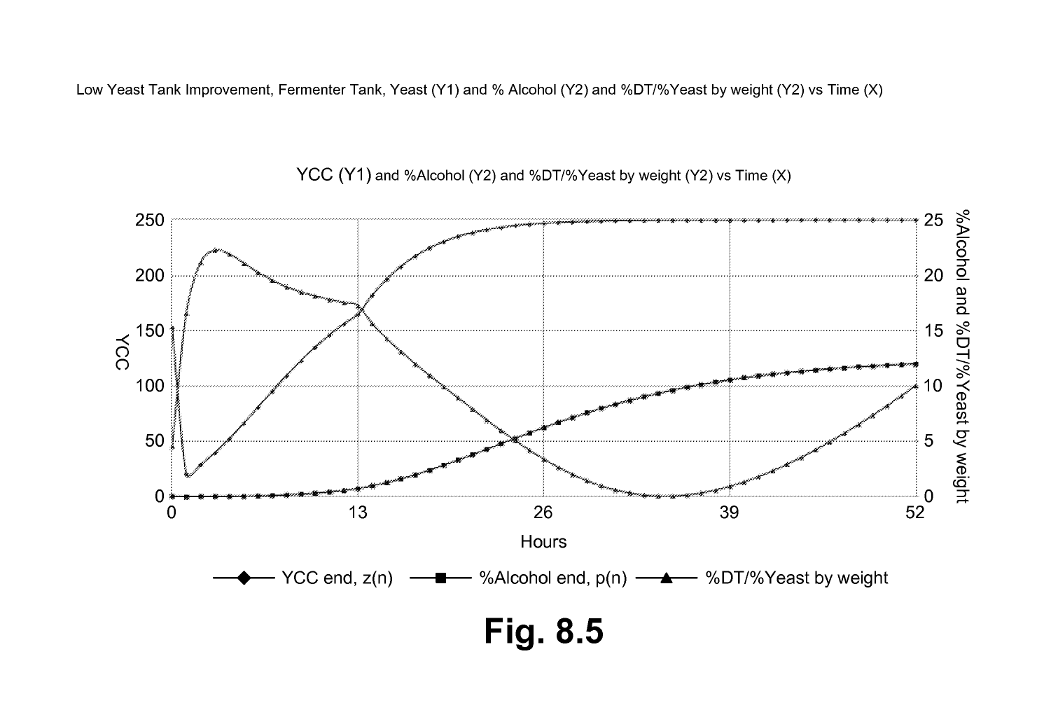

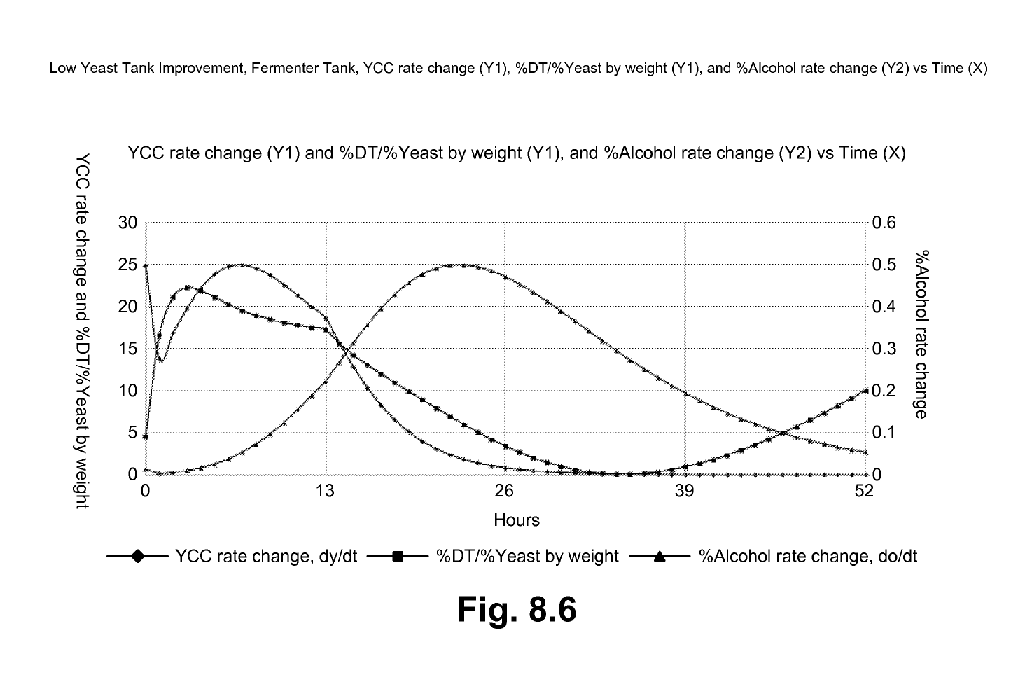

FIGS. 8.1A and 8.1B comprise a simulation data table of the Yeast Tank for the Low Yeast Tank Improvement, single batch, in accordance with some embodiments of the present invention.

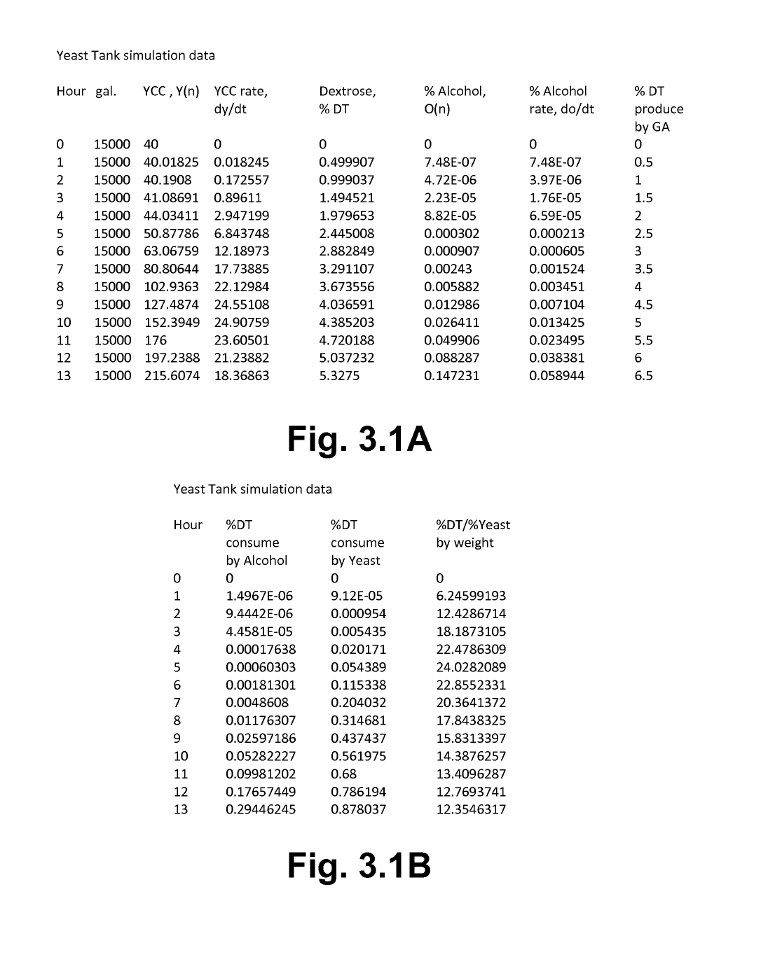

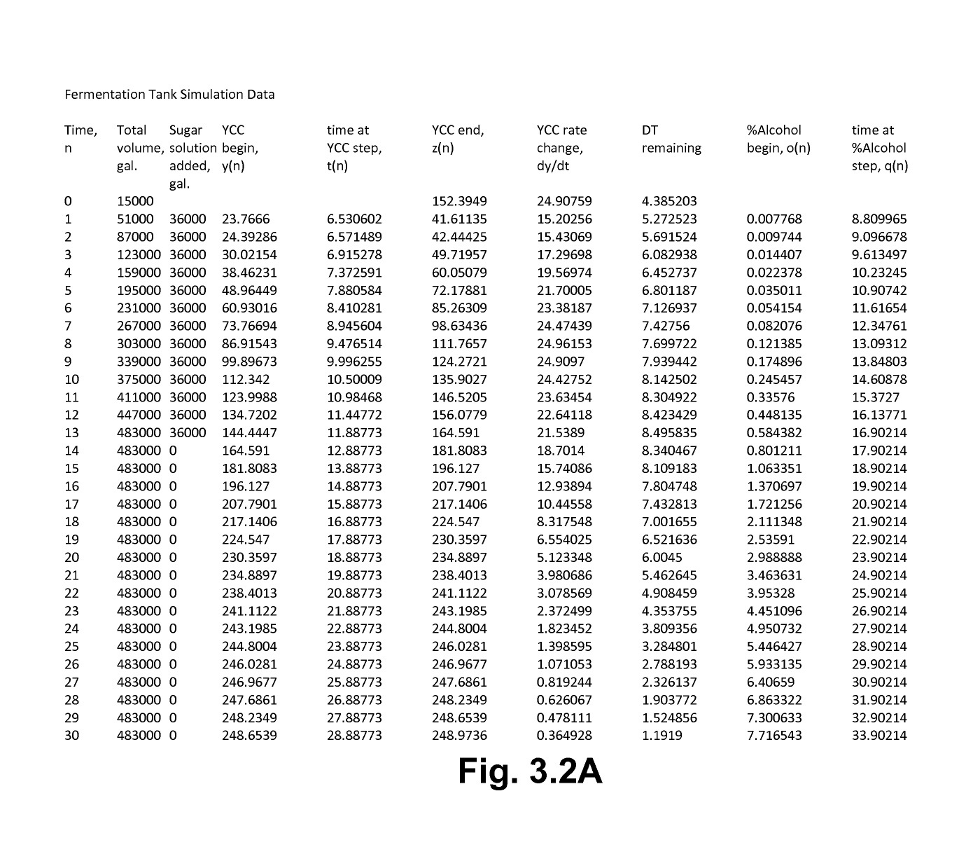

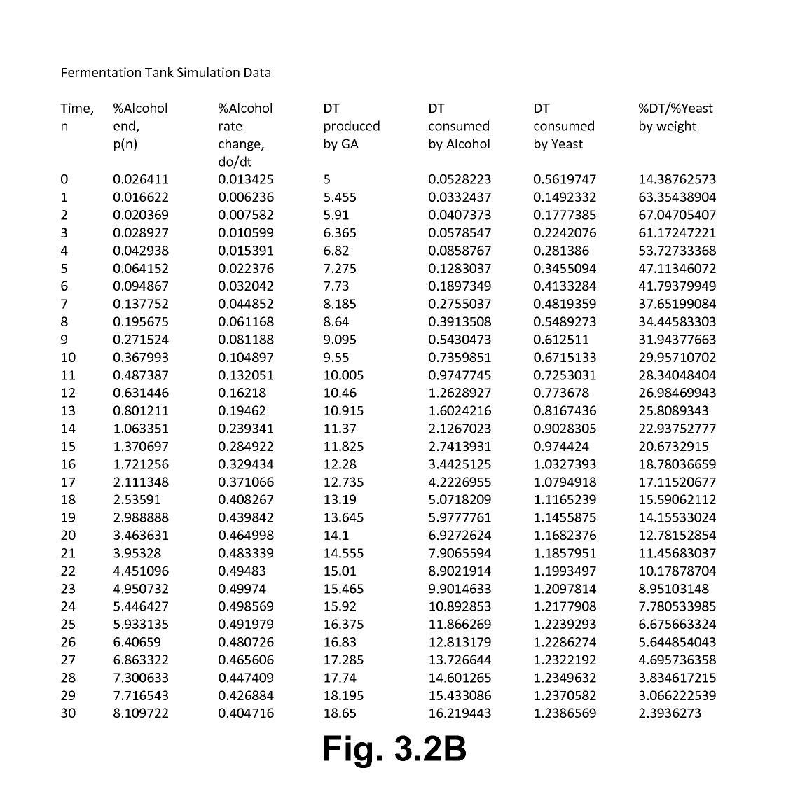

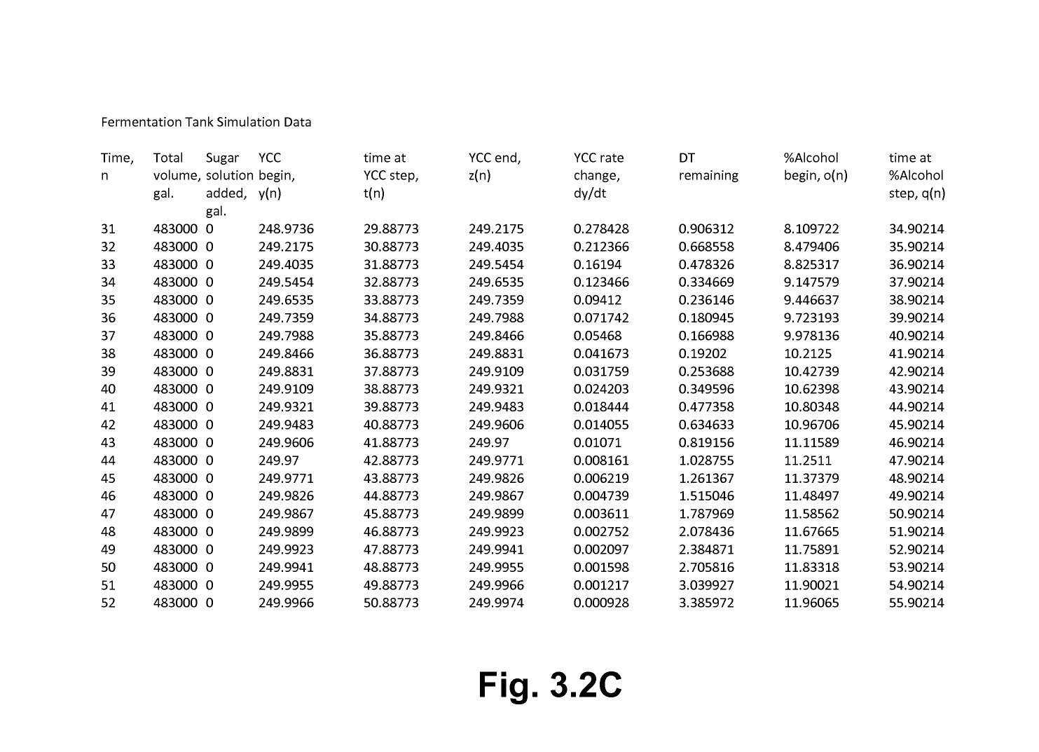

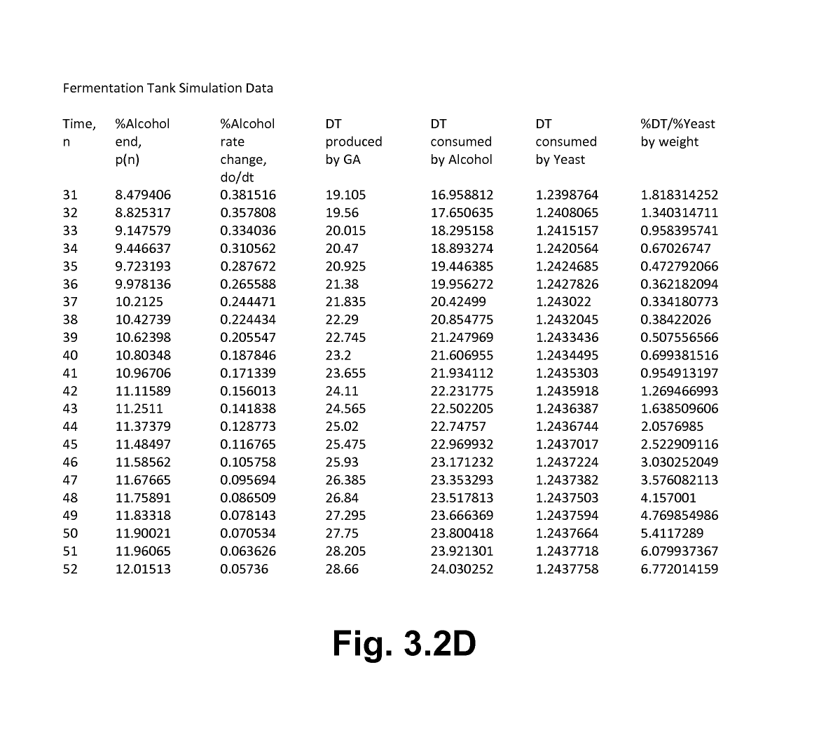

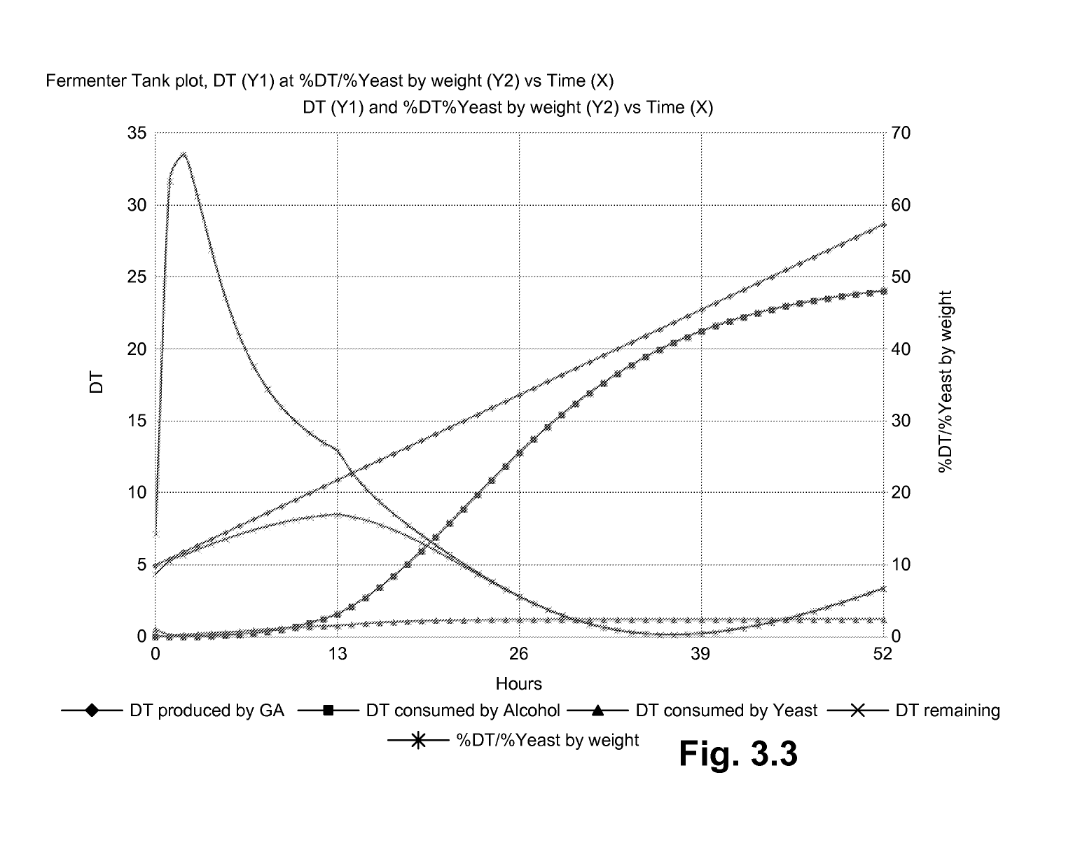

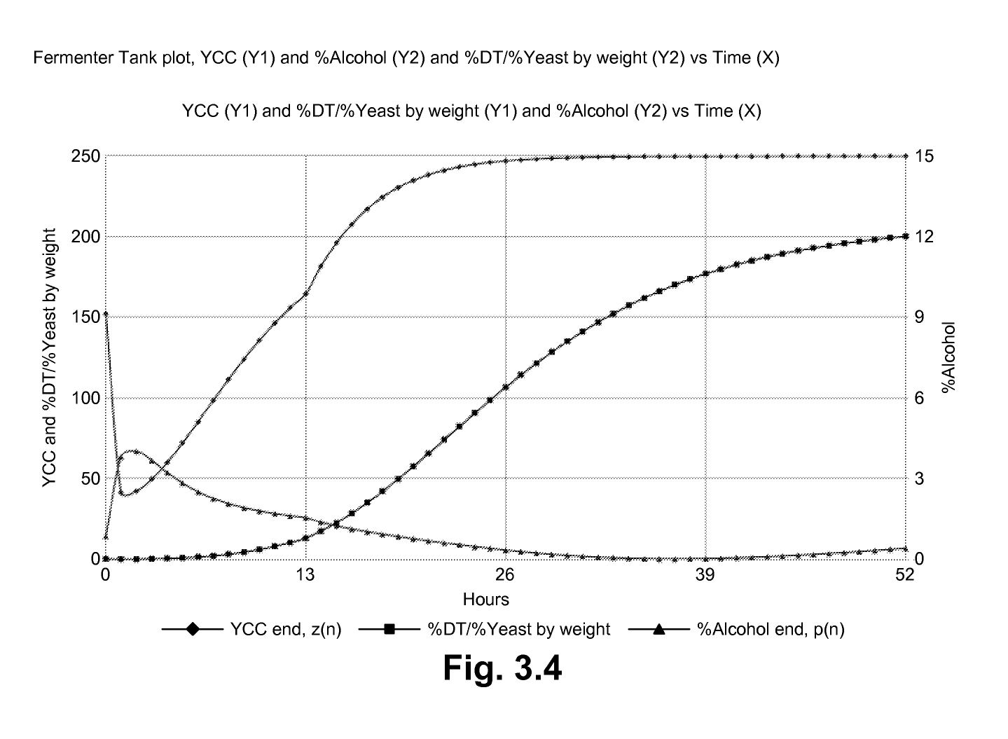

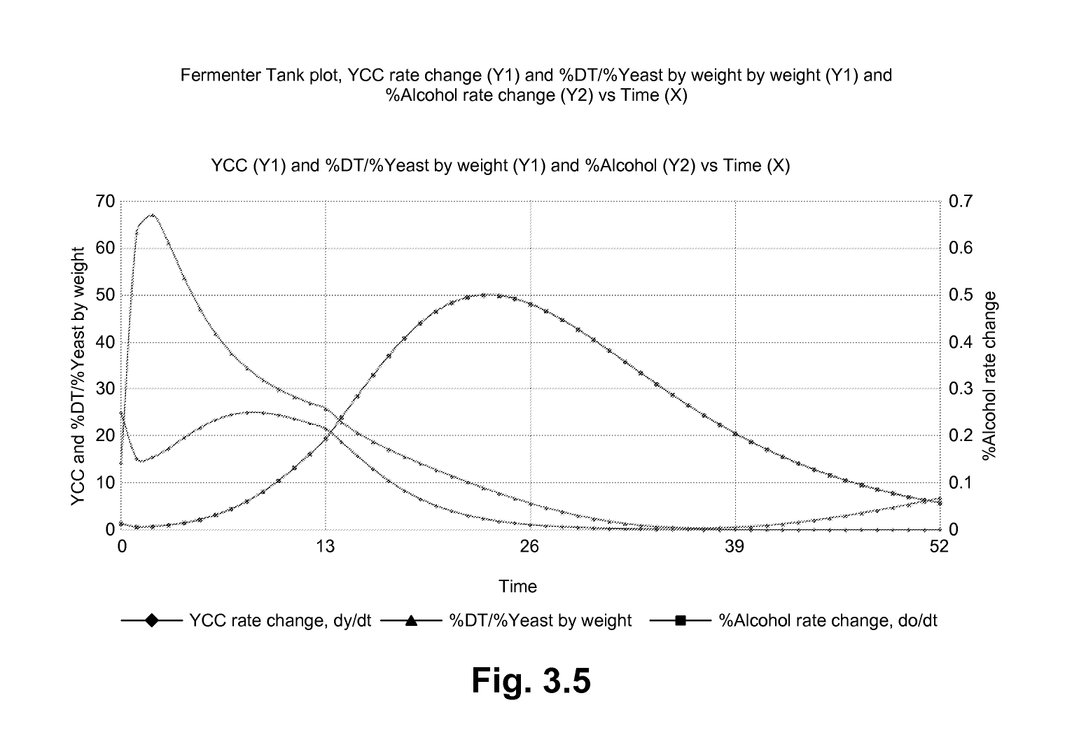

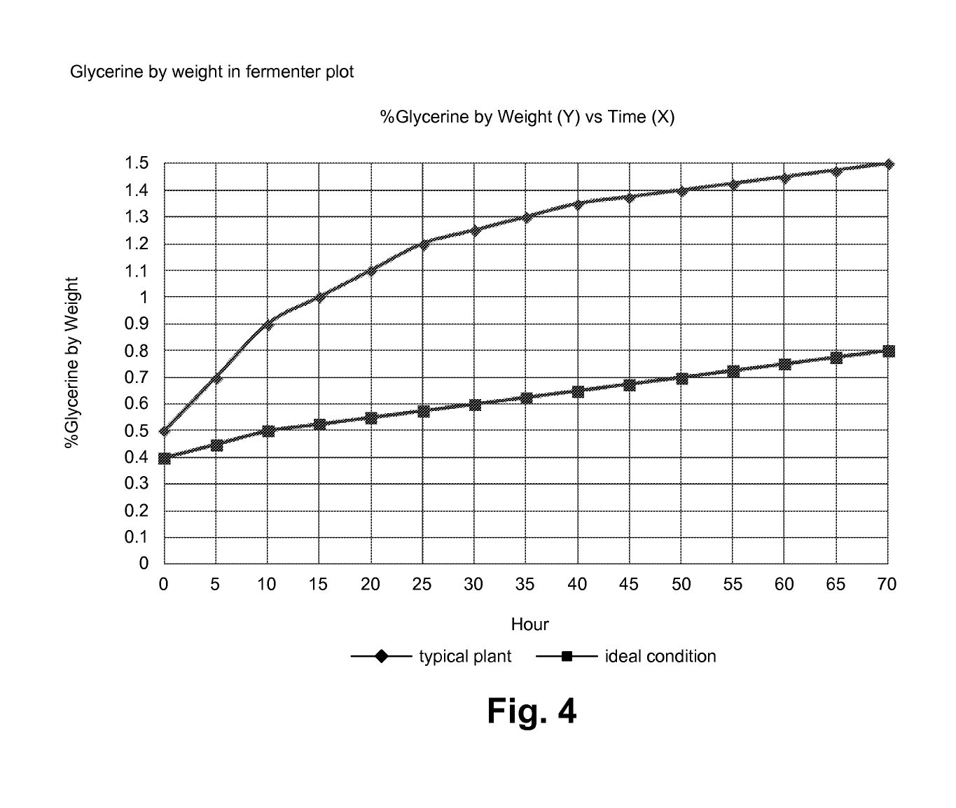

FIGS. 8.2A and 8.2B comprise a simulation data table of the Yeast Tank for the Low Yeast Tank Improvement, continuous feed, in accordance with some embodiments of the present invention. The equations used to calculate each column value for every hour is the same as FIGS. 3.2A-3.2D. A sugar solution stream of 2,160 gallons is added from hour 1 to hour 13. A computer simulation is performed taking into account how adding the fresh mash to a full yeast tank would maintain constant YCC in the yeast tank in the most active state with the optimal YCC rate change of around 20. A YCC feed of 152 is maintained consistently for the filling period cycle.

FIGS. 8.3A-8.3D comprise a simulation data table of the Fermenter Tank for the Low Yeast Tank Improvement in accordance with some embodiments of the present invention. The equations used to calculate each column value for every hour is the same as FIGS. 3.2A-3.2D. A yeast solution stream of 2,160 gallons and the remaining sugar solution stream of 33,840 gallons are sent into the fermenter tank until the tank is full. A computer simulation is performed taking into account how the two separate streams of solution affect the YCC in the fermenter tank.

FIG. 8.4 is a plot of DT (Y) and % DT/% Yeast by weight (Y) vs Time (X) from the Fermenter Tank of the Low Yeast Tank Improvement System simulation data in accordance with some embodiments of the present invention. The DT remaining reaches a maximum value of 5.69 at hour 13 with a % DT/% Yeast by weight of 17.27. This is the beginning of the rapid alcohol production fermentation period where the fermenter tank has been initially filled. The sugar is at a lower and better level so it does not convert into the byproduct Glycerine.

FIG. 8.5 is a plot of YCC (Y1) and % Alcohol (Y2) and % DT/% Yeast by weight (Y2) from the Fermenter Tank of the Low Yeast Tank Improvement System in accordance with some embodiments of the present invention. First, while the YCC from hour 0 to hour 1 in the Fermenter Tank experiences a severe drop from 152 to 20, the continuous flow of Yeast Solution allows the yeast to grow back more rapidly.

FIG. 8.6 is a plot of YCC rate change (Y1) and % DT/% Yeast by weight (Y1) and % Alcohol rate change (Y2) vs Time (X) from the Fermenter Tank of the Low Yeast Tank Improvement System simulation data in accordance with some embodiments of the present invention. First, the YCC rate change is the highest at hour 7 with a value of 24.99. In the typical system, the YCC rate change is the highest at hour 8 with a value of 24.99. The maximum YCC rate change occurs one hour earlier. Second, note that the % Alcohol rate change is the highest at hour 22 and hour 23 with a value of 0.50. In the typical system, the % Alcohol rate change is the highest at hour 23 and hour 24 with a value of 0.50. The maximum % Alcohol rate change occurs one hour earlier.

For the low yeast tank improvement system, the initial YCC at hour 0 in the 15,000 gallon yeast tank was 40. The final YCC at 10 hours in the 15,000 gallon yeast tank was 152. At a steady state, 2,160 gallons of yeast solution is fed from the yeast tank to the fermenter tank for 13 hours containing the most active YCC at 152. Therefore the yeast growth in the yeast tank is 7.11 [(152*2,160*13)/(40*15,000)] times. The expected final YCC at the end of 52 hours in the 500,000 gallon fermenter tank is 250. Therefore the yeast growth in the fermenter tank is 29.29 [(250*500,000)/(152*2,160*13)] times. Therefore the yeast has to grow 4.12 (29.29/7.11) times more in the fermenter tank than in the yeast tank. This is more manageable yeast growth for the fermenter tank allowing alcohol production to commence quicker compared to the typical system at 14.52 times. The yeast in the fermenter tank is not as stressed and more alcohol is produced. The Low Yeast Improvement System also sends 1.87 (7.11/3.80) times more yeast from the Yeast Tank to the Fermenter Tank than the typical system.

In a typical system, the % DT/% Yeast by weight reaches its highest value of 67.05 at hour 2. In the low yeast tank improvement, the % DT/% Yeast by weight reaches its highest value at 22.27 at hour 3. This is less than half the value verifying that the yeast is less stressed and able to convert more sugar solution into alcohol.

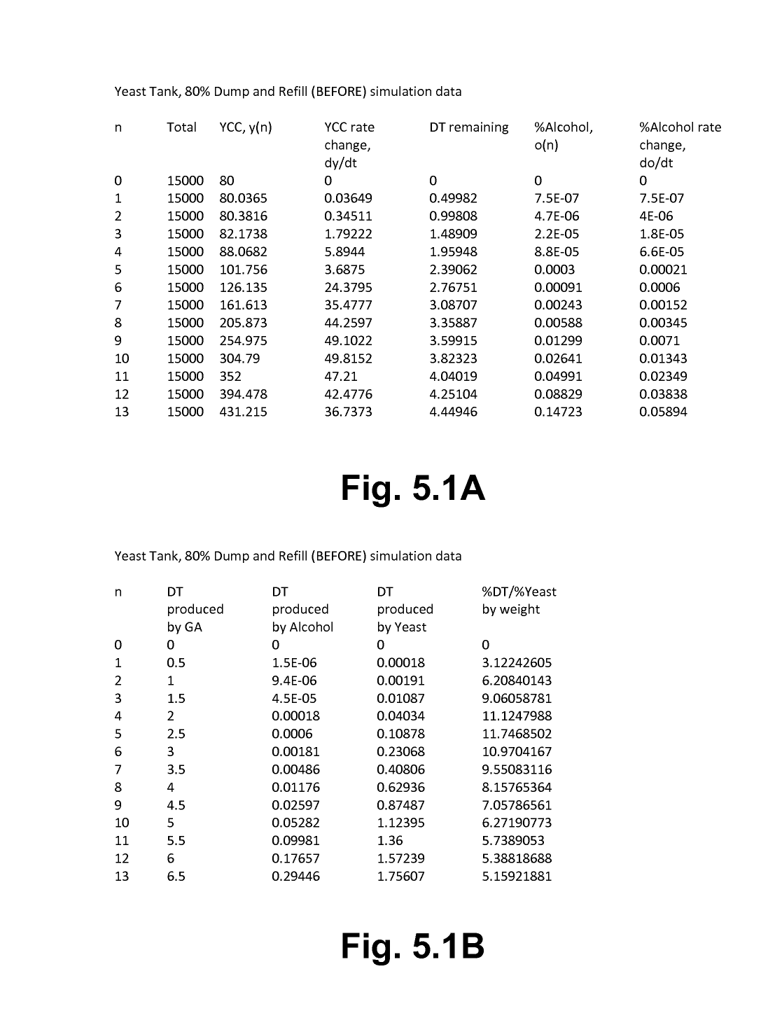

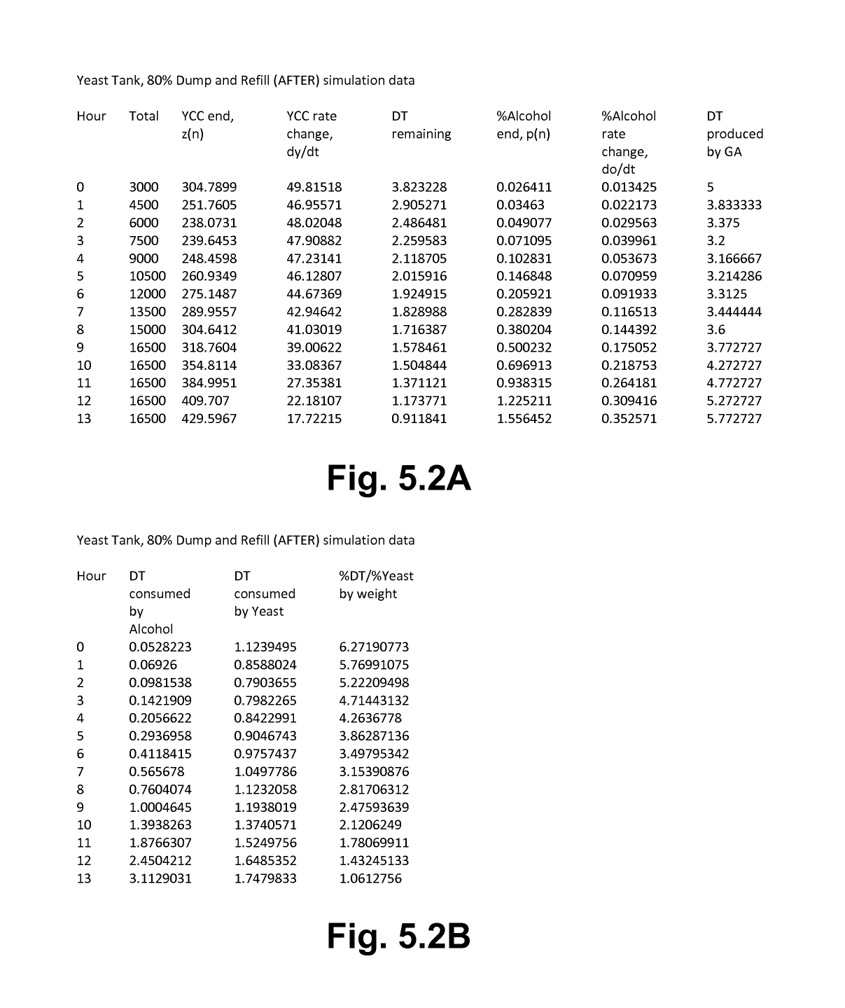

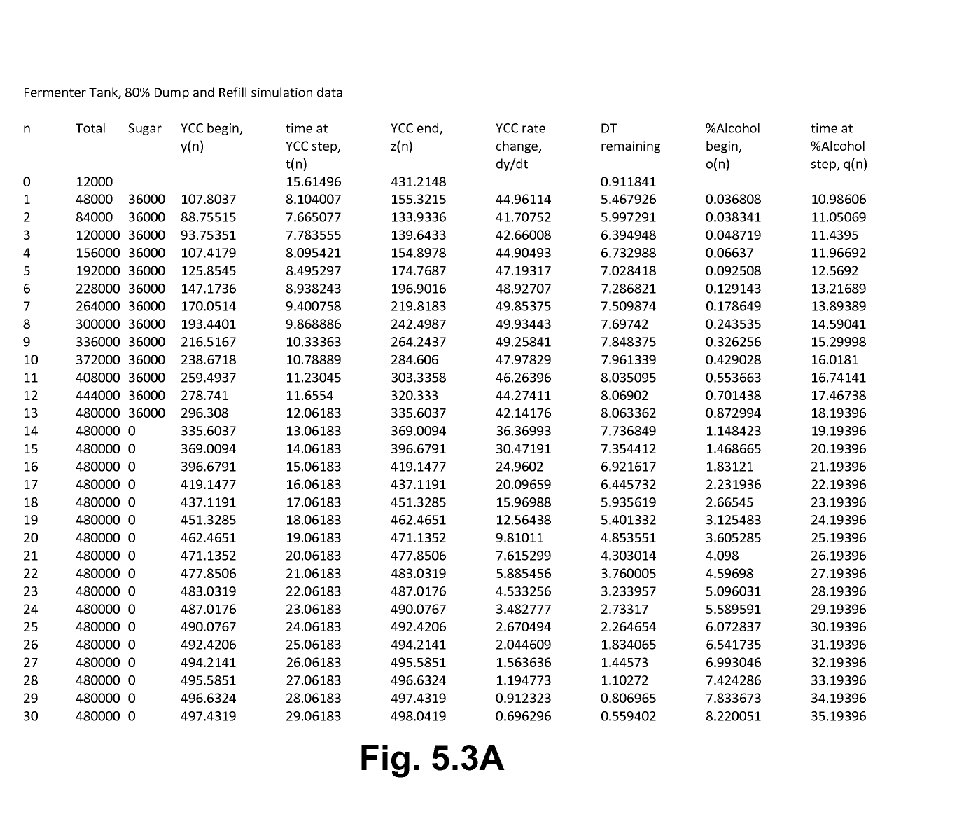

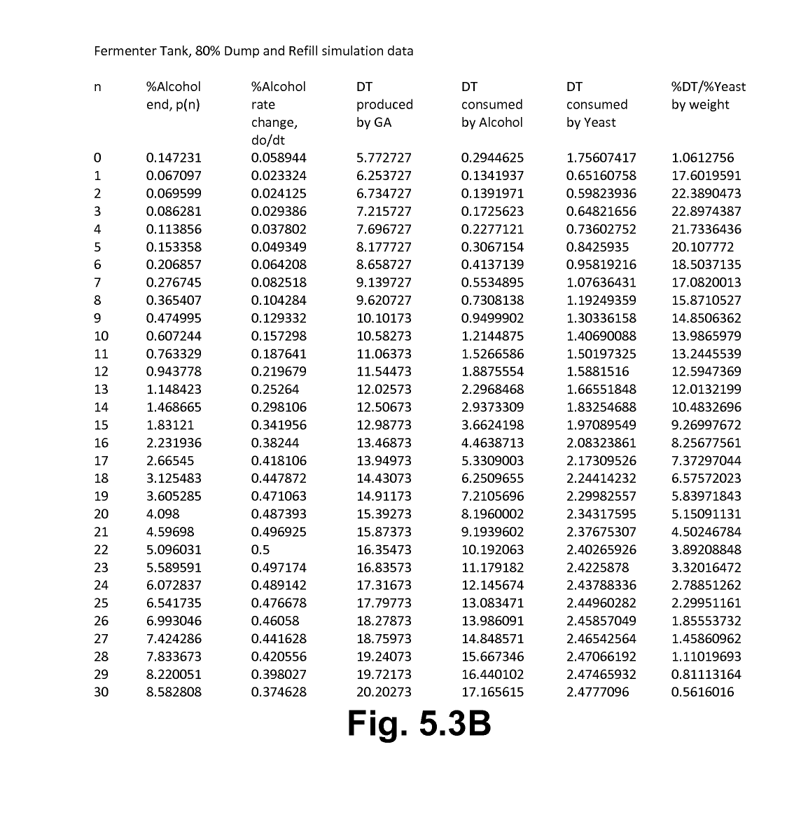

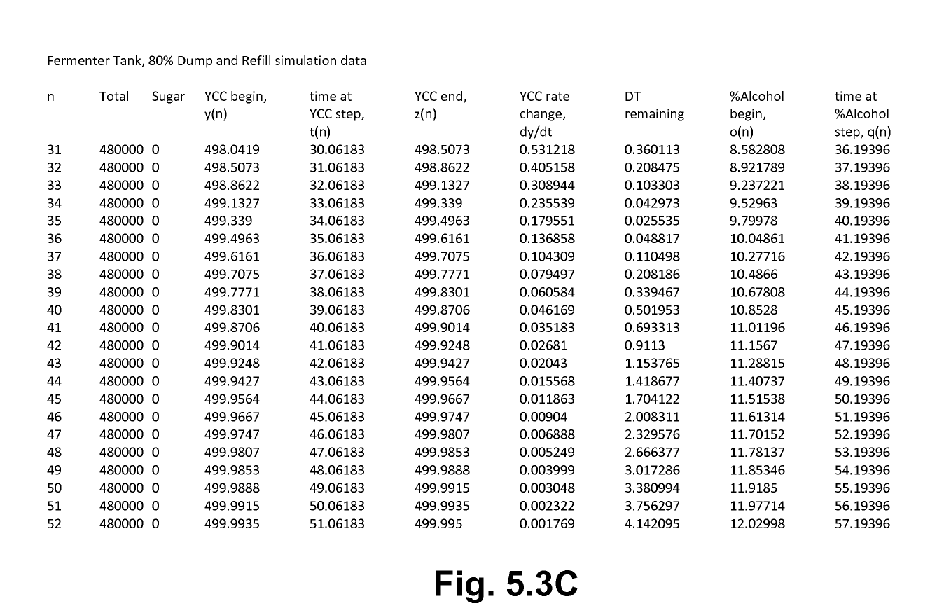

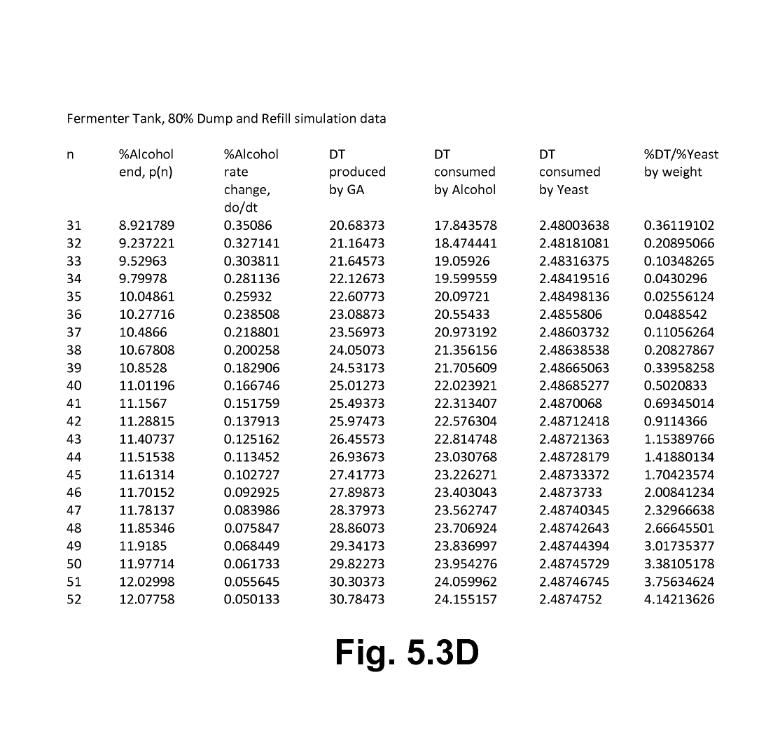

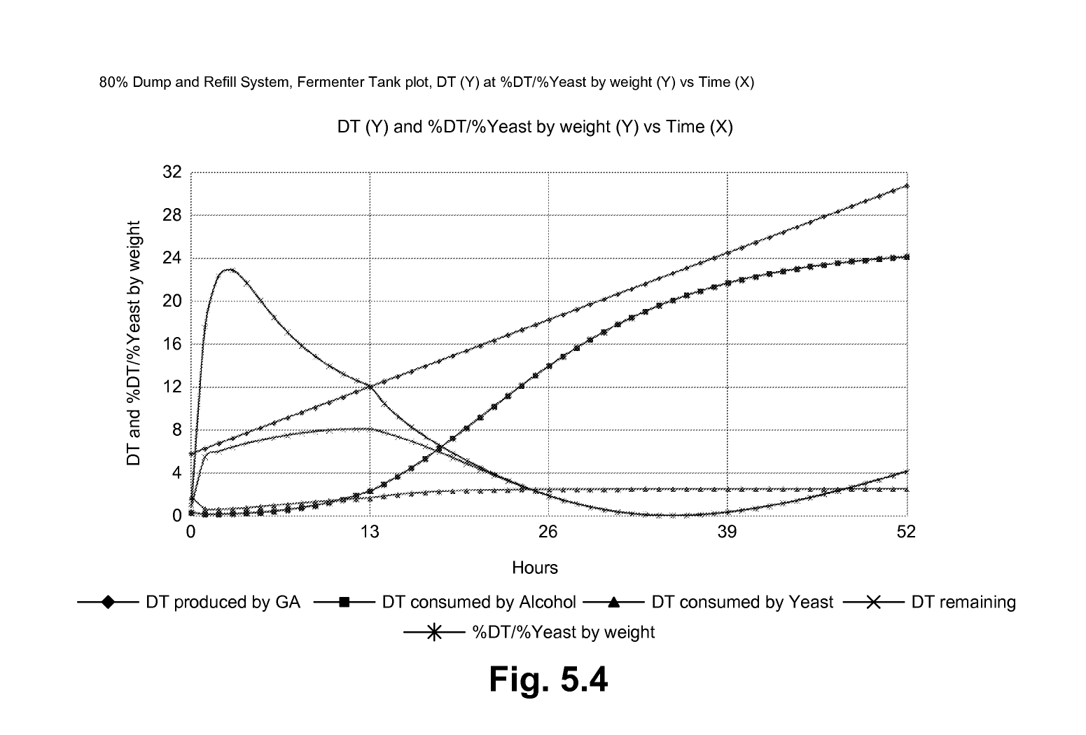

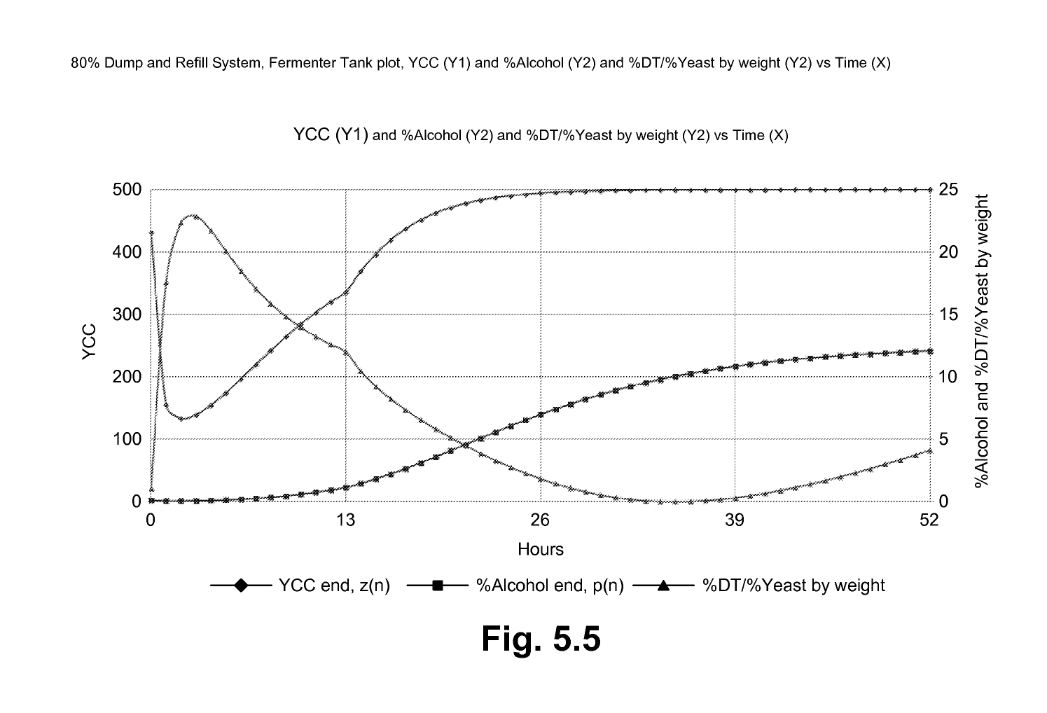

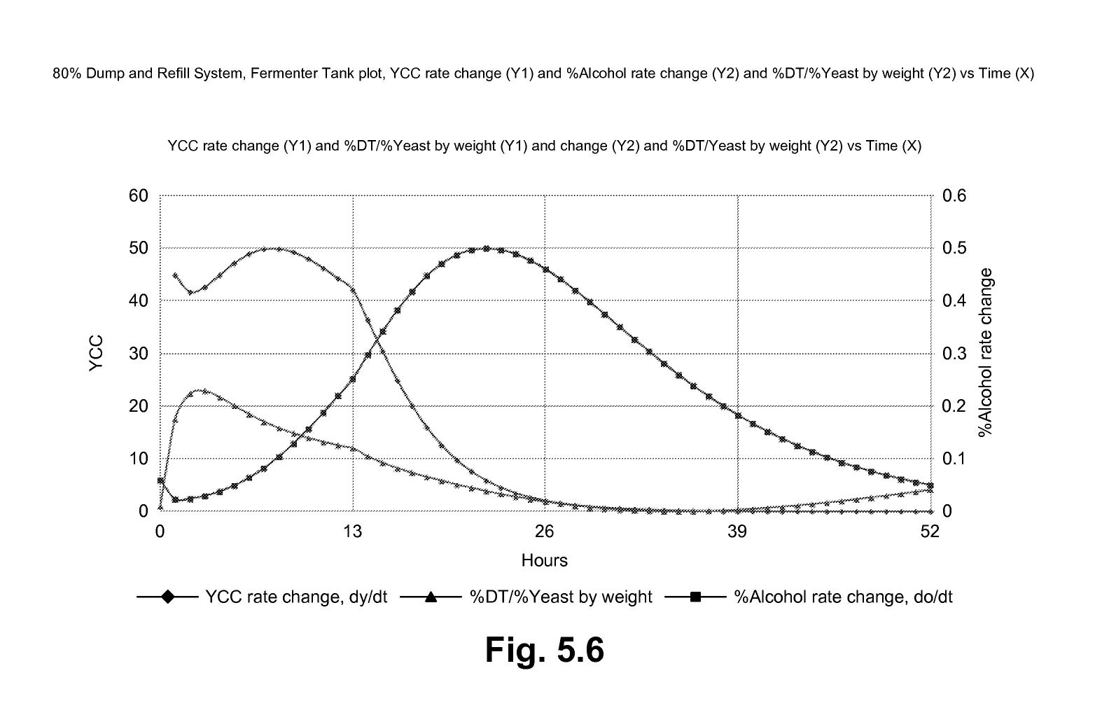

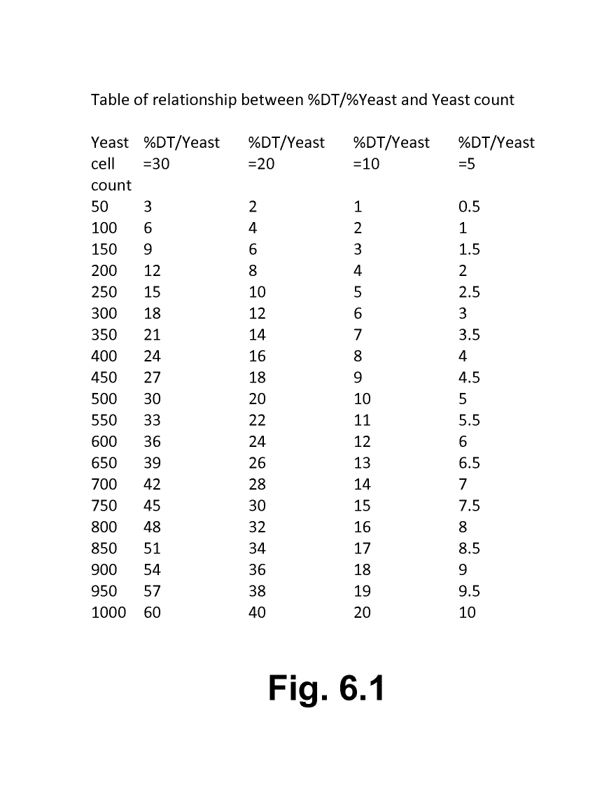

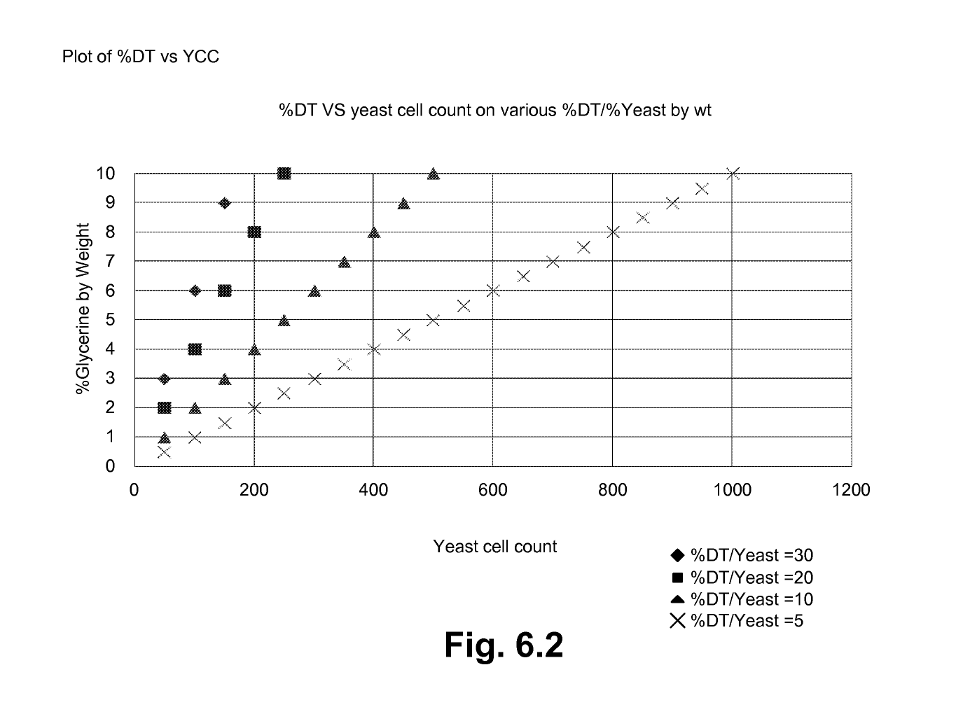

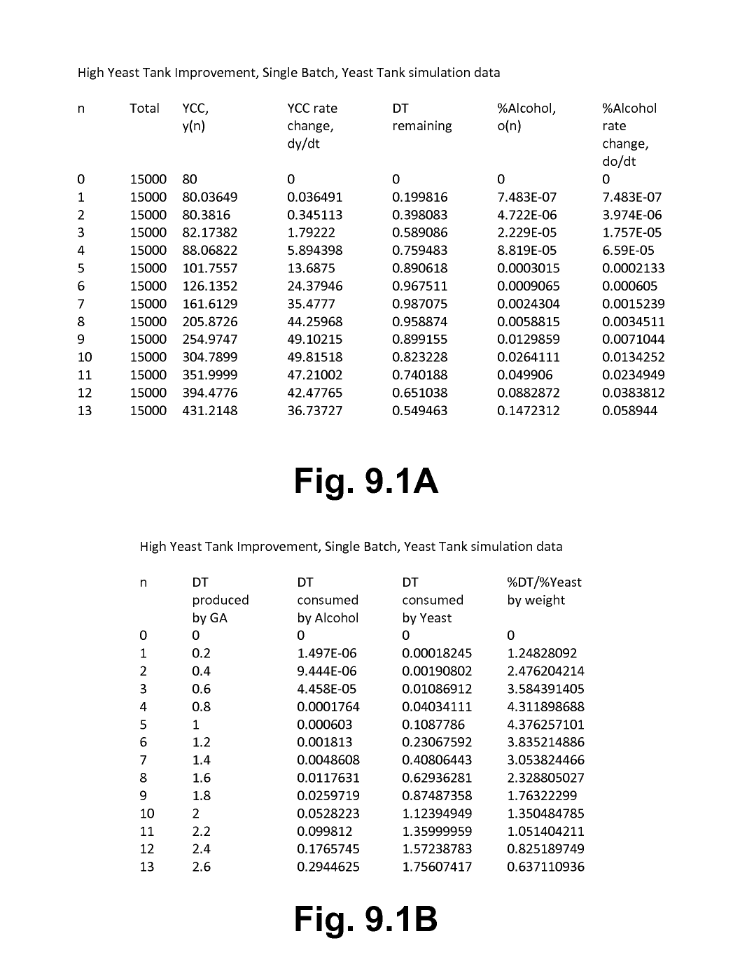

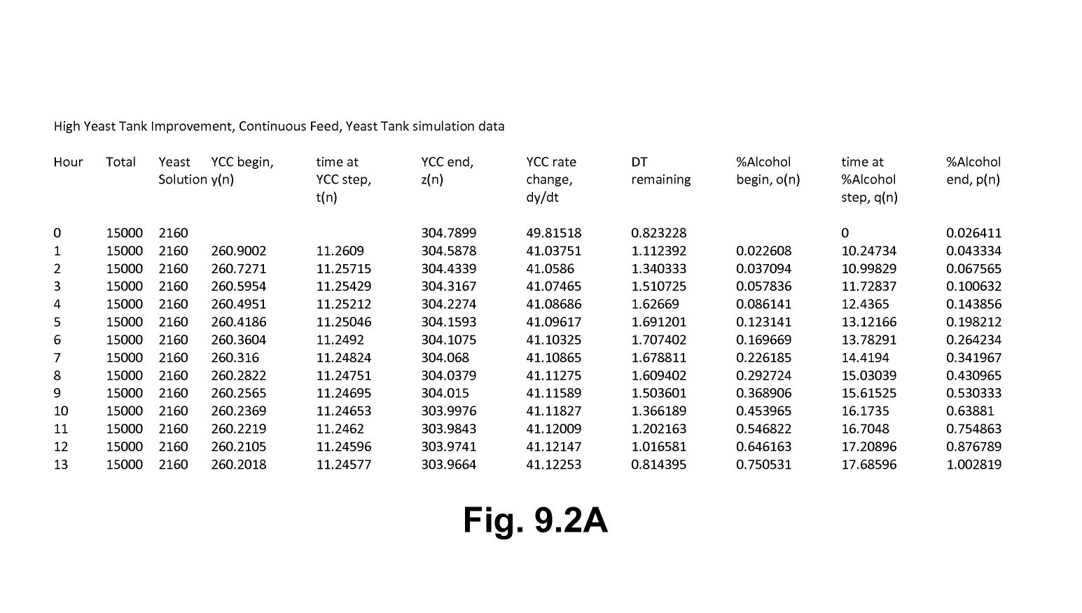

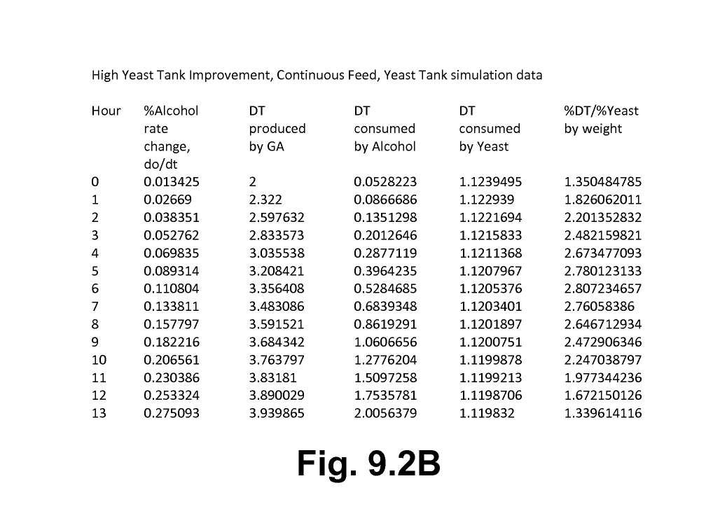

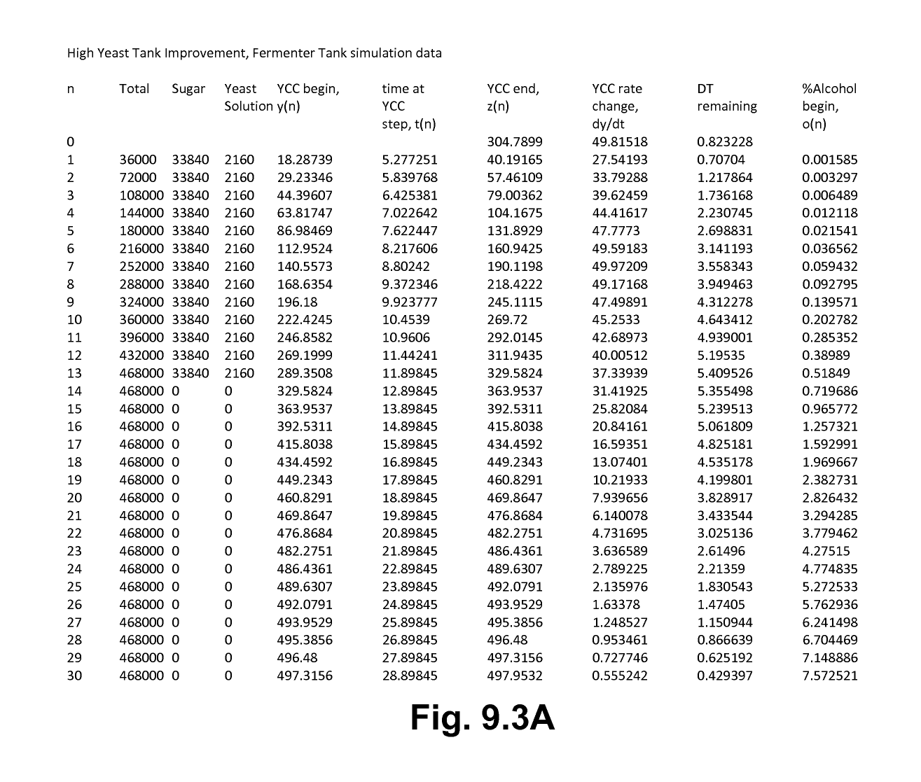

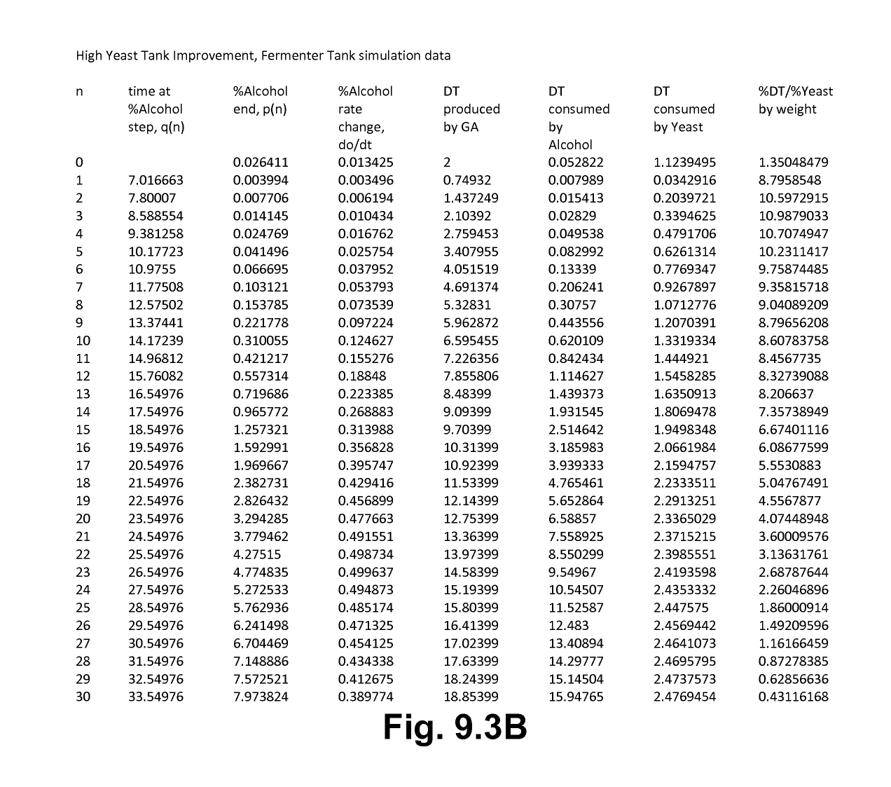

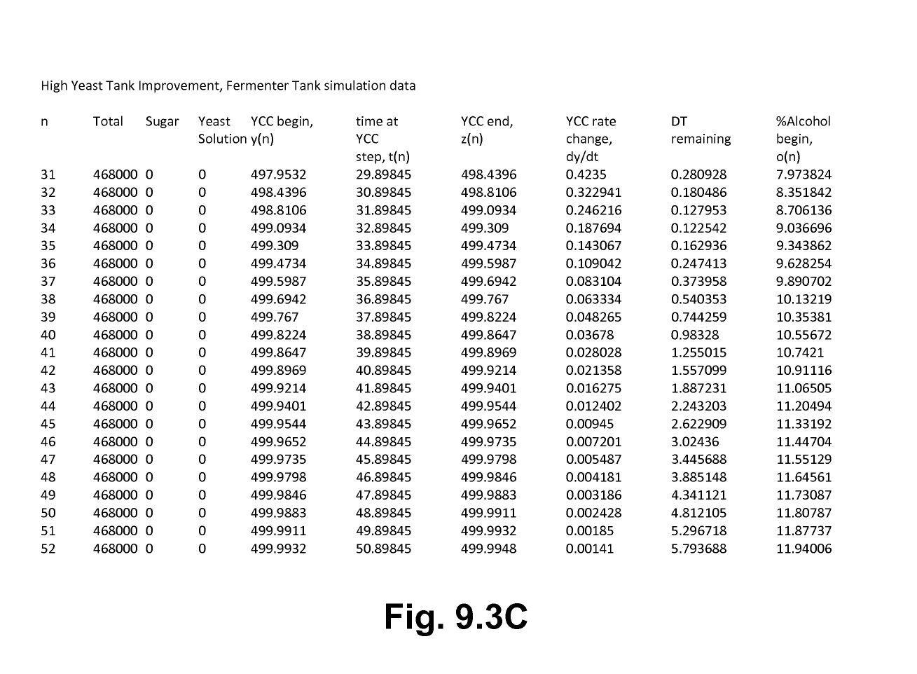

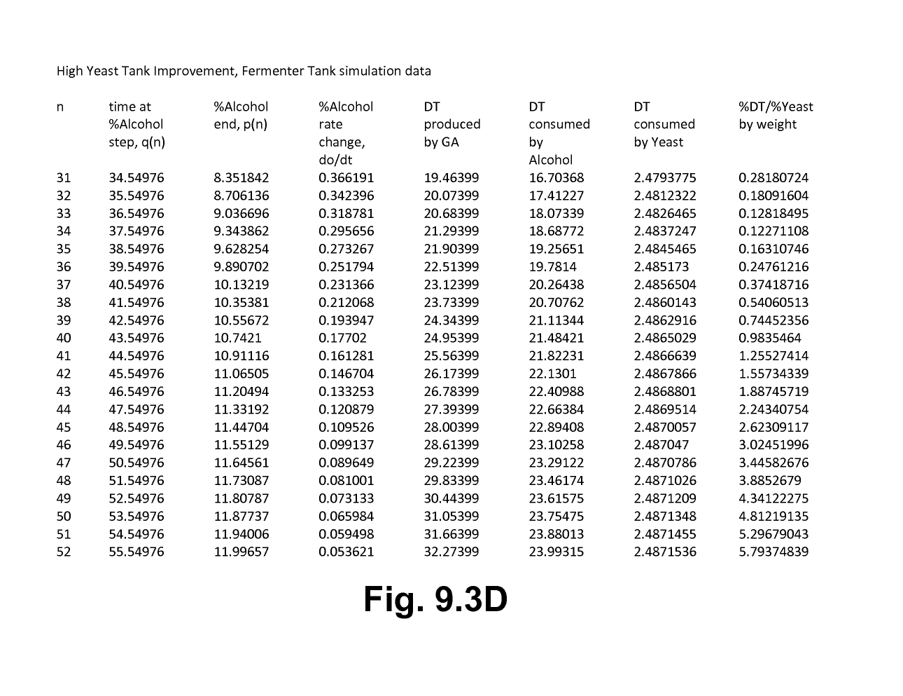

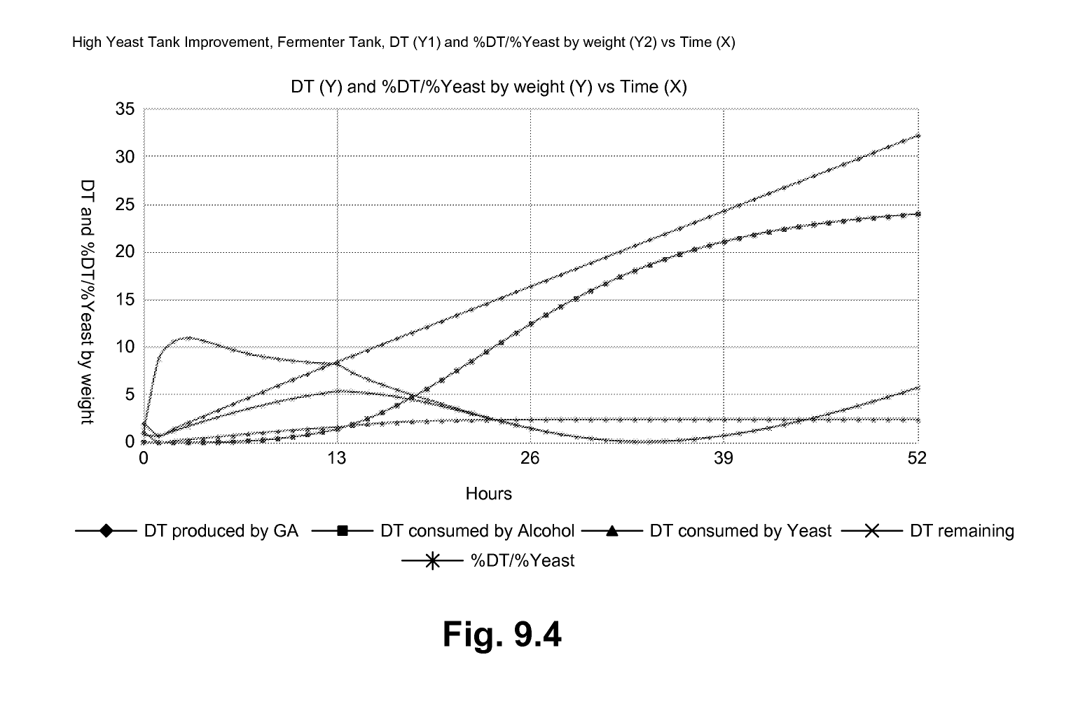

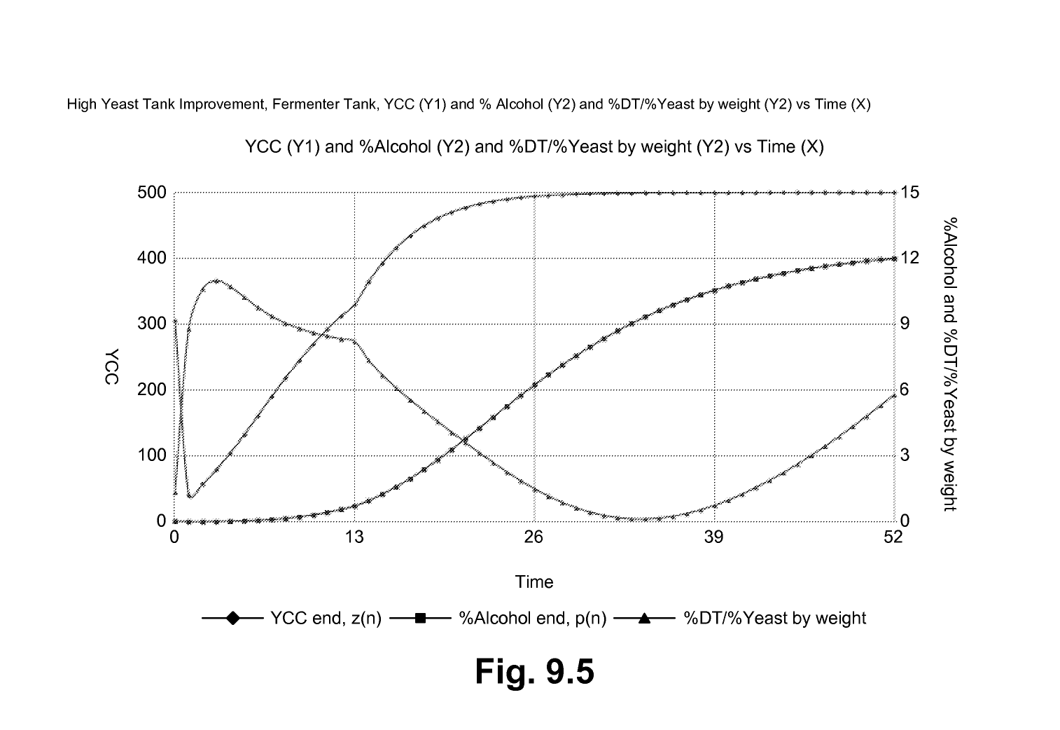

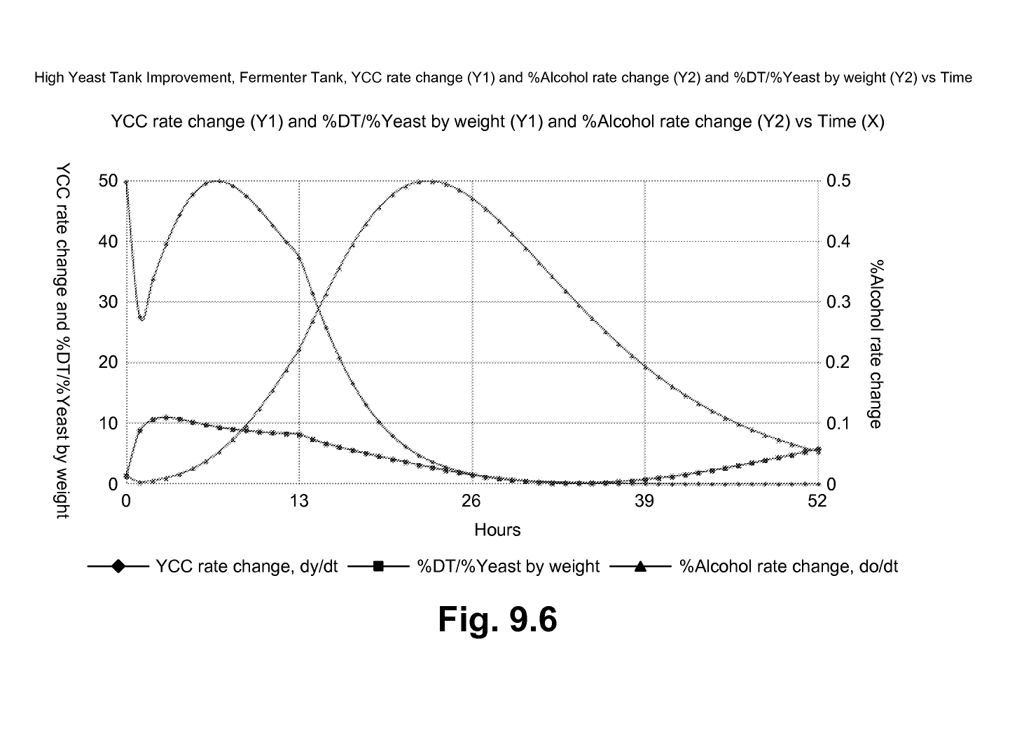

FIGS. 9.1A and 9.1B comprise a simulation data table of the Yeast Tank of the High Yeast Tank Improvement, single batch, in accordance with some embodiments of the present invention. The values of A, mu (μ), and lambda (λ) for yeast are the same 500, 50, and 6, respectively, which are the same values as the values in the FIG. 5.1. The values of A, mu (μ), and lambda (λ) for alcohol are the same 12.5, 0.5, and 18, respectively, which are as the same value as the value in the FIGS. 5.1A and 5.1B. Increasing the dosage of dry yeast in the yeast tank increases the total YCC prior to transferring into the fermenter tank, but as FIGS. 8.3A-8.3D shows it generates the same amount of Alcohol.

FIGS. 9.2A and 9.2B comprise a simulation data table of the Yeast Tank of the High Yeast Tank Improvement, continuous feed, in accordance with some embodiments of the present invention. It is the same FIGS. 8.2A and 8.2B except an YCC feed of 305 is maintained consistently for the filling period cycle.

FIGS. 9.3A-9.3D comprise a simulation data table of the Fermenter Tank of the High Yeast Tank Improvement System in accordance with some embodiments of the present invention. The remaining steam of fresh mash of 33,840 gallons is sent directly to the fermenter tank until the tank is full. A computer simulation was performed following similar analytical calculations taking into account how the two separate streams of solution affect the fermenter tank. As a result, the same amount of % Alcohol is produced in the high yeast tank Improvement (12.00%) as the low yeast tank improvement (12.00%).

FIG. 9.4 is simulation data of a plot of DT (Y1) and % DT/% Yeast by weight (Y2) vs Time (X) from the Fermenter Tank of the High Yeast Tank Improvement System in accordance with some embodiments of the present invention. Note that the DT remaining reaches a maximum value of 5.41 at hour 13 with a % DT/% Yeast by weight of 8.21 which is slightly lower than low yeast tank improvement system of 5.69 at hour 13 with % DT/% Yeast by weight of 17.27. In the typical system, the DT remaining reaches a high point value of 8.50 at hour 13 with a % DT/% Yeast by weight of 25.81. This is the beginning fermentation period where the fermenter tank is idle. The sugar is at a lower level so it does not produce as much osmotic stress resulting in excess Glycerine production.

FIG. 9.5 is simulation data of a plot of YCC (Y1) and % Alcohol (Y2) and % DT/% Yeast by weight (Y2) vs Time (X) from the Fermenter Tank of the High Yeast Tank Improvement System in accordance with some embodiments of the present invention. First, note that while the YCC from hour 0 to hour 1 in the fermenter tank experiences a severe drop from 305 to 40, the continuous flow of yeast solution allows the yeast to grow back more rapidly. Second, note that the final % Alcohol production in the low yeast tank improvement system is 12.00%.

FIG. 9.6 is simulation data of a plot of YCC rate change (Y1) and % DT/% Yeast by weight (Y1) and % Alcohol rate change (Y2) vs Time (X) from the Fermenter Tank of the High Yeast Tank Improvement System in accordance with some embodiments of the present invention. First, note that the YCC rate change is the highest at hour 7 with a value of 49.97. In the typical system, the YCC rate change is the highest at hour 8 with a value of 24.99. The maximum YCC rate change occurs one hour earlier. Second, note that the % Alcohol rate change is the highest at hour 22 and hour 23 with a value of 0.50. In the typical system, the % Alcohol rate change is the highest at hour 23 and hour 24 with a value of 0.50. The maximum % Alcohol rate change occurs one hour earlier.

For the high yeast tank improvement system, the initial YCC at hour 0 in the 15,000 gallon yeast tank was 80. The final YCC at 10 hours in the 15,000 gallon yeast tank was 305. At steady state, 2,160 gallons of yeast solution is fed from the yeast tank to the fermenter tank for 13 hours containing the most active YCC at 305. Therefore the yeast growth in the yeast tank is 7.14 [(305*2,160*13)/(80*15,000)] times. The expected final YCC at the end of 52 hours in the 500,000 gallon fermenter tank was 500. Therefore the yeast growth in the fermenter tank is 29.19 [(500*500,000)/(305*2,160*13)] times. Therefore the yeast has to grow 4.10 times (29.19/7.14) more in the Fermenter Tank than the Yeast Tank. Similar to low yeast improvement system, this is more manageable yeast growth for the fermenter tank allowing Alcohol production to commence more quickly compared to the typical system at 50.13 times. The high yeast improvement system sends 7.04 (14.28/2.02) times more yeast from the yeast tank to the fermenter tank than the typical system. The high yeast improvement system sends twice the amount of yeast from the yeast tank to the fermenter tank than low yeast improvement system.

In the typical system, the % DT/% Yeast by weight reaches its highest value of 67.05 at hour 2. In the high yeast tank improvement system, the % DT/% Yeast by weight reaches its highest value at 10.99 at hour 3. This is less than a sixth of the value verifying that the yeast is in the more ready condition to convert the sugar into alcohol.

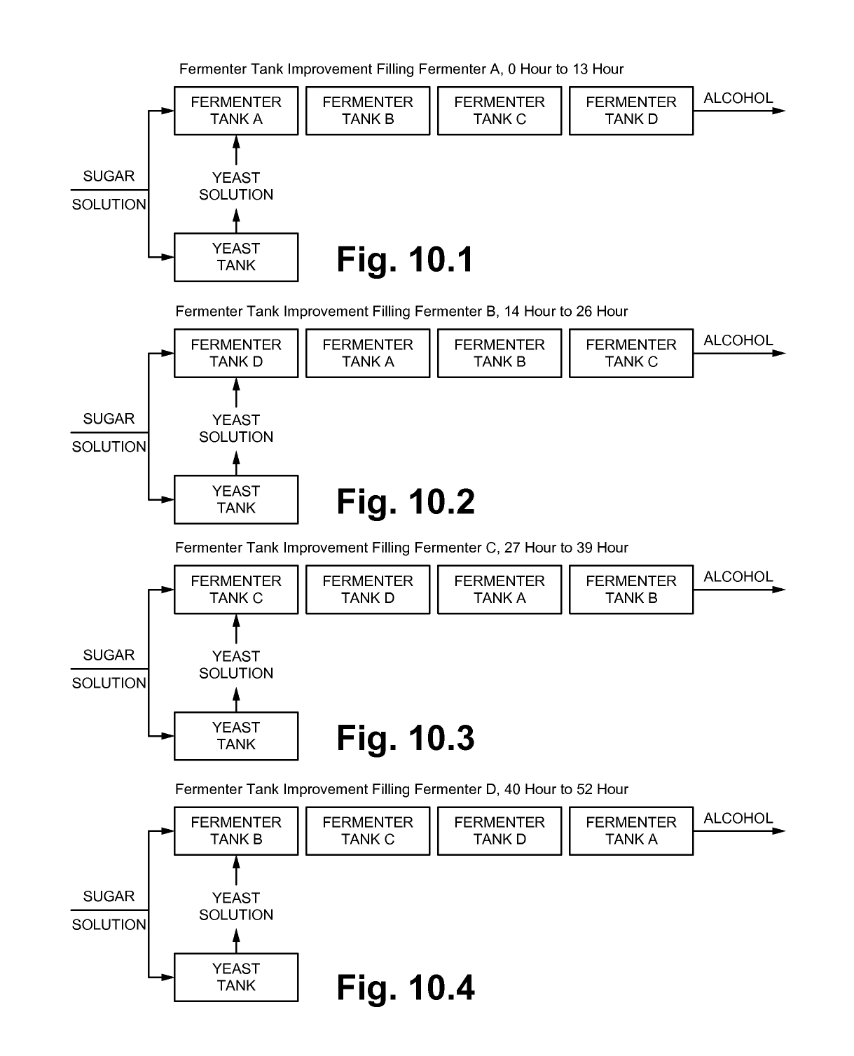

FIGS. 10.1 to 10.4 show a fermentation process flow of the Fermenter Tank Improvement System having a 52 hour cycle with 4 rotating Fermenter Tanks in accordance with some embodiments of the present invention. Note the proprietary difference between the Fermenter Tank Improvement and the typical system to analyze in this invention. The Fermenter Tank Improvement System has the Fresh Mash diverted into two streams in every cycle. The majority of the feed is sent to the filling Fermenter Tank while the remaining feed is sent to aged Fermenter Tank(s) with an approximate equal amount of Fermenter Solution sent to the filling Fermenter Tank. This alternation allows the yeast to grow stronger in the Fermenter Tank and maintain the high YCC.

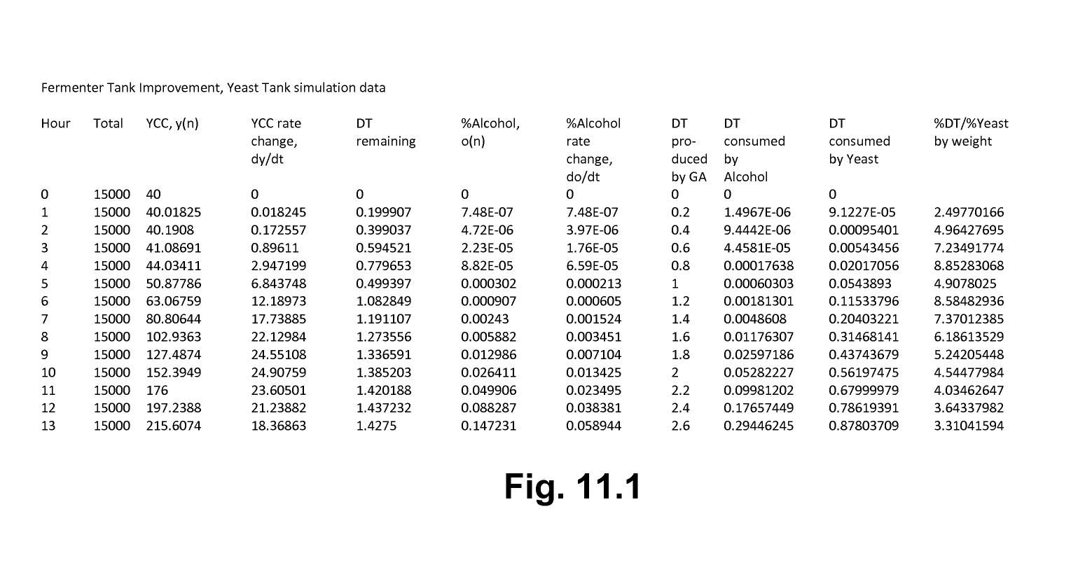

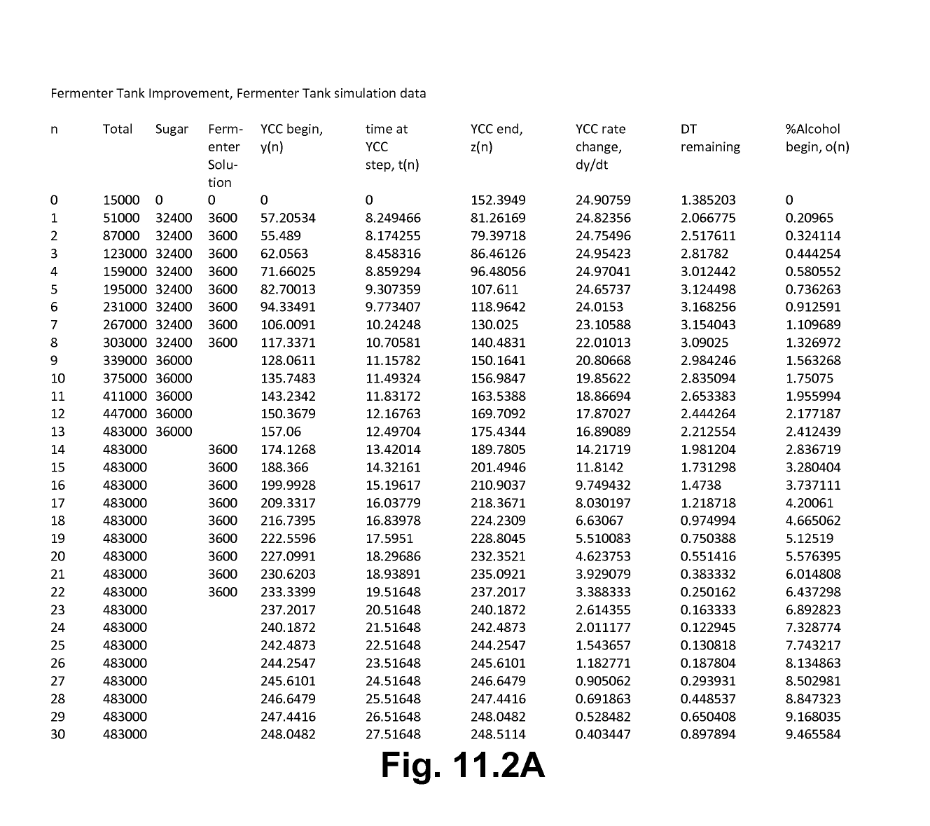

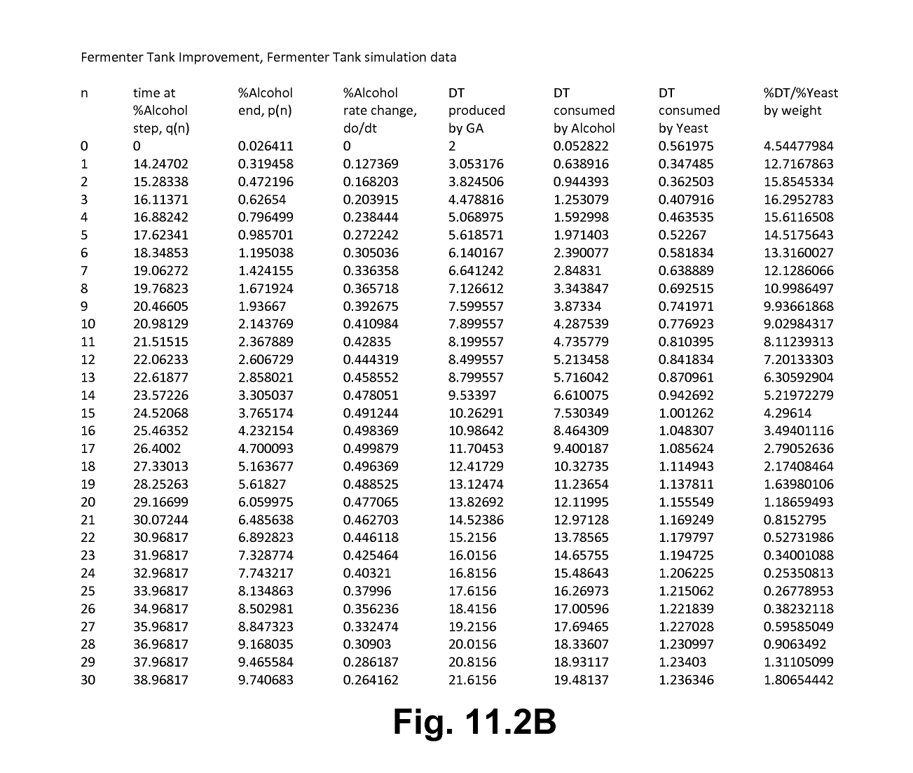

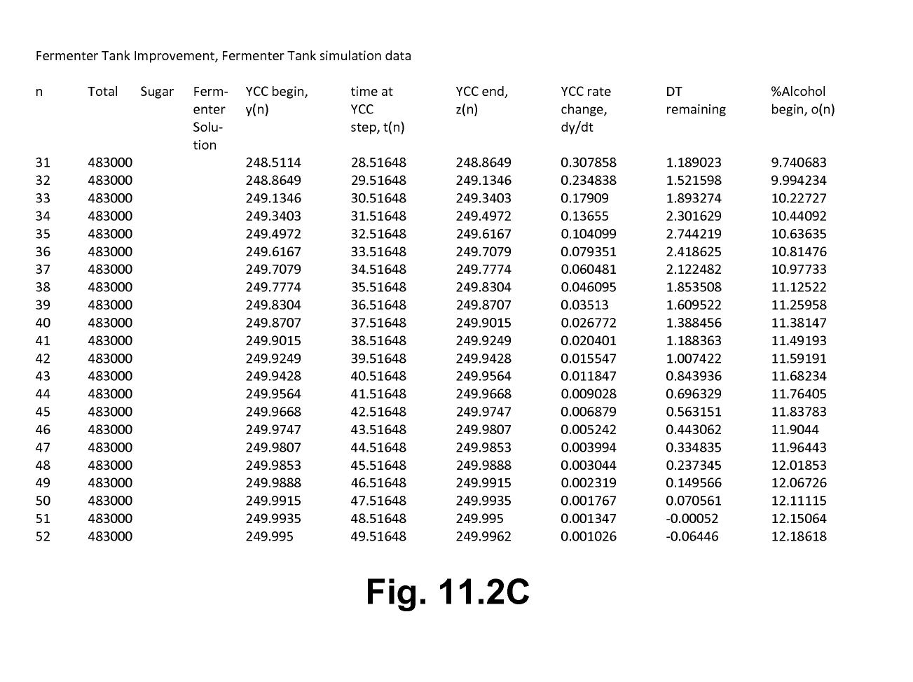

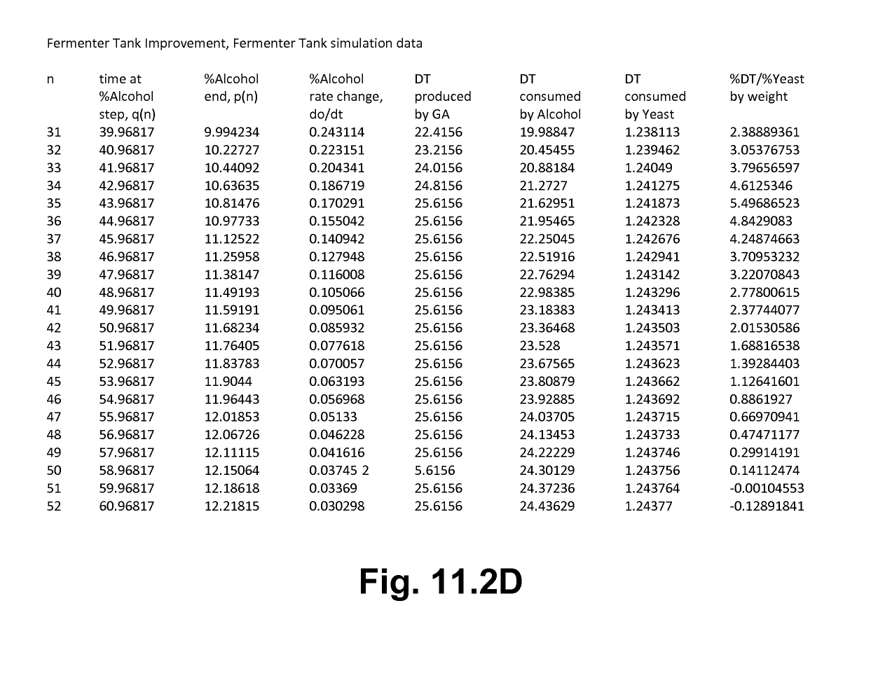

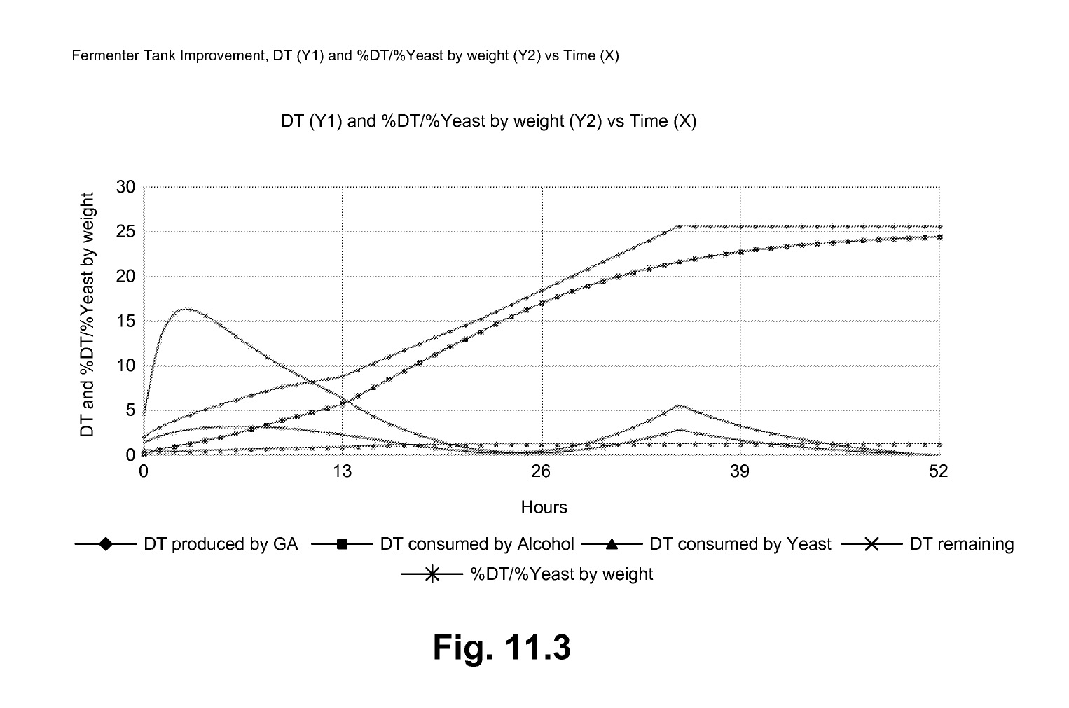

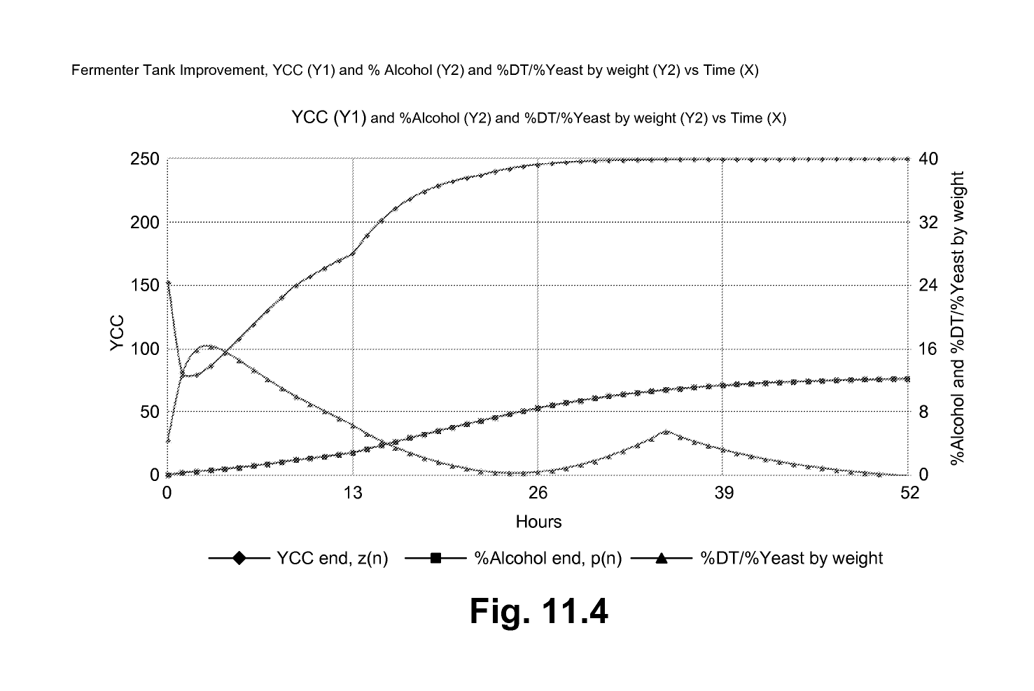

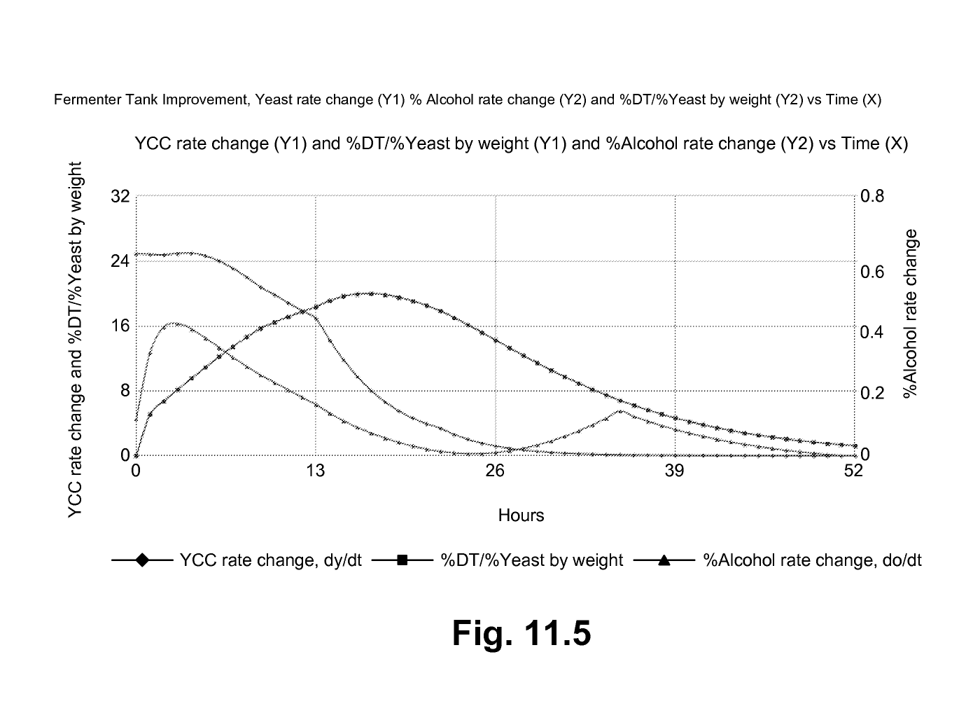

FIG. 11.1 is a simulation data table of the Yeast Tank of the Fermenter Tank Improvement System, single batch, in accordance with some embodiments of the present invention. This is the same as FIGS. 3.1A, 3.1B, 8.1A and 8.1B. The values of A, mu (μ), and lambda (λ) for yeast in Fermenter Tank Improvement System were determined to be the same 250, 25, and 6, respectively. The values of A, mu (μ), and lambda (λ) for alcohol in Fermenter Tank Improvement System were determined to be the same 12.5, 0.5, and 18, respectively.

FIGS. 11.2A-11.2D comprise a simulation data table of the Fermenter of the Fermenter Tank Improvement System in accordance with some embodiments of the present invention.

FIG. 11.3 is simulation data of a plot of DT (Y) and % DT/% Yeast by weight (Y) vs Time (X) from the Fermenter Tank of the Fermenter Tank Improvement System in accordance with some embodiments of the present invention. First, note that the % DT/% Yeast by weight increases to the maximum point at hour 3. The system then reaches close its minimum point close to 0 at hour 24. The system increases to a smaller maximum point at hour 35, then finally tapers off to 0 at hour 52. Second, note that the DT converted to Alcohol has an S-shaped curve. It continues to have a positive slope after 52 hours suggesting that not reached level shape. This indicates opportunities for improvement. The Fermenter Tank Improvement has DT remaining reach a maximum value of 3.17 at hour 6. In the typical system, the DT remaining reaches a high point value of 8.5 at hour 13. This is even better that the beginning of the fermentation period where the yeast is active and there is ample sugar to convert. The sugar is at a lower level so it does not result in excess osmotic stress to the yeast causing the production of Glycerine. The % DT/% Yeast by weight is much lower as the highest point is 16.30 at hour 3 for Fermenter Tank Improvement compared to 67.05 at hour 2 for the Current System.

FIG. 11.4 comprises simulation data of a plot of YCC (Y1) and % Alcohol (Y2) and % DT/% Yeast by weight (Y2) vs Time (X) from the Fermenter Tank of the Fermenter Tank Improvement System in accordance with some embodiments of the present invention. While the YCC from hour 0 to hour 1 in the Fermenter Tank experiences a severe drop from 150 to 80, the continuous flow of yeast solution allows the yeast to grow back more rapidly. The Fermenter Tank Improvement System outpaces the Current System yeast growth throughout the 52 hour cycle. Alcohol production in the Fermenter Tank Improvement System begins immediately at hour 3 as opposed to hour 6 in the Current System. As a result, the Alcohol production in the Fermenter Tank Improvement System at 12.22% is much higher than the Current System at 12.02%.

FIG. 11.5 comprises simulation data of a plot of YCC rate change (Y1) and % DT/% Yeast by weight (Y1) and % Alcohol rate change (Y2) vs Time (X) from the Fermenter Tank of the Fermenter Tank Improvement System in accordance with some embodiments of the present invention. First, note that the Yeast rate change stays consistently in the 20 to 25 range during hour 0 to hour 9 of the two flow stage with a high value of 24.97 at hour 4. This indicates that strong yeast is rapidly growing. Second, note that the Alcohol production rate change stays consistently in the 0.45% to 0.50% range during hour 14 to hour 22 of the Aged Fermenter Tank flow stage with the highest values of 0.50 at hour 16 and hour 17. The system has more ideal alcohol production conditions between hour 15 and hour 25. As a result more Alcohol has been produced. The maximum Alcohol production rate for the Fermenter Tank Improvement System occurs at hour 16 and hour 17 as opposed to the hour 25 for the Current System.

For the Fermenter Tank Improvement System, the initial YCC at hour 0 in the 15,000 gallon Yeast Tank was 40. The final YCC at 10 hours in the 15,000 gallon Yeast Tank was 152. Therefore the yeast growth in the Yeast Tank was 3.80 (152/40) times as Current System. At steady state, 3,600 gallons of Fermenter Solution was fed from an Aged Fermenter Tank to the newly filling Fermenter Tank for the first 9 hours containing the most active yeast with YCC at 190, 201, 211, 218, 224, 229, 232, 235, and 237. The average of the YCC for the first 9 hours is 220 [(190+201+211+218+224+229+232+235+237)/9]. Therefore the yeast sent from the Aged Fermenter Tank to the filled Fermenter Tank is 11.88 [(3,600*9*220)/(15,000*40) times more than the Current System. Therefore the total yeast sent to the Fermenter Tank is 15.68 (3.80+11.88) times more than the Current System during the 13 hour filling cycle. Therefore the yeast sent to the Fermenter Tank over the entire fermentation is 4.12 (15.68/3.80) times more than the Current System.

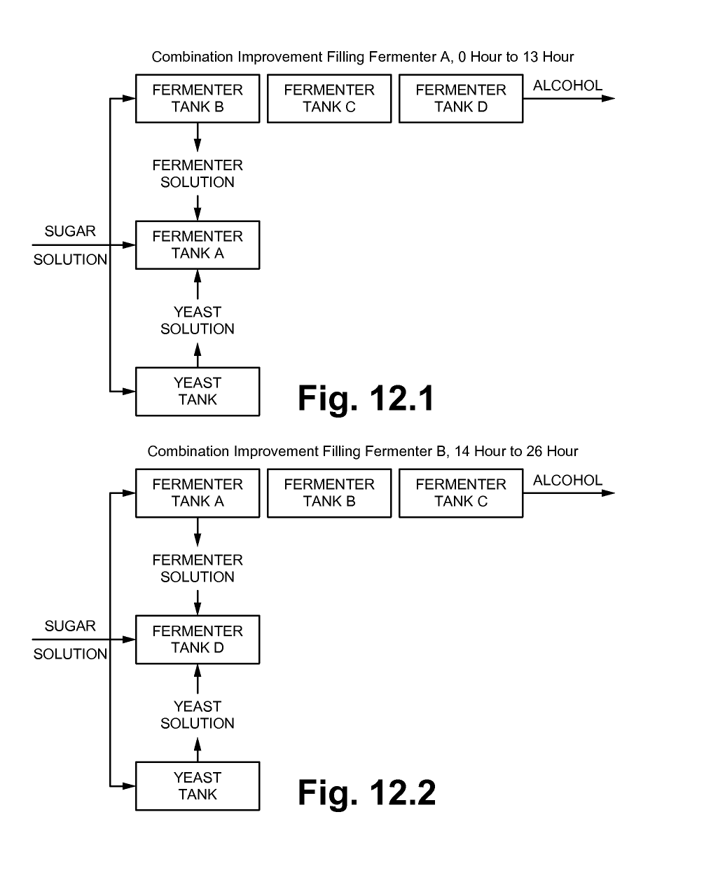

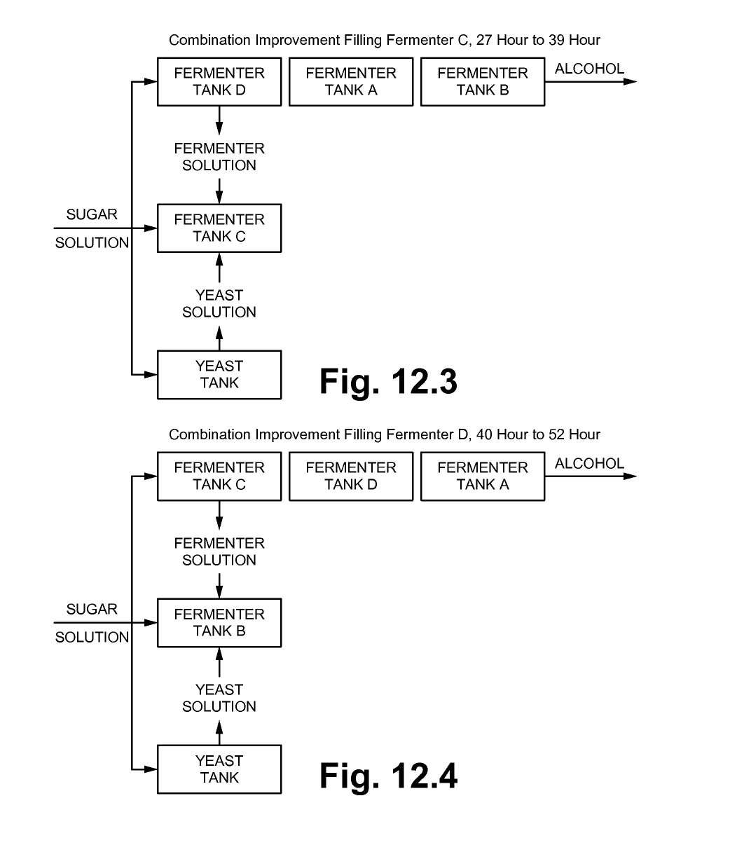

FIGS. 12.1 to 12.4 are flow diagrams of a Combination Improvement System for a 52 hour cycle in accordance with some embodiments of the present invention. The Yeast Tank Improvement System computer simulation and the Fermenter Tank Improvement System computer simulation have been shown to independently yield better overall results than the Current System. Therefore combining both new Systems together in a computer simulation would synergize the improvement efficiencies in both processes and yield even better overall results.

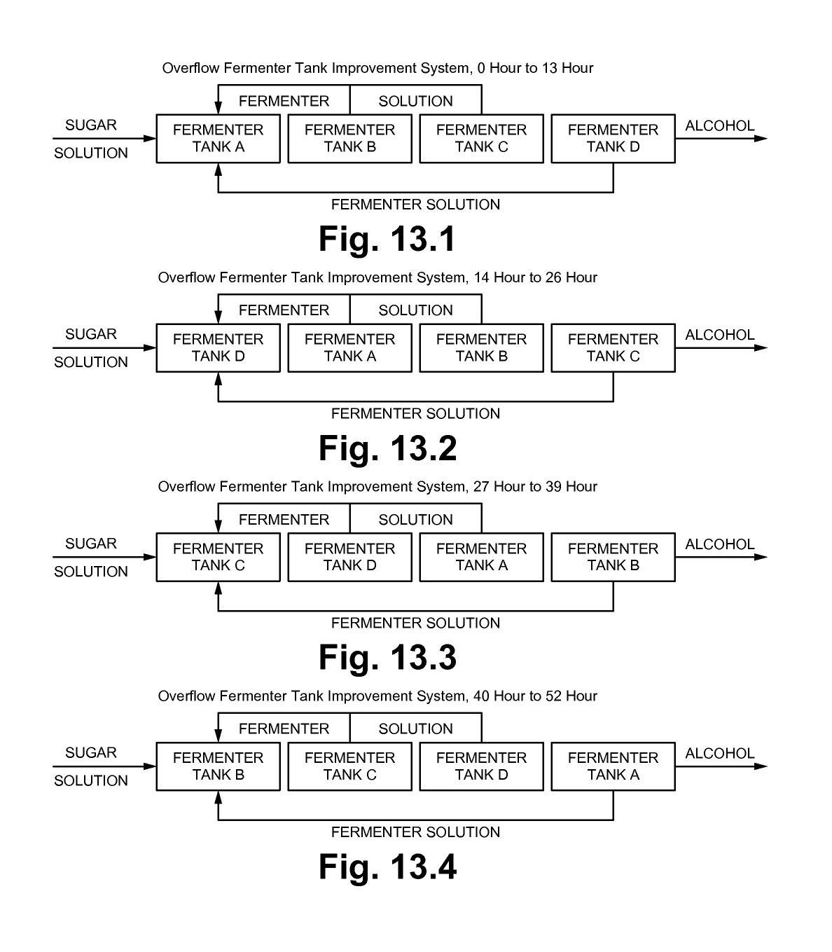

FIGS. 13.1 to 13.4 are flow diagrams of an Overflow Improvement Systems for a 52 hour cycle in accordance with some embodiments of the present invention. Fermenter Solution from Aged Fermenter Tanks at designated intervals. Taking the overflow froth layer from a downstream Aged Fermenter Tank and recycling the product back into a New Fermenter Tank will increase the overall yeast strength and rate change. This improved system can operate by itself or in conjunction with other improved systems (Low yeast tank improvement, high yeast tank improvement, fermenter tank improvement tank, or combination of yeast tank and fermenter tank improvement).

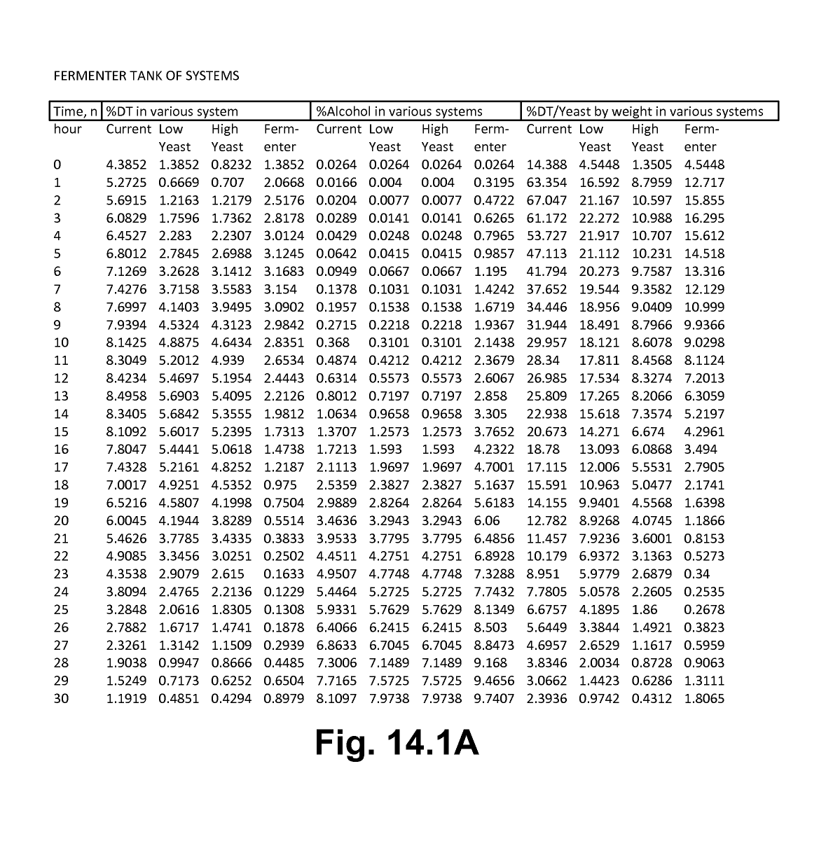

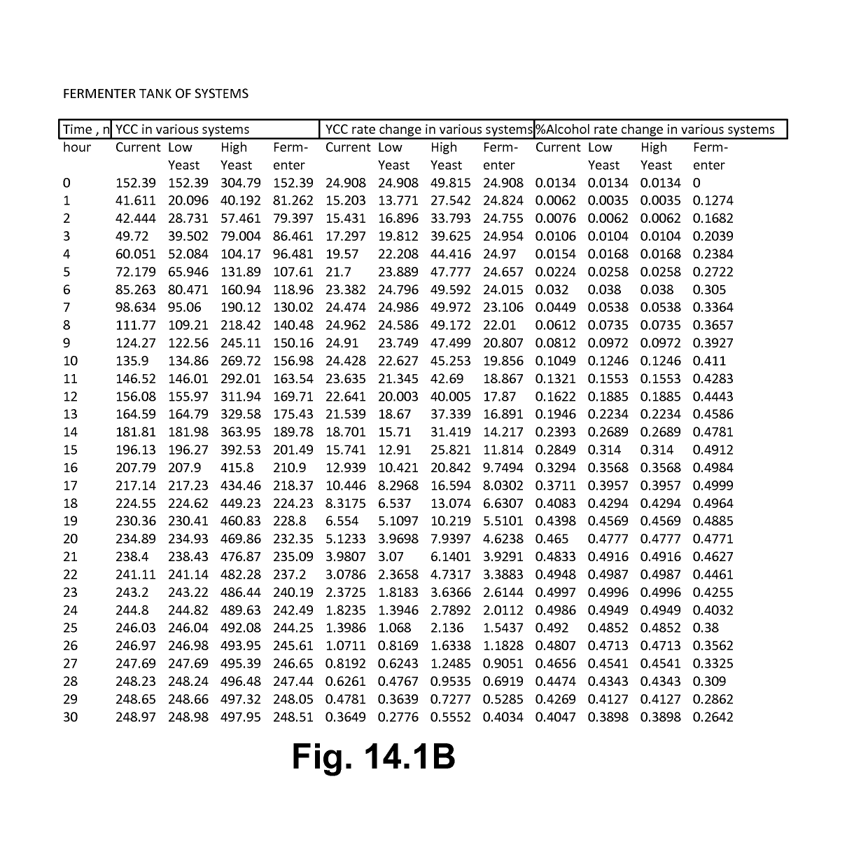

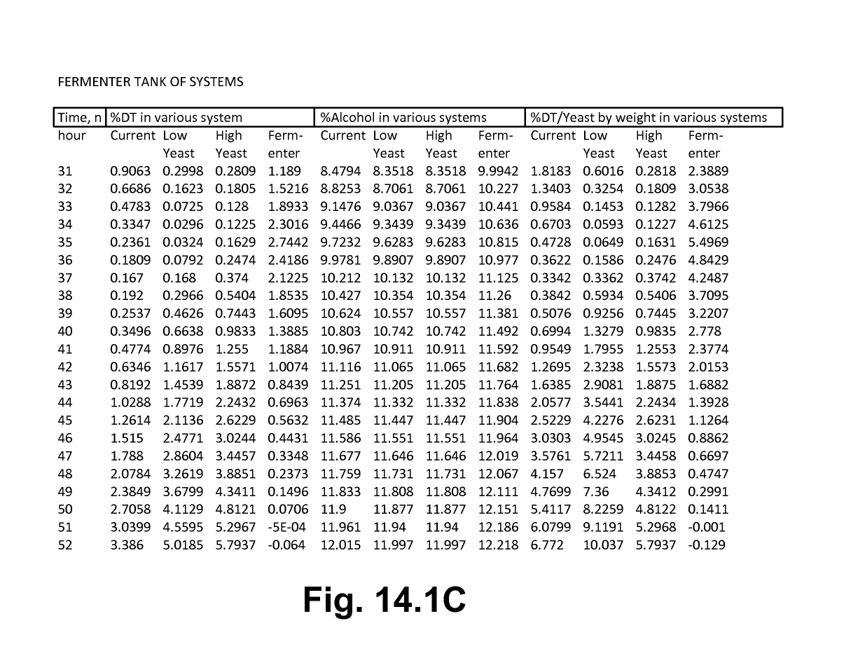

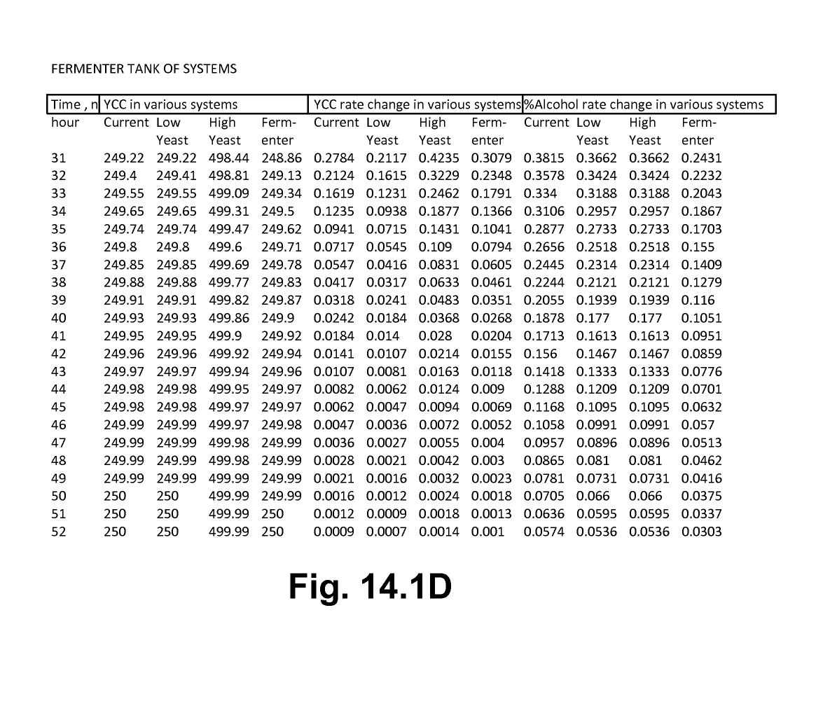

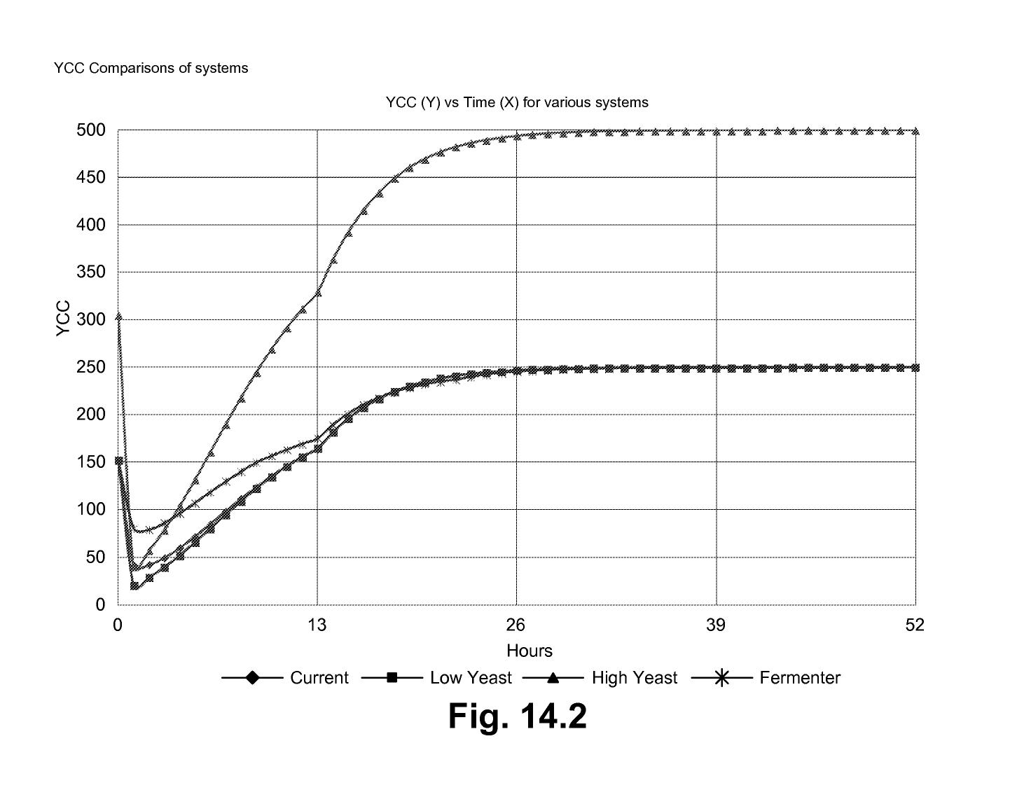

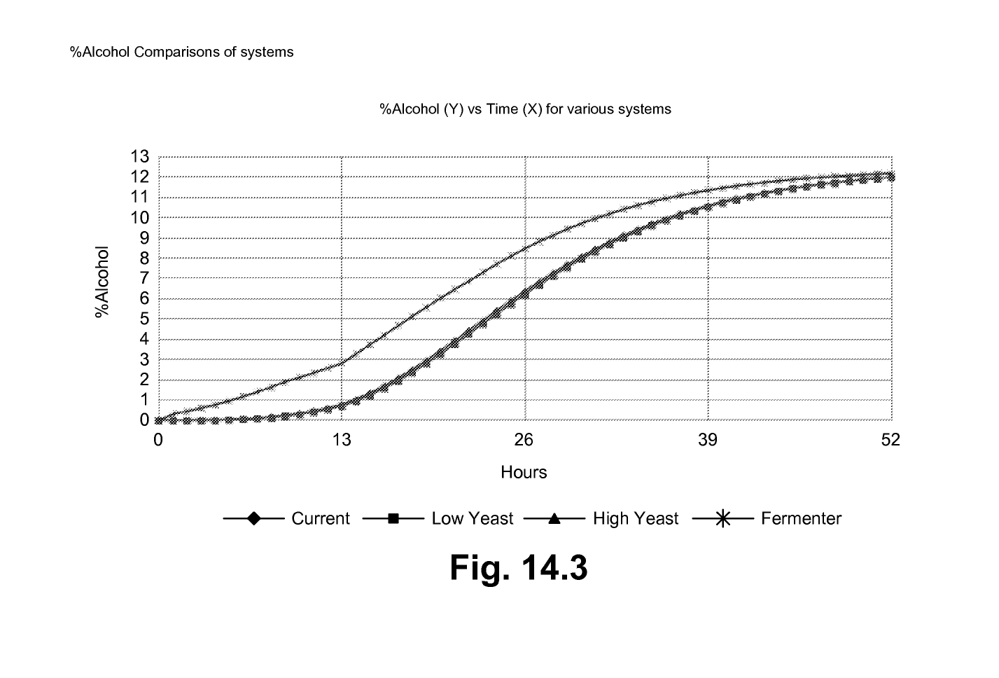

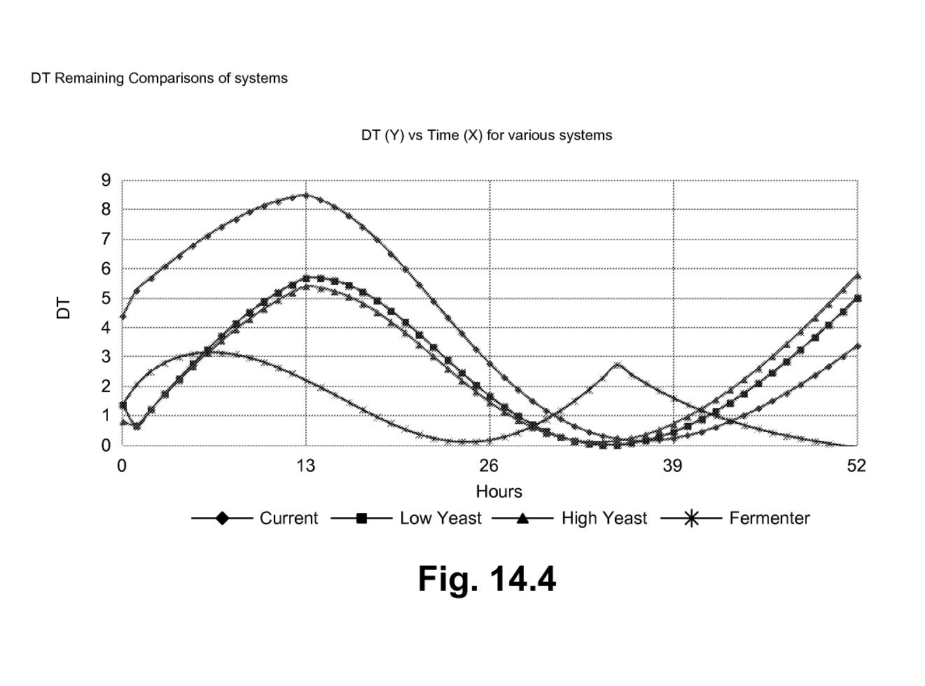

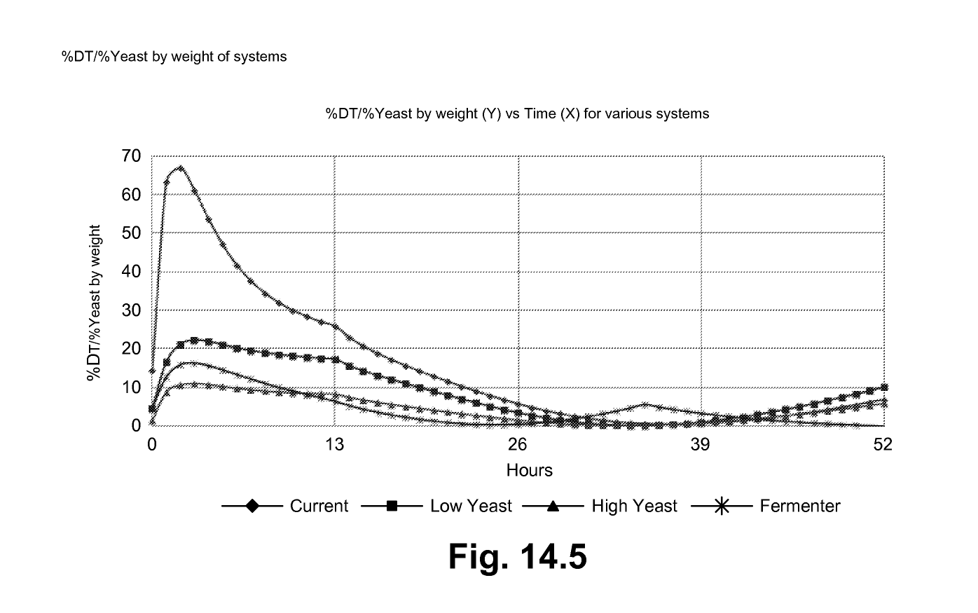

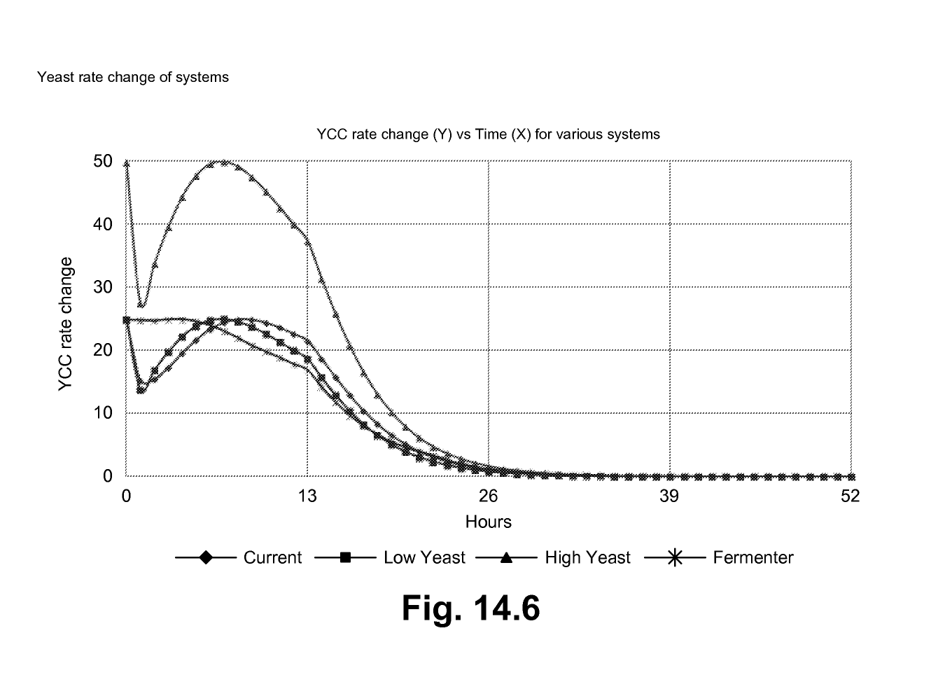

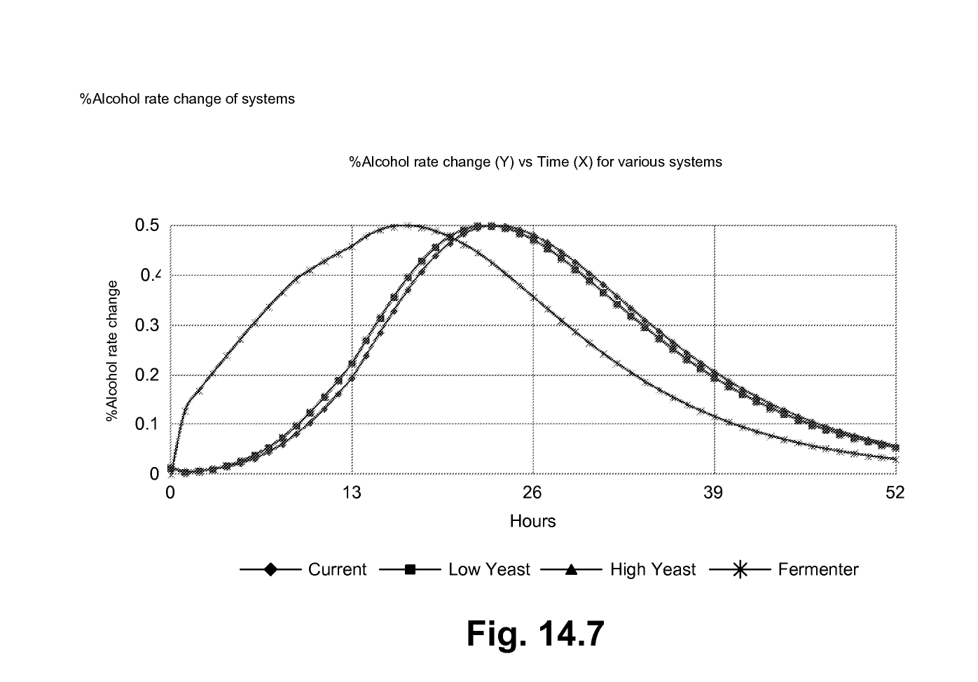

FIGS. 14.1A-14.1D comprise a table of all the Systems and their respective simulation data in accordance with some embodiments of the present invention. The data was taken from FIGS. 3.2A-3.2D, 8.3A-83.D, 9.3A-9.3D, and 11.2A-11.2D.

FIG. 14.2 is a plot of the YCC (Y) vs Time (X) of the Fermenter Tank of all the Systems in accordance with some embodiments of the present invention. The typical system starts with the same YCC as the Low Fermenter Tank Improvement System and Fermenter Tank Improvement System. After the initial drop in YCC, the Low Fermenter Tank Improvement catches up with the Current System by hour 13. This indicates that the yeast is growing faster and stronger in the Low Fermenter Tank Improvement System. The Fermenter Tank Improvement System outpaces the Current System until hour 17. This indicates that the yeast that has grown is strong and healthy and as a consequence will likely convert more starch into alcohol. The Current System also requires constantly growing yeast through purchased yeast addition for each freshly made Yeast Tank batch. When the Yeast Tank Improvement Systems or Fermenter Tank Improvement System reach steady state conditions, no additional purchased yeast will be required for each batch. This cost savings on purchased yeast could be as high as $200,000 a year and the total cost savings on reduced enzyme dosage could be as high as $300,000 a year.

FIG. 14.3 is a plot of % Alcohol (Y) vs Time (X) of the Fermenter Tank for all the Systems in accordance with some embodiments of the present invention. Fermenter Tank Improvement System produces more alcohol immediately with 0.32% at hour 1 and consistently outpaces the Current System. The Current System computer simulation yields 12.02% Alcohol. The Low Yeast Improvement System and High Yeast Improvement System computer simulations both yield 12.00% Alcohol. This is a minimal difference by data run off in computer simulation. The Fermenter Tank Improvement System, however, yields 12.22% Alcohol surpassing the output of the Current System which is an increase Alcohol yield of 1.64% [(12.22-12.02)/12.02*100]. In addition, the alcohol yields in each improved system are expected to be more than 2% increase based on typical yield of unwanted Glycerine in the Current System. If the net difference in Glycerine production is 0.5% (1.3-0.8) and assuming Glycerine to alcohol production ratio is 2 (two mass units of glycerol can be produced or one mass unit of ethanol from the same amount of sugar), then each improved system is expected to have an increase of 2% [0.5/(2*12*100)] alcohol yield by the decrease in Glycerine.

FIG. 14.4 is a plot of DT (Y) vs Time (X) of the Fermenter Tank for all the Systems in accordance with some embodiments of the present invention. All the improved systems have consistently lower amounts of DT compared to the Current System resulting in less osmotic stress to yeast. In order for alcohol to produce effectively during the hour 15 to hour 25, the DT should be 3% or less. The % DT in the Current System of this fermentation window ranges from 3.28 to 8.11 while the improved systems of the same fermentation window range from 0.13 to 5.69.

FIG. 14.5 is a plot of % DT/% Yeast by weight (Y) vs Time (X) of the Fermenter Tank for all the Systems in accordance with some embodiments of the present invention. All the improved systems have consistently lowers values of % DT/% Yeast by weight compared to the Current System. The higher values of % DT/% Yeast by weight in the Current System indicate there is too much food compared to yeast which will cause the yeast to become stressed.

FIG. 14.6 is a plot of YCC rate change (Y) vs Time (X) of the Fermenter Tank for all the Systems in accordance with some embodiments of the present invention. All the improved systems have the maximum YCC rate change occur earlier during fermentation. The Low Yeast Tank Improvement System and High Yeast Tank Improvement System both reaches maximum YCC rate at hour at hour 7. The Fermentation Tank Improvement System has reaches a maximum YCC rate at hour of 1 to 4. The sooner into the fermentation cycle the maximum yeast rate change occurs, the more time the yeast has to convert sugar to alcohol. The faster sugar is converted to alcohol the lower the concentration of sugar in the solution. The lower the concentration of sugar in solution the more effective the saccharifying enzyme can work because of reduced end-product inhibition. It is a common occurrence in the industry to see that poor yeast performance results in incomplete saccharification enzyme conversion of dextrins and starch to sugar.

FIG. 14.7 is a plot of Alcohol rate change (Y) vs Time (X) of the Fermenter Tank for all the Systems in accordance with some embodiments of the present invention. The Fermenter Tank improvement has the largest Alcohol rate change in Fermenter Tank Improvement begins at 0.12 at hour 0 and continue increase to 0.5 at hour 16 whereas the other systems have no or very little Alcohol rate change at hour of 0 to 7 then graduate increase to 0.5 at hour 23 to 25.

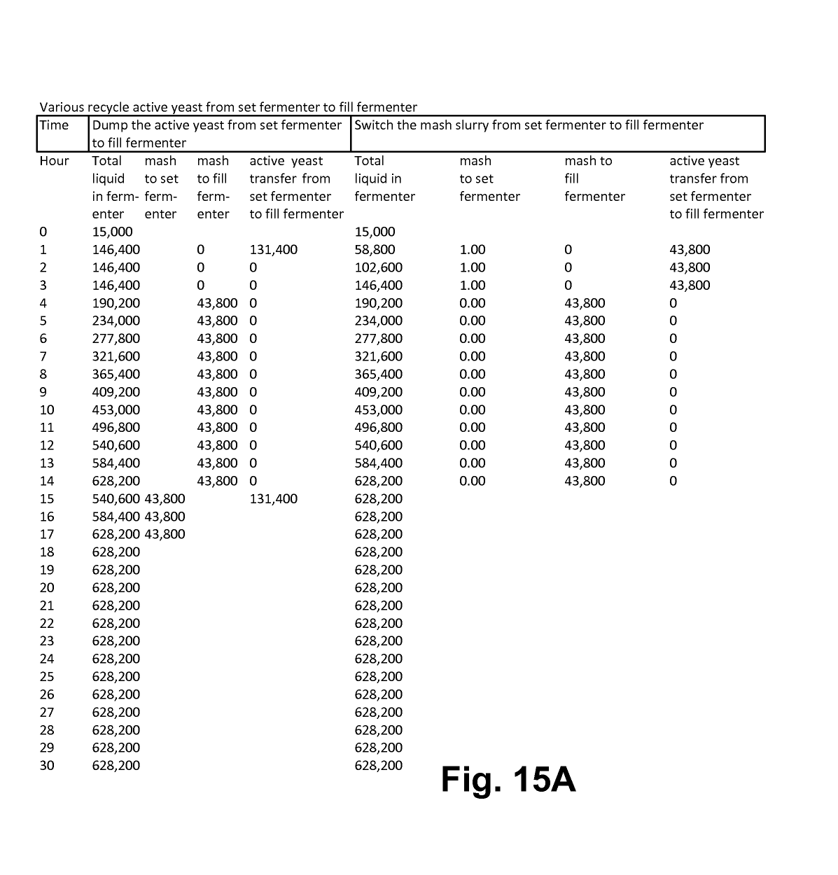

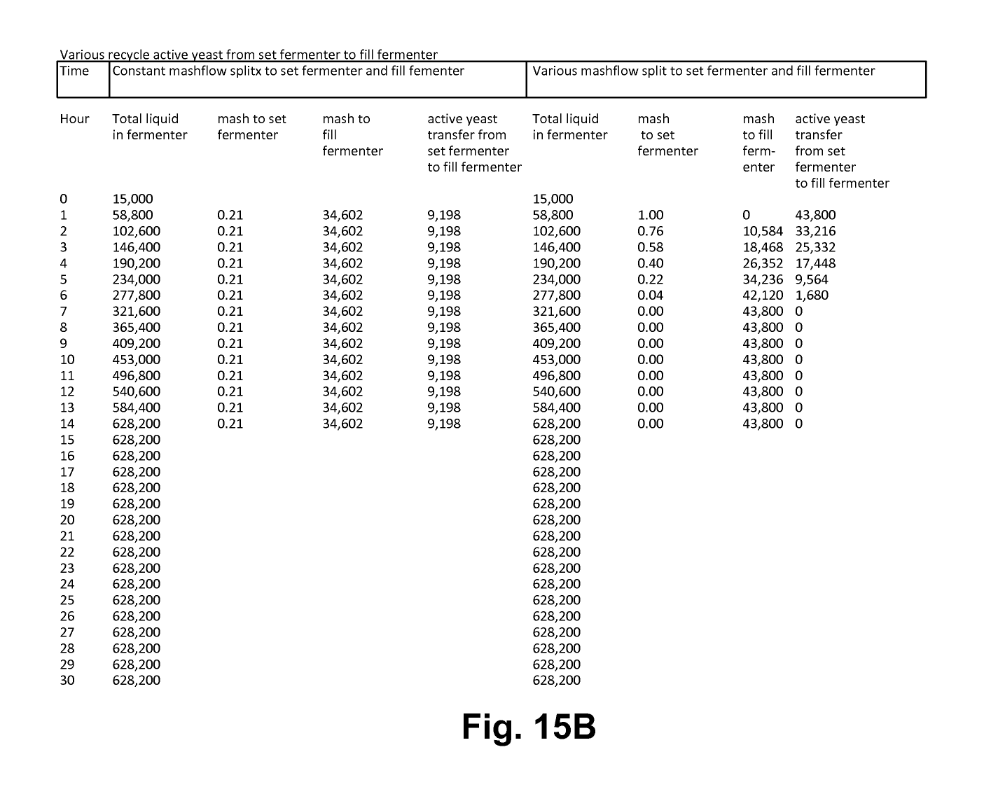

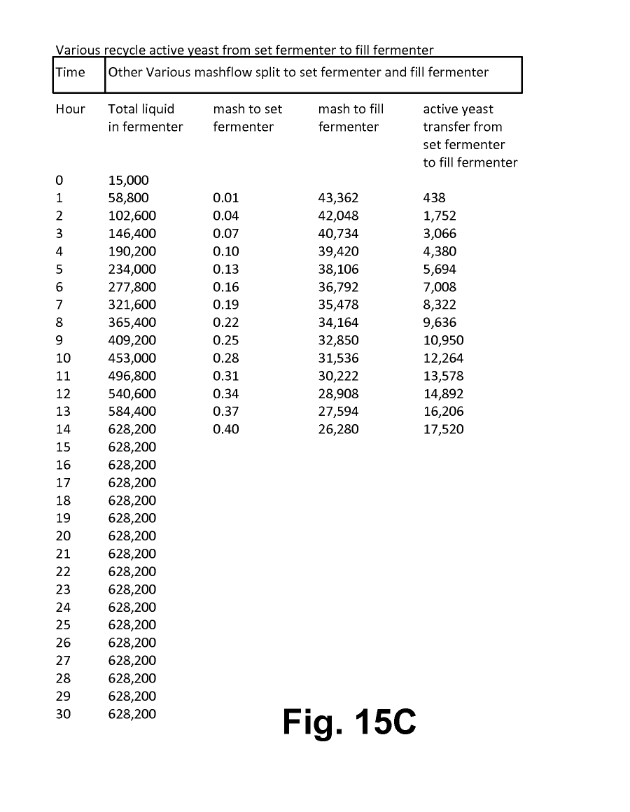

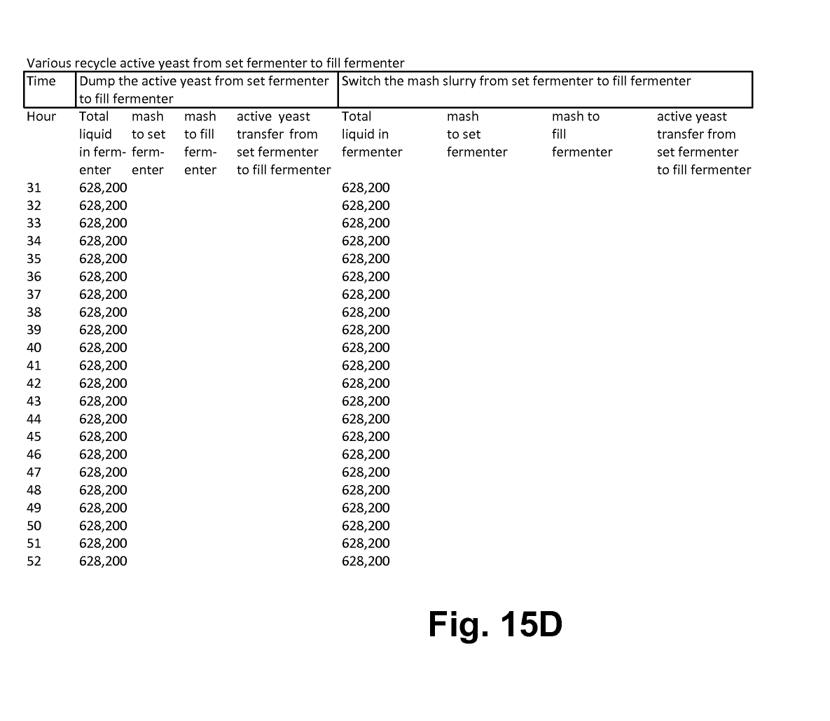

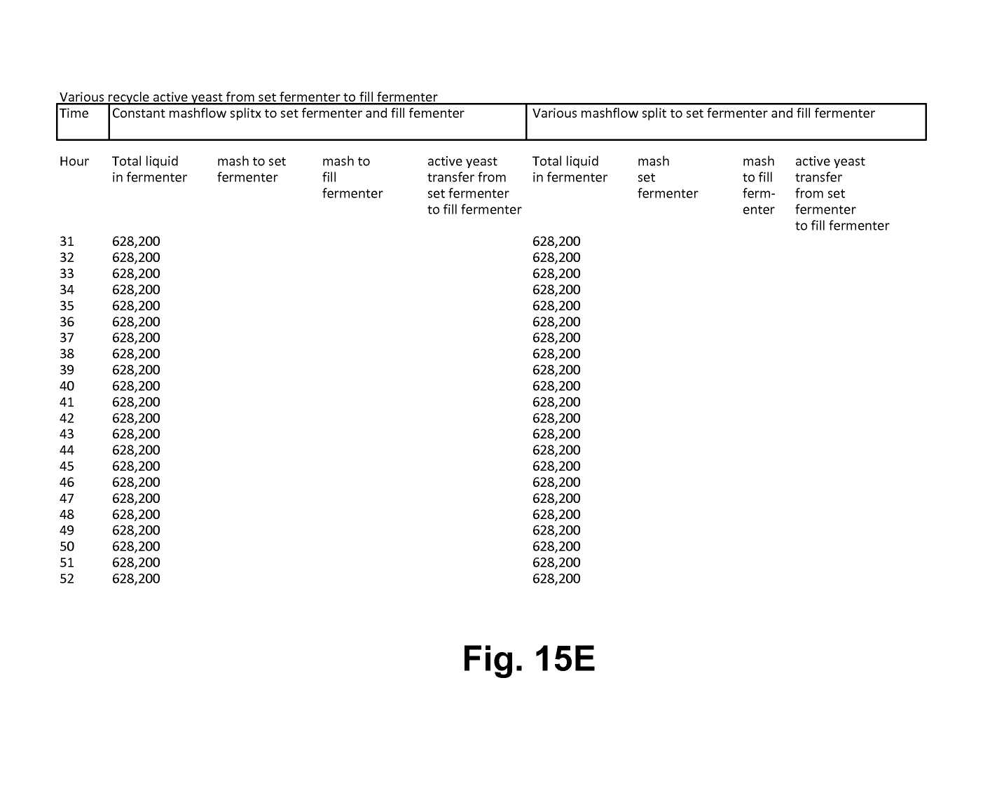

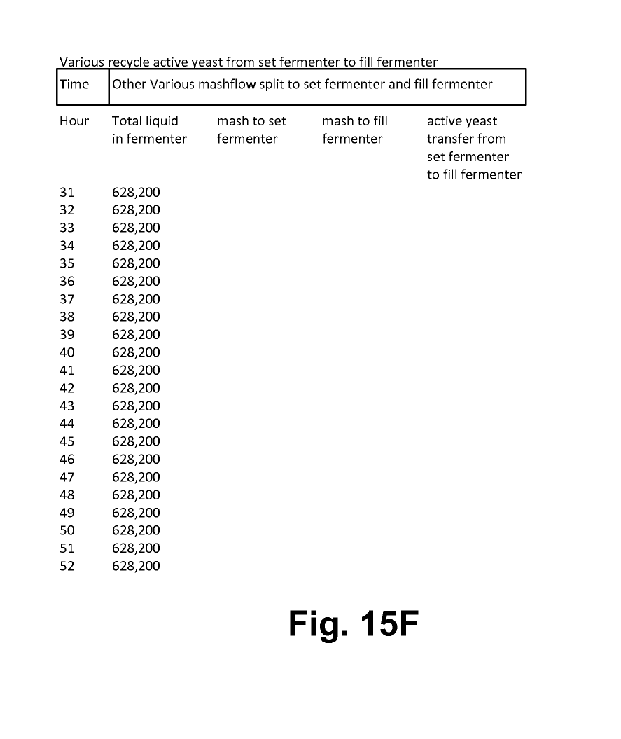

From the FIG. 14.7 plot, the % alcohol increases by recycling the most active yeast from the set fermenter to the fill fermenter. There are many way to recycle the most active yeast from the set fermenter to the fill fermenter. FIGS. 15A-15F show, of the five different recycle methods modeled, the 21% recycle produces the most active yeast set up in accordance with some embodiments of the present invention.

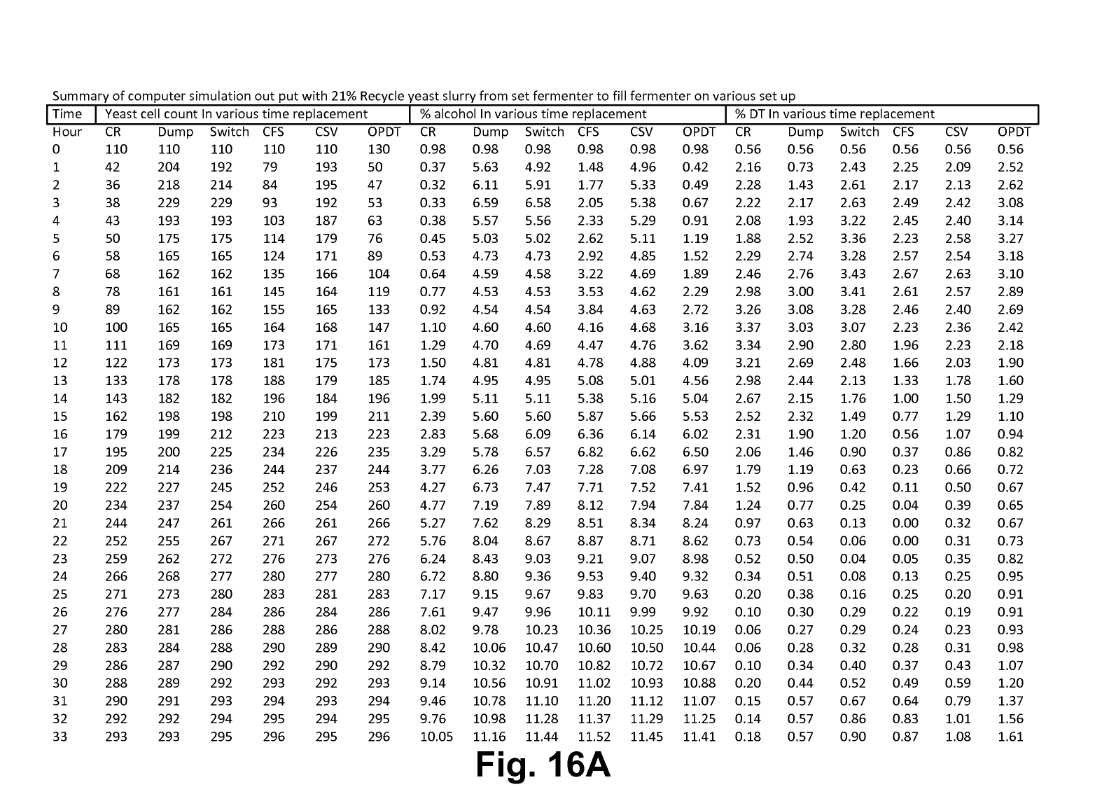

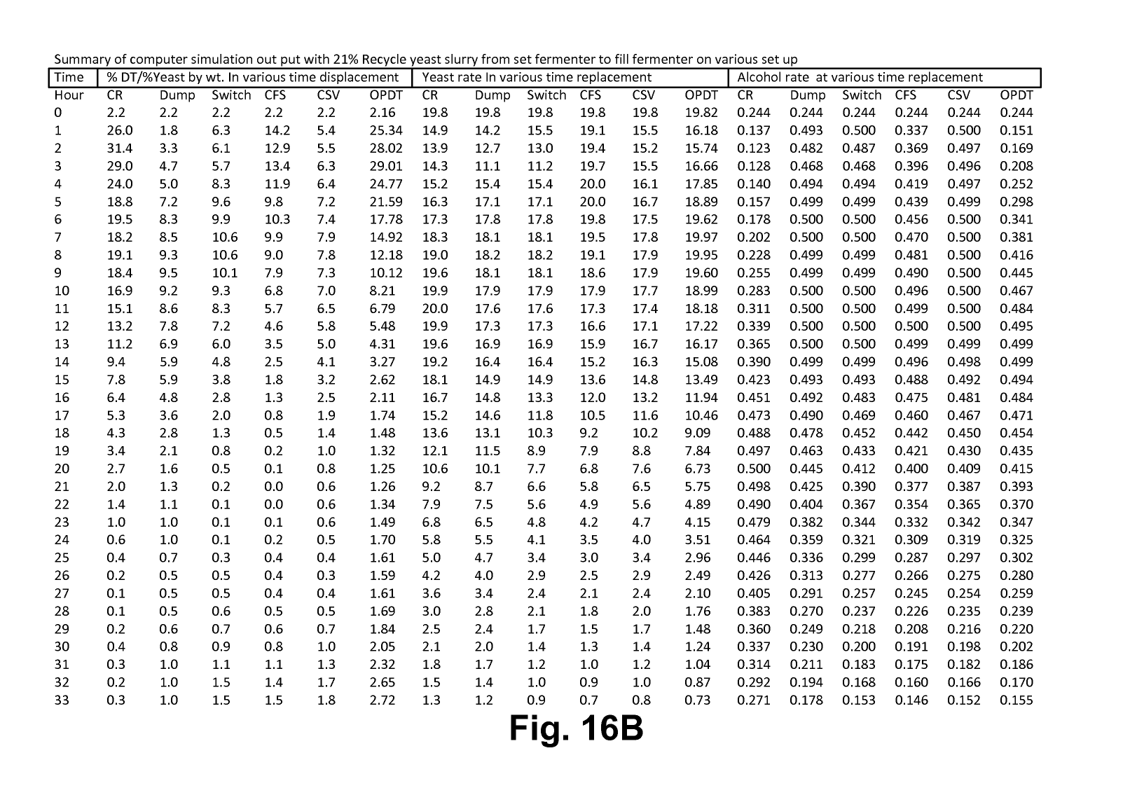

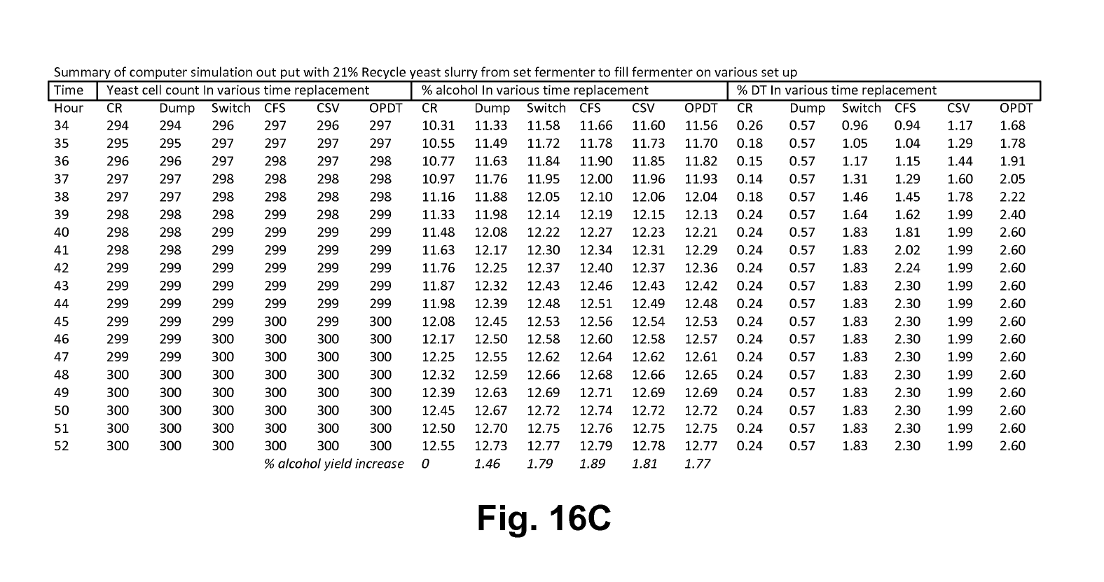

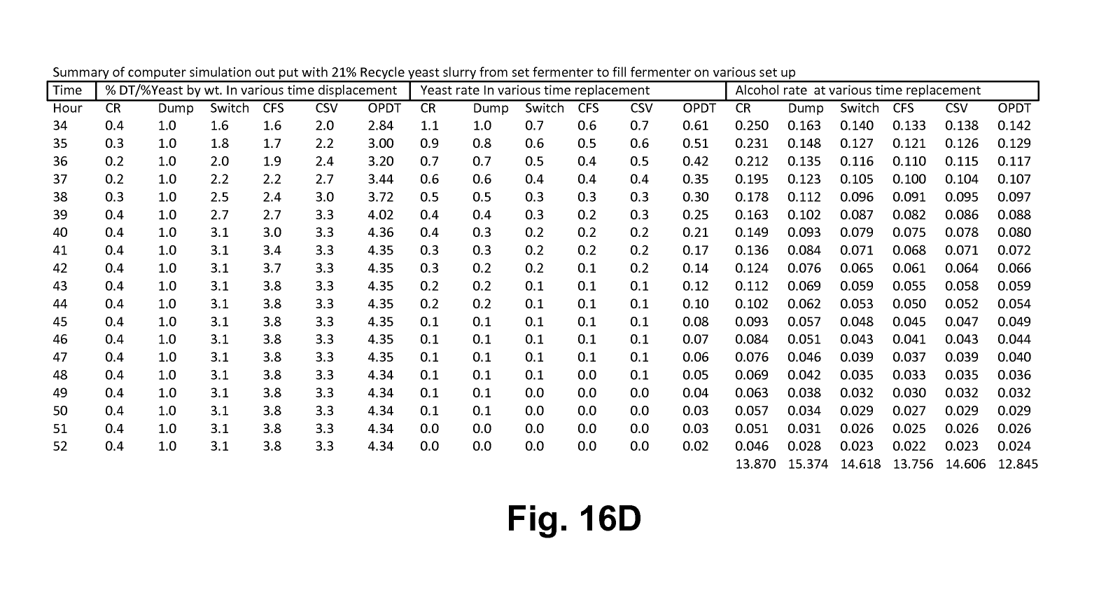

FIG. 16 summarizes the computer simulation output on the above five different recycle set up in accordance with some embodiments of the present invention. One of 60 MGY plant with 15,000 gal operation capacity of yeast tank, and four 628,000 gal operation capacity fermenter with 730 gal per minute of average mash rate with 14 hour of total fill time are used on those calculation.

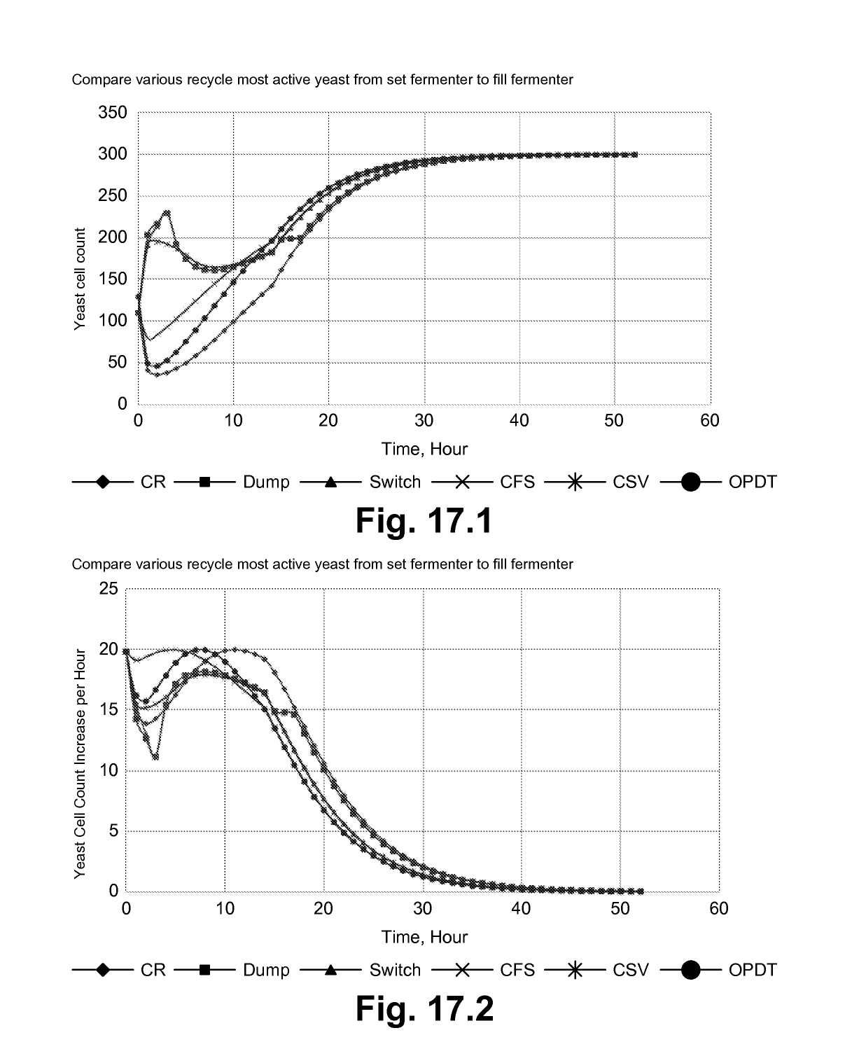

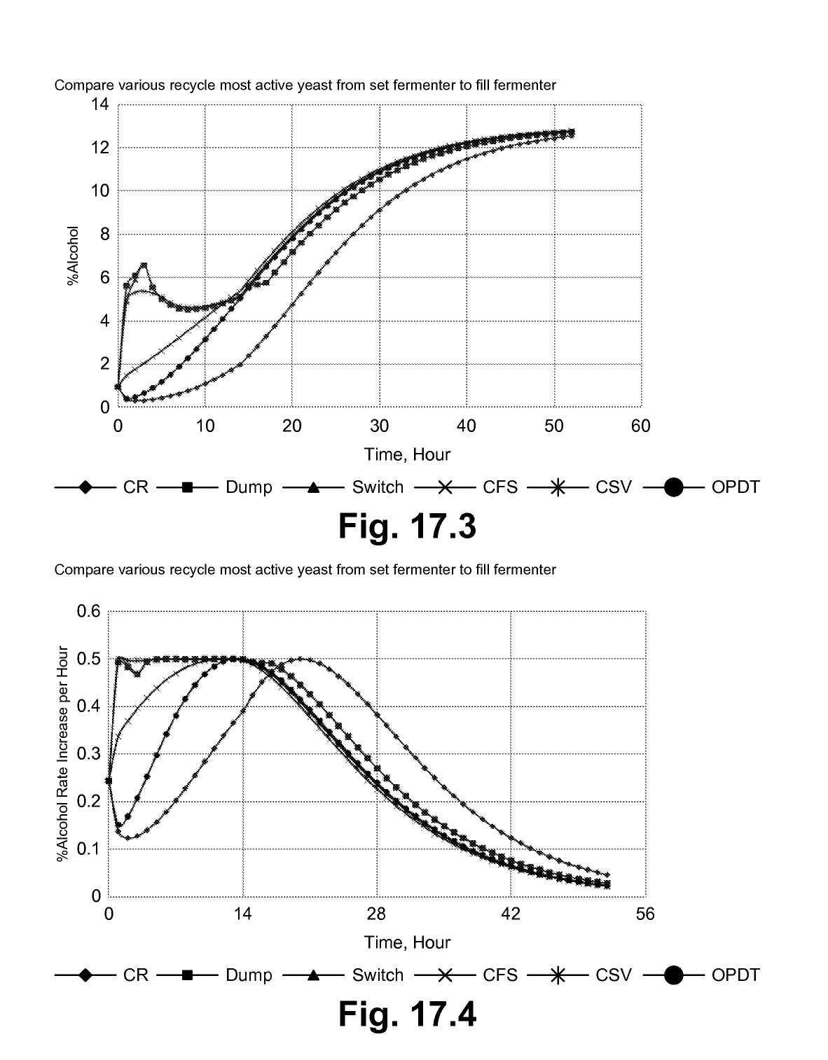

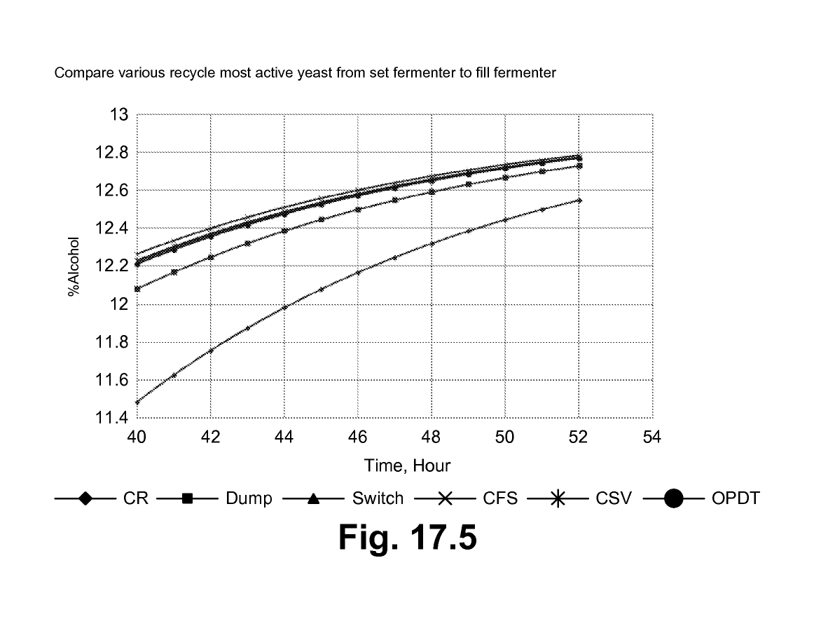

FIGS. 17.1-17.5 show the comparison data and plots in accordance with some embodiments of the present invention. The FIG. 17.1 is the yeast cell count vs time on various set up. FIG. 17.2 is yeast cell rate increase vs time on various set up. FIG. 17.3 is % alcohol VS Time on various set up. FIG. 17.4 is % alcohol rate increase VS time on various set up. FIG. 17.5 is an expanded view of FIG. 17.3 highlighting the end of fermentation and increased ethanol yield afforded by the invention.

By comparing all five different recycle most active yeast methods, it is clear that the constant flow split method gives the best result by comparing the % alcohol at drop.

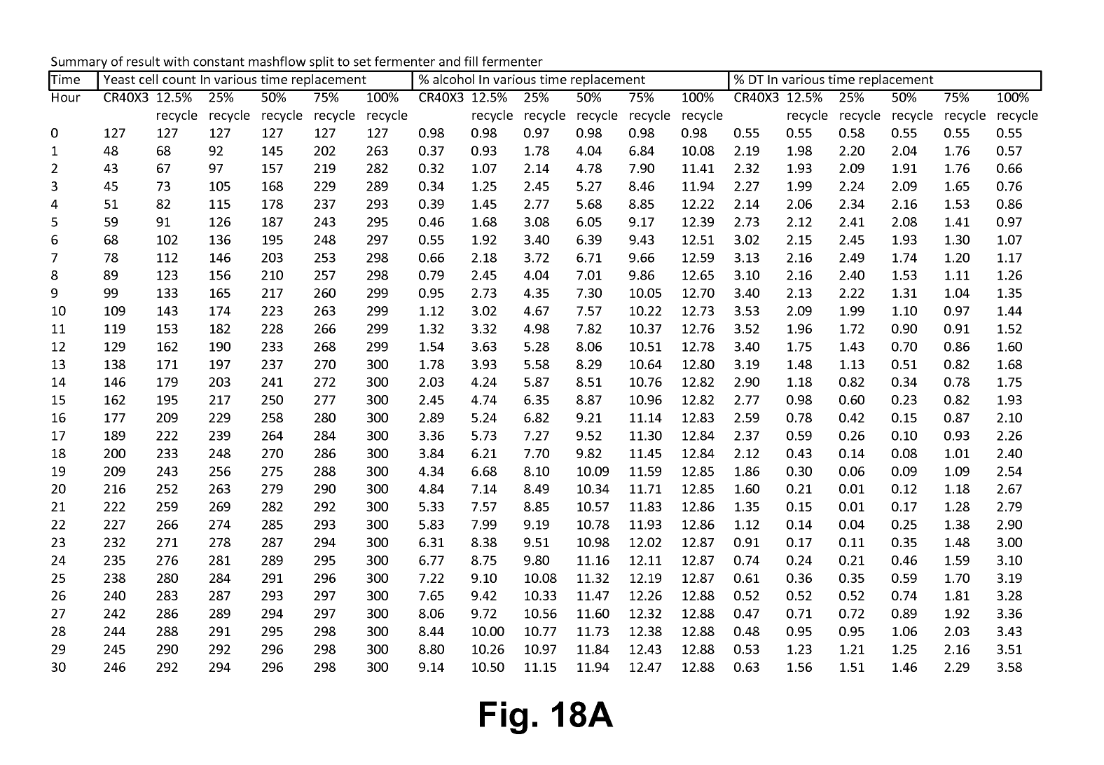

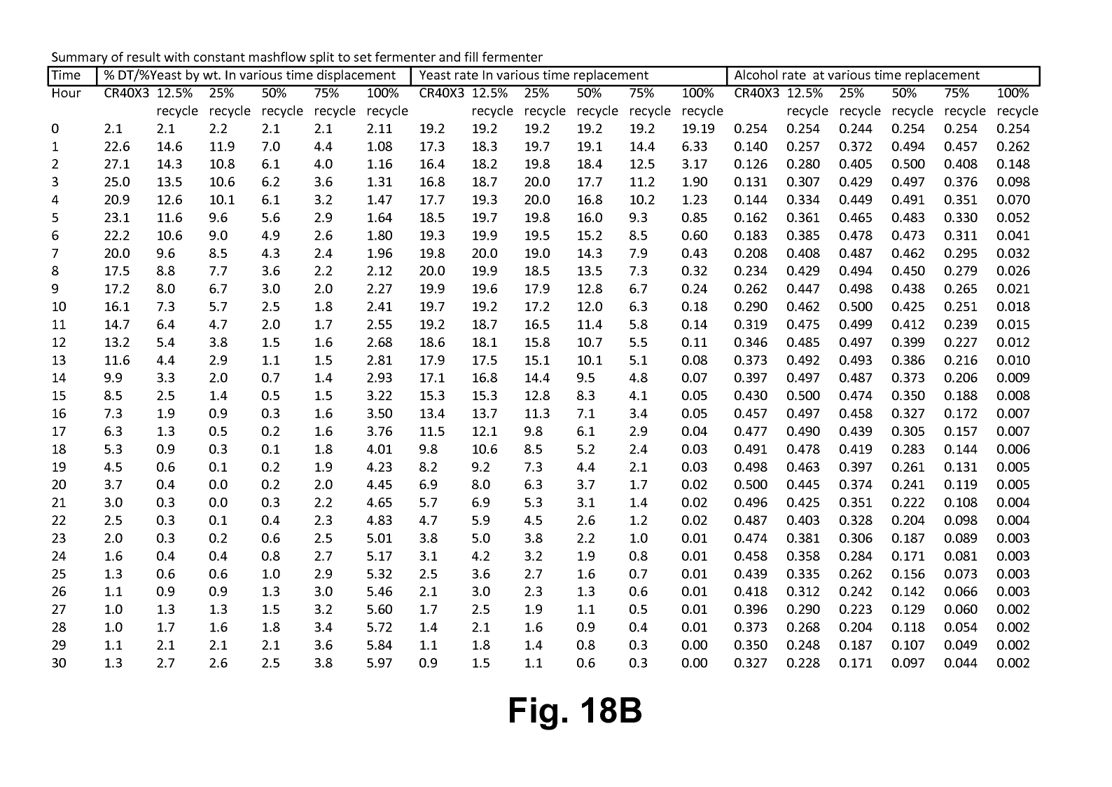

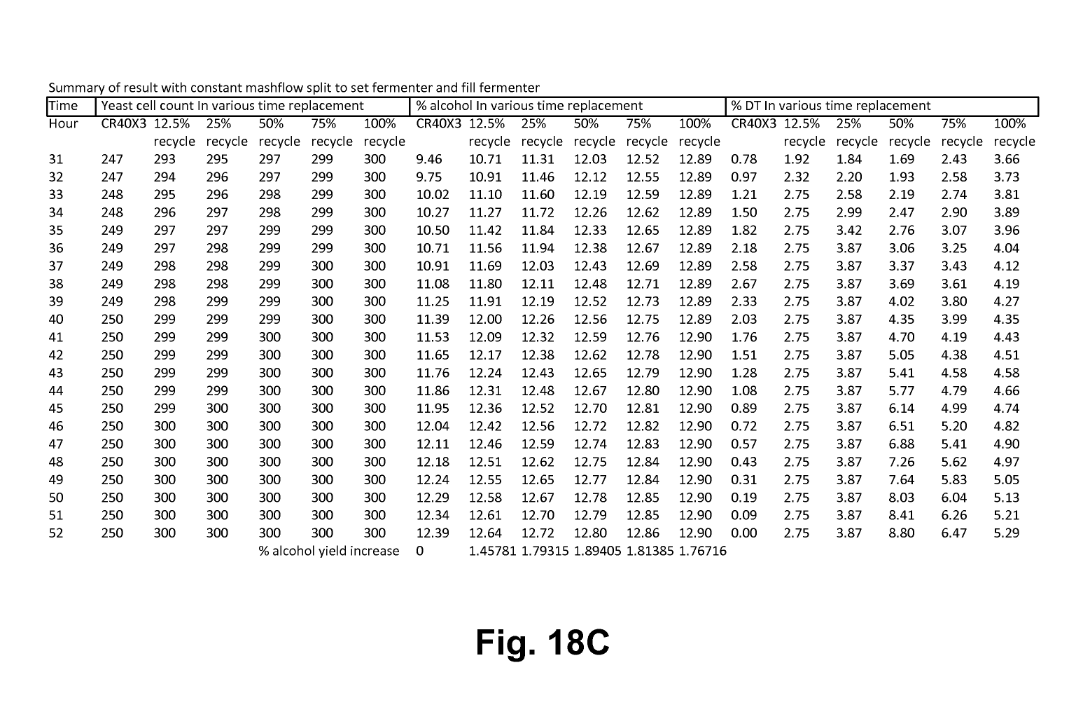

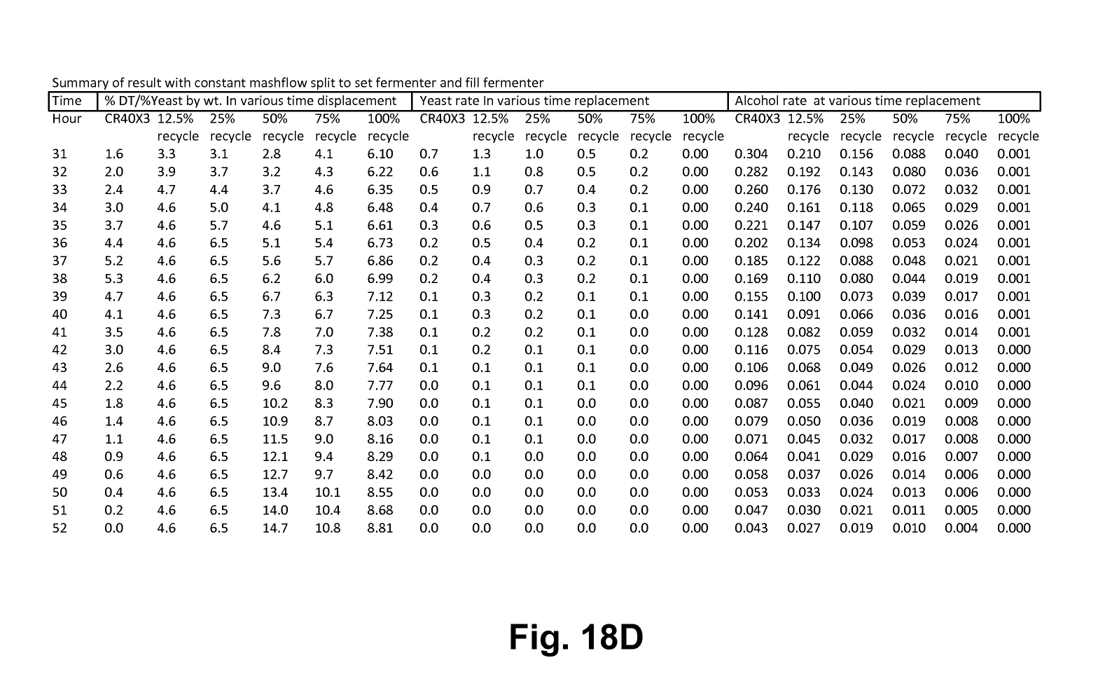

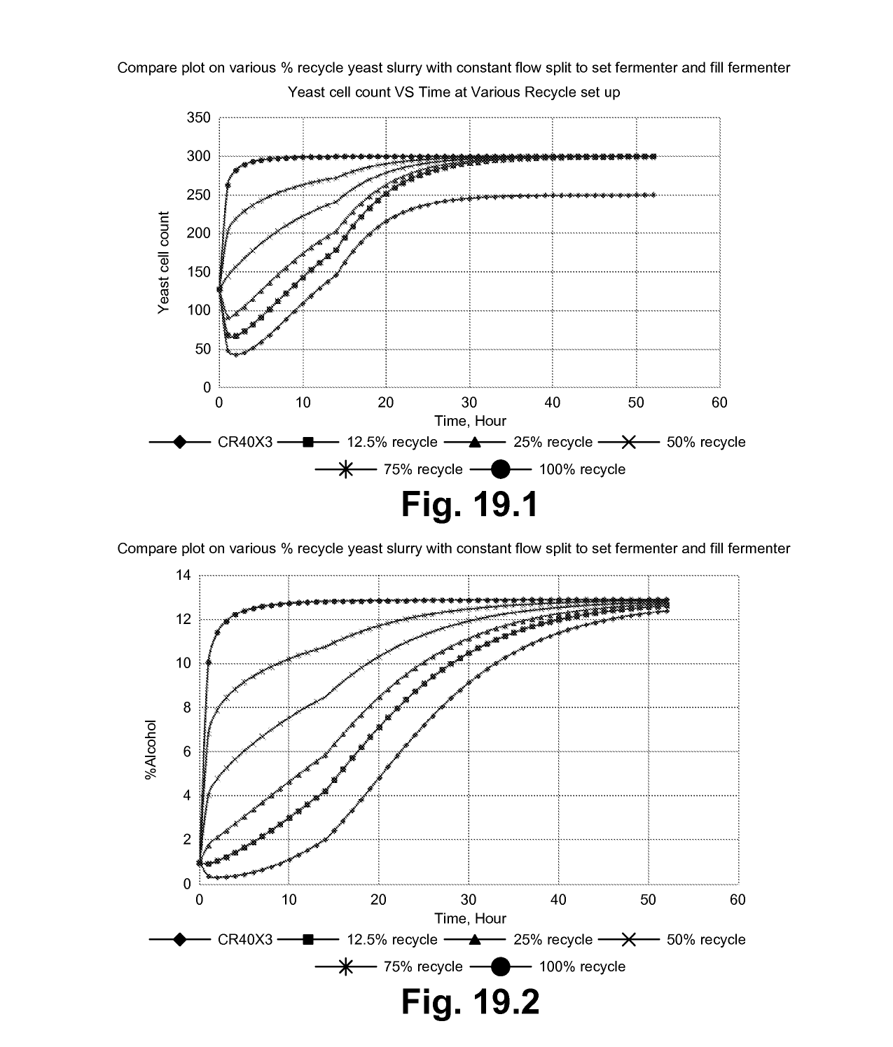

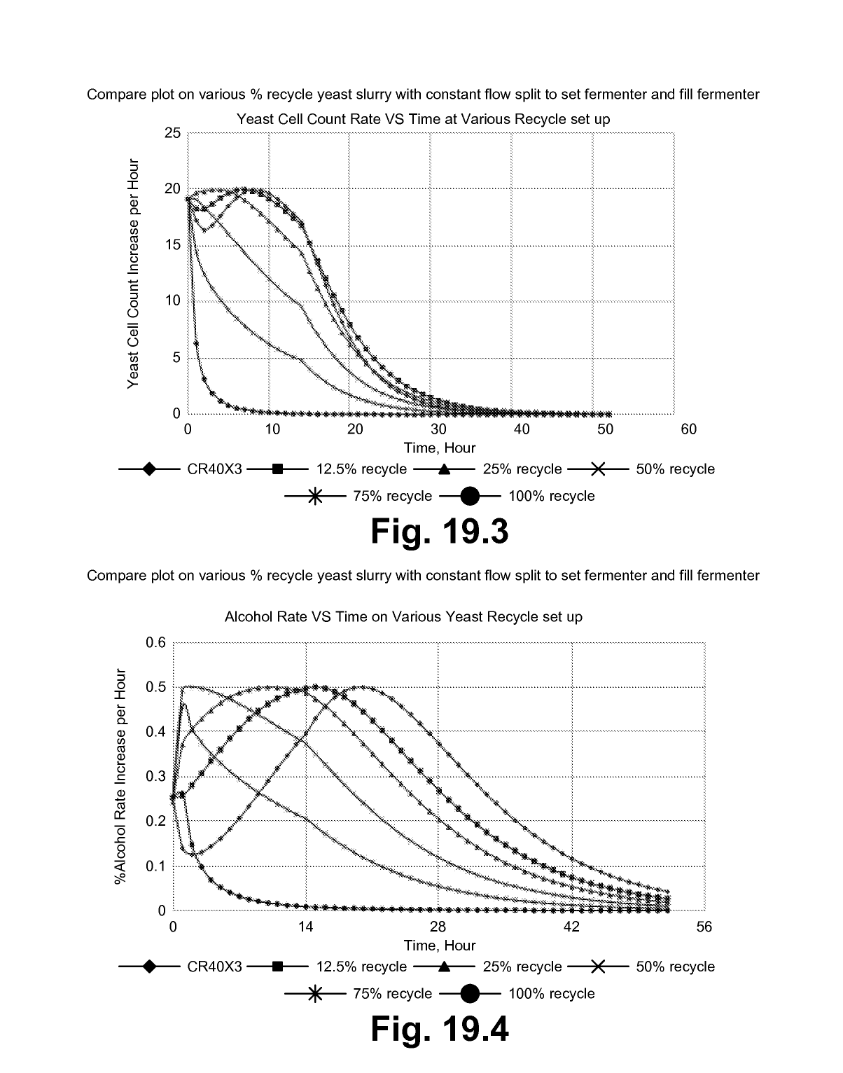

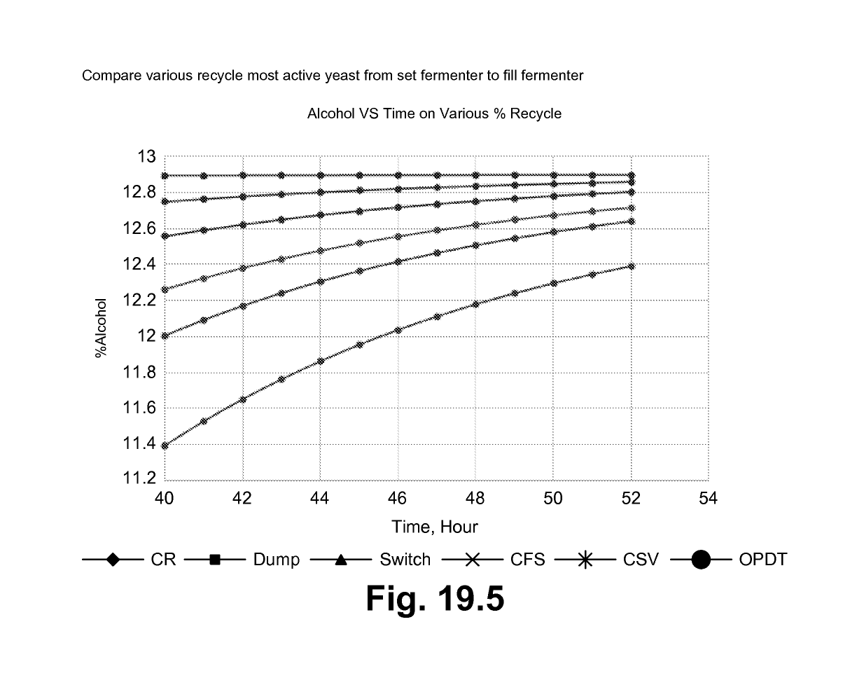

FIG. 18 shows the various % recycle on constant flow split set up are modeled on the simulation program in accordance with some embodiments of the present invention. The Summary of the computer simulation program output on various % recycle (12.5%, 25%, 50% 75% and 100%). The data are plotted on FIGS. 19.1-19.5. The FIG. 19.1 is Yeast count vs time on various % recycle. The FIG. 19.2 is Yeast cell count increase rate vs time on various % recycle. The FIG. 19.3 is the % alcohol VS time on various % recycle, and FIG. 19.4 is % alcohol rate increase vs time on various % recycle. FIG. 19.5 is an expanded view of FIG. 19.3 highlighting the end of fermentation and increased ethanol yield afforded by the invention.

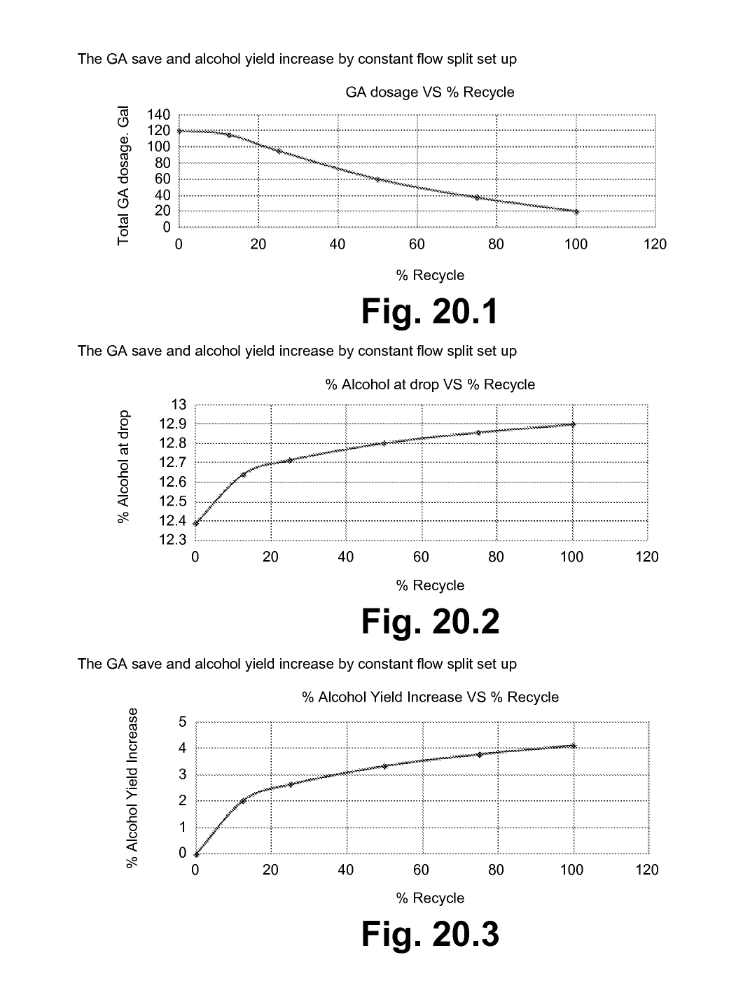

FIG. 20.2 shows that by compare % alcohol at drop on the various % recycle data, it can be observed that with only 12.5% recycle there is a big jump in % alcohol at drop (from 12.39 to 12.65%). This increase slows down with higher % recycle rates. FIG. 20.3 shows the % alcohol yield increase for various % recycle yeast rates. FIG. 20.1 shows the saccharifying Enzyme dosage used on various % recycle yeast simulations.

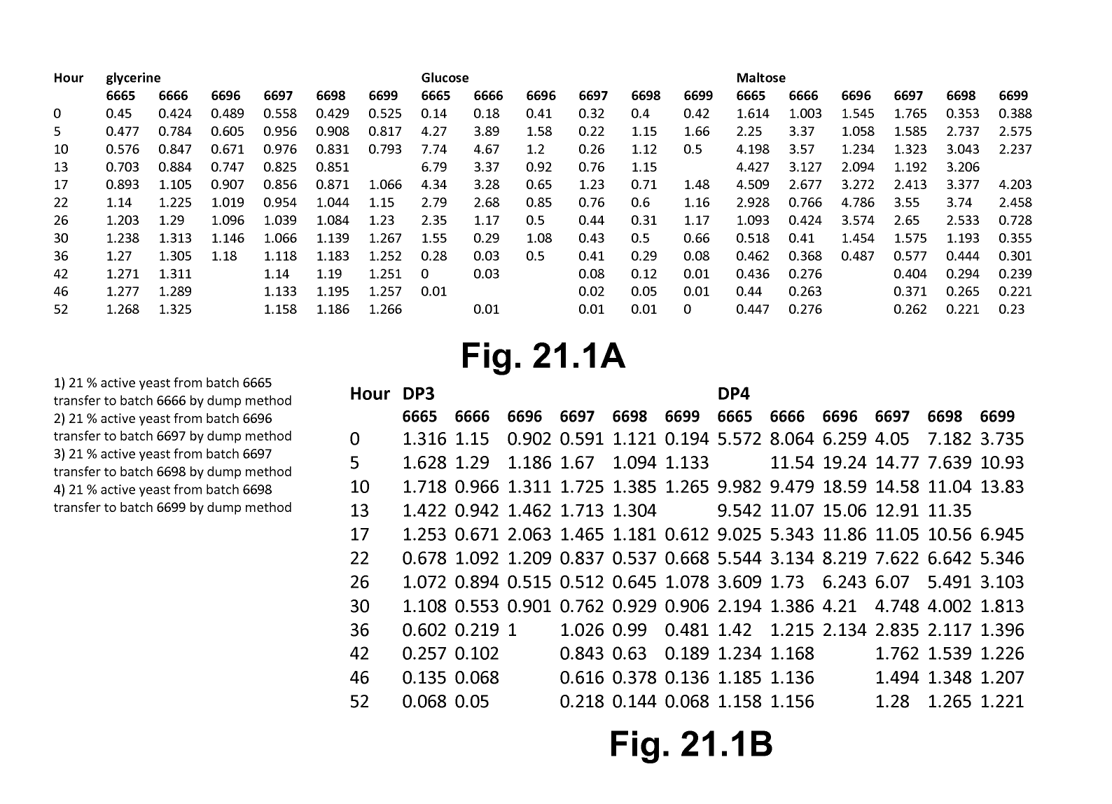

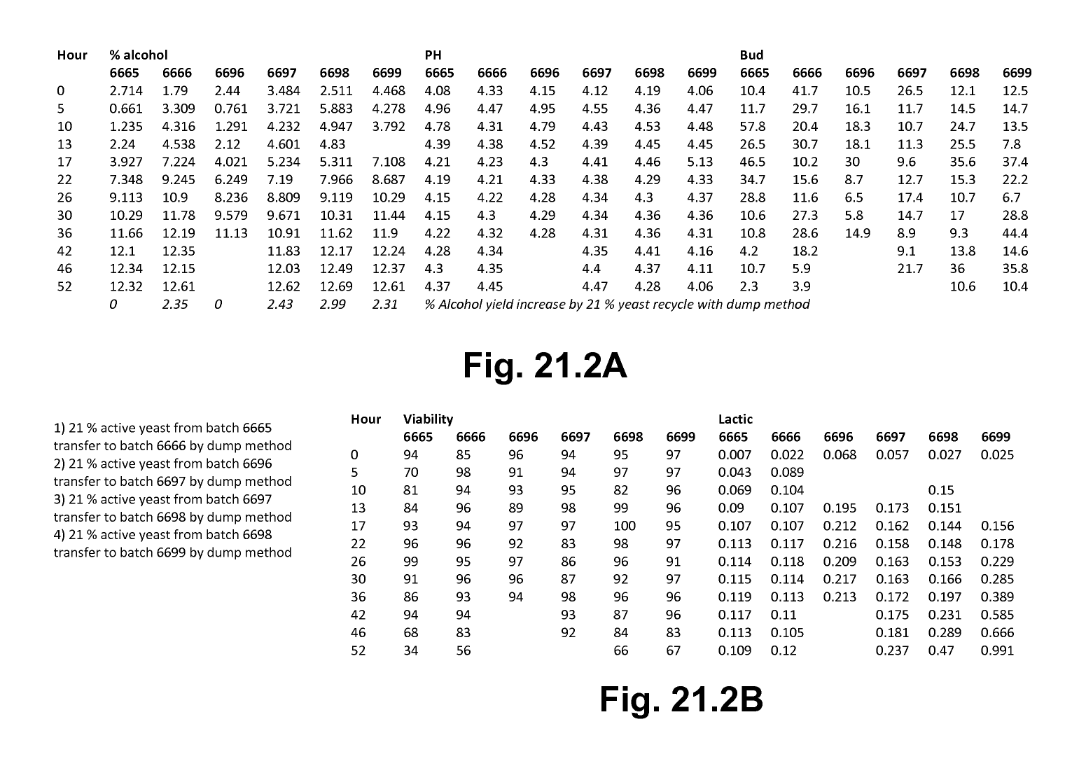

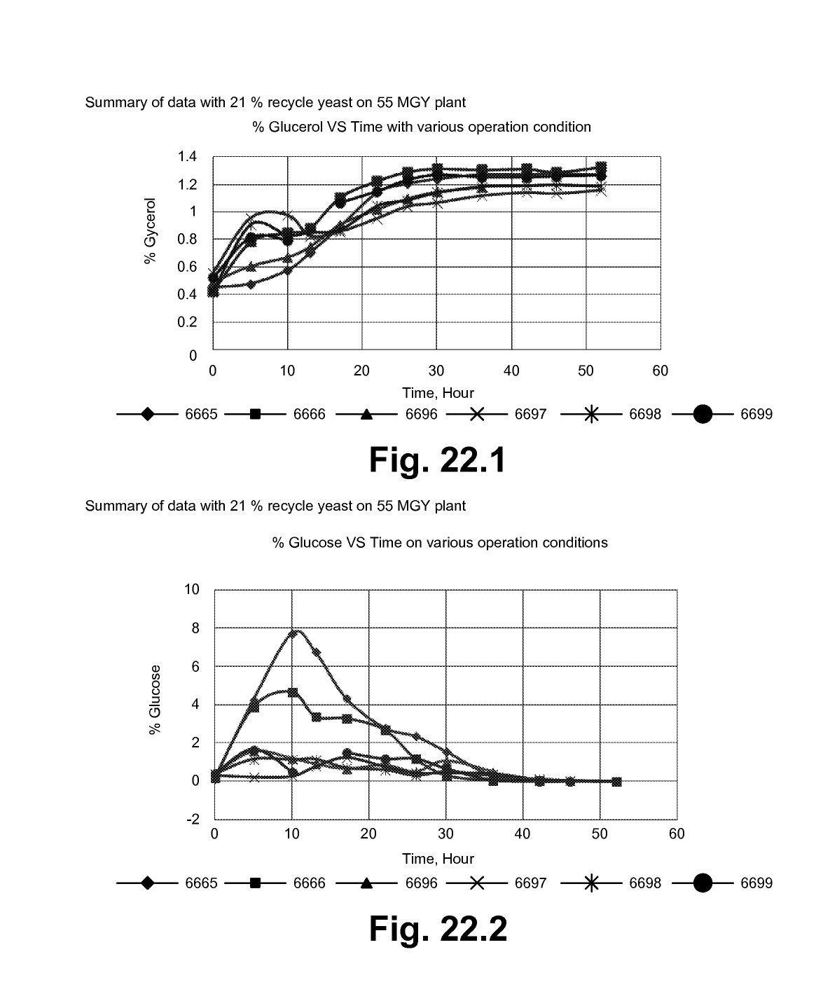

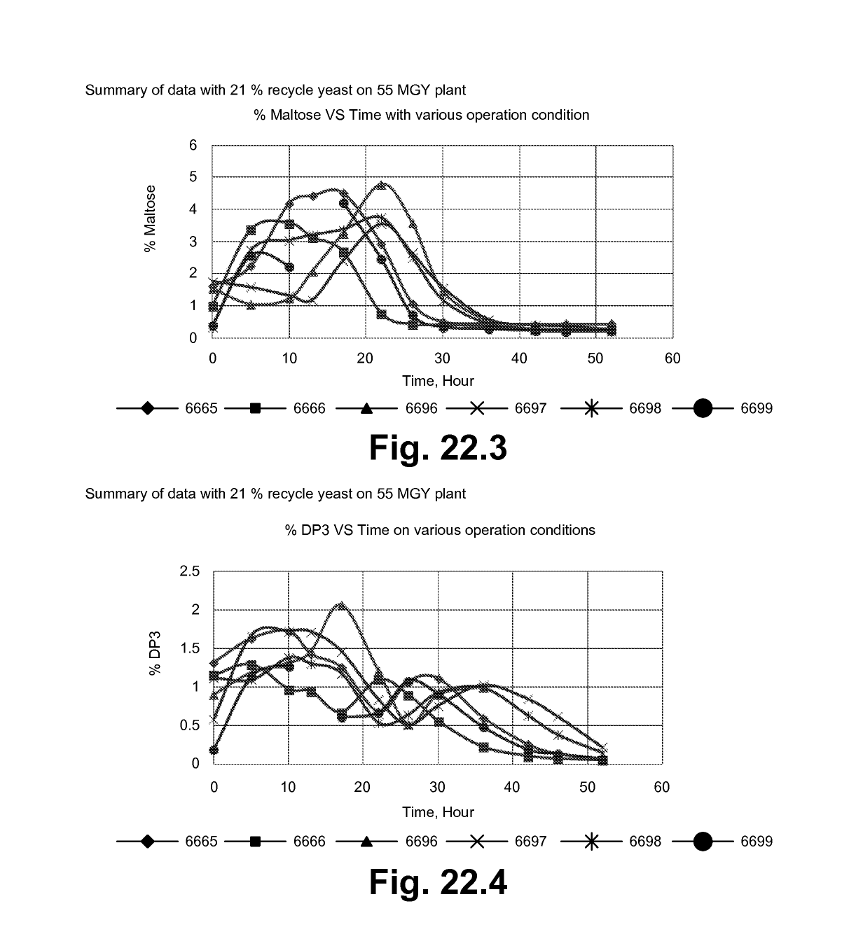

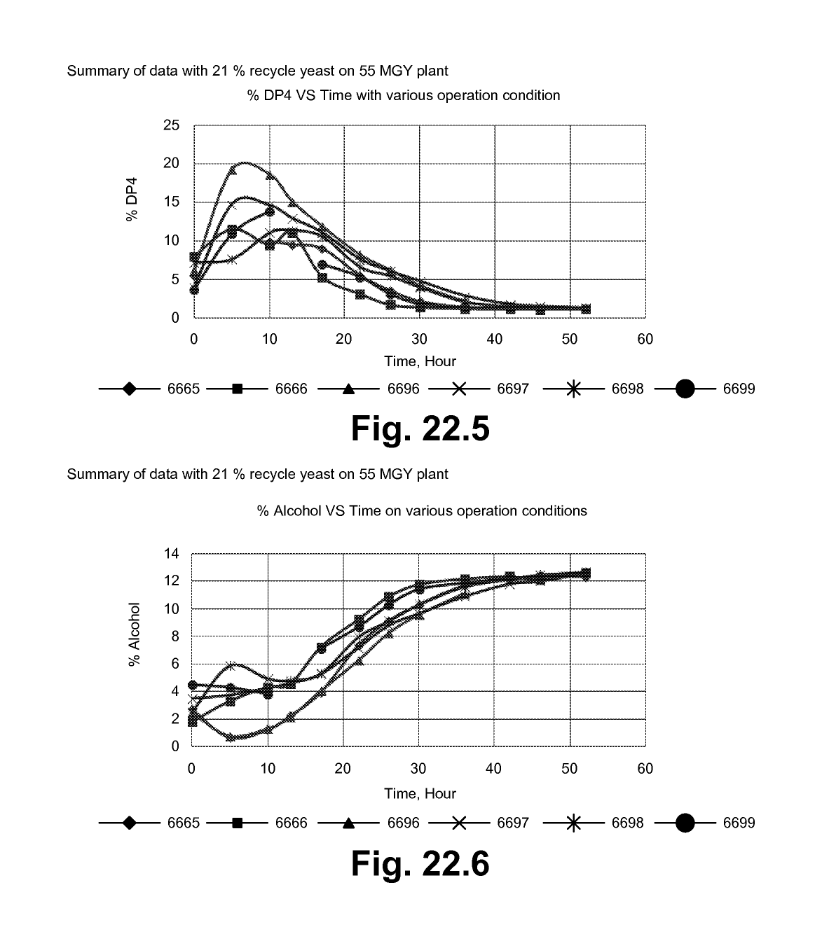

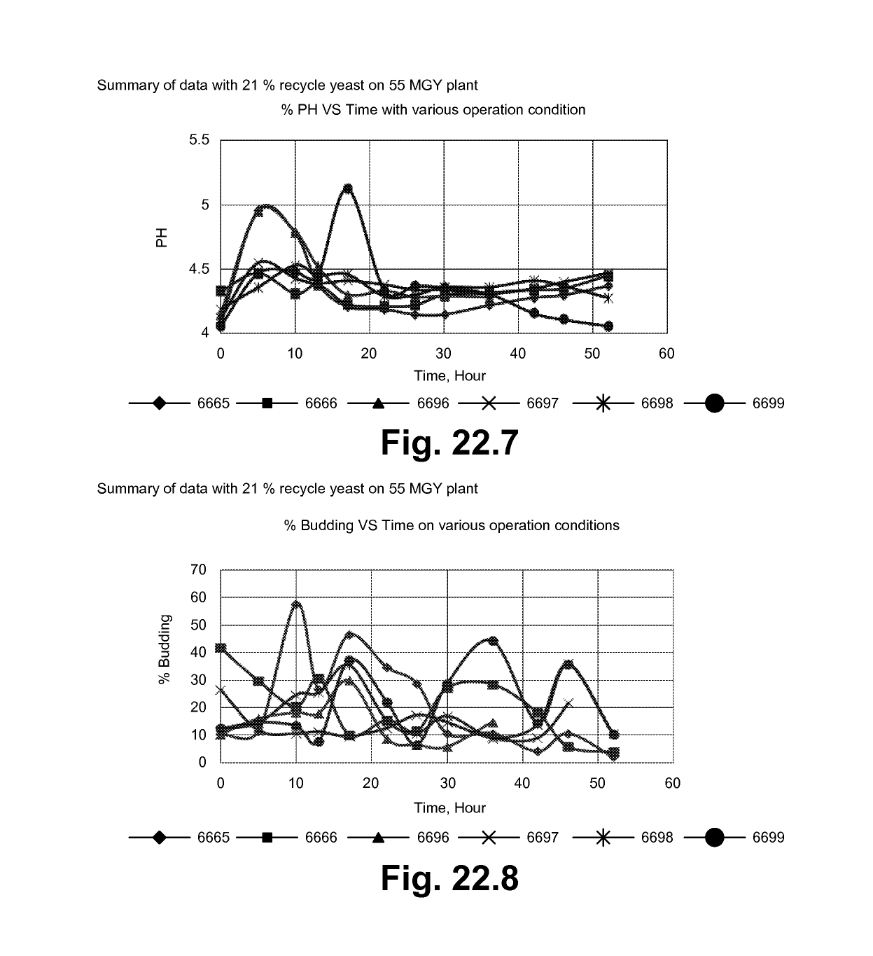

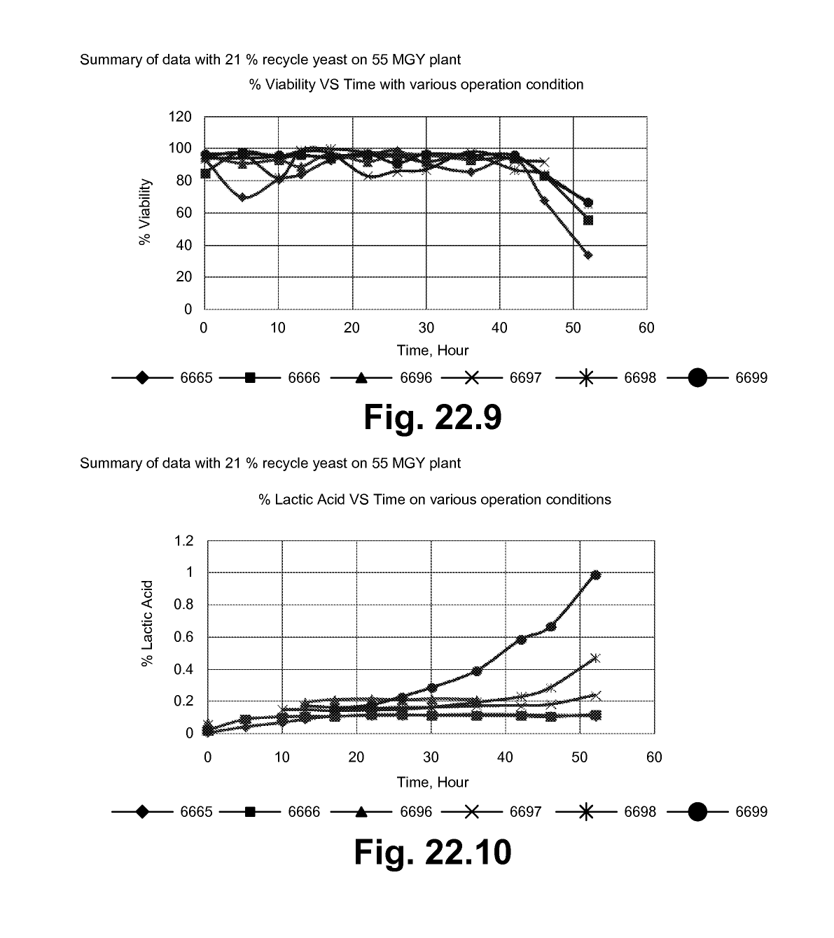

In some embodiments, the constant flow split set up adds one or more control valves for implementation in a production facility. The simplest set up is used to test and compare with the output of a computer simulation. In this setup, a single pulse of material is sent from the aged fermenter to the newly filling fermenter which allows for field application without the introduction of automated control valves. FIG. 21 shows the summary of these test results. FIGS. 22.1-22.10 show the data that are plotted and compared.

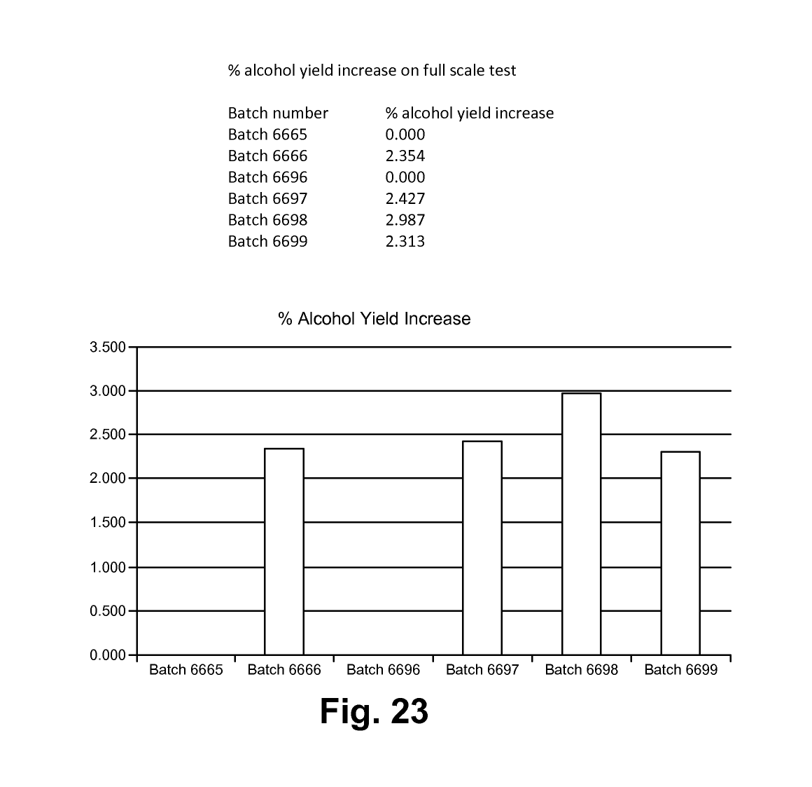

FIG. 23 shows, by comparing the full scale commercial 60 MGY plant with 21% recycle data with 21% recycle computer simulation program output, it can be observed that the computer simulation program represents the commercial operation extremely well. The full scale operation with 21% recycle most active yeast from the set fermenter (aged fermenter) to fill fermenter (newly filling fermenter) gives around 2.4% alcohol yield increase as computer simulation program predicted.

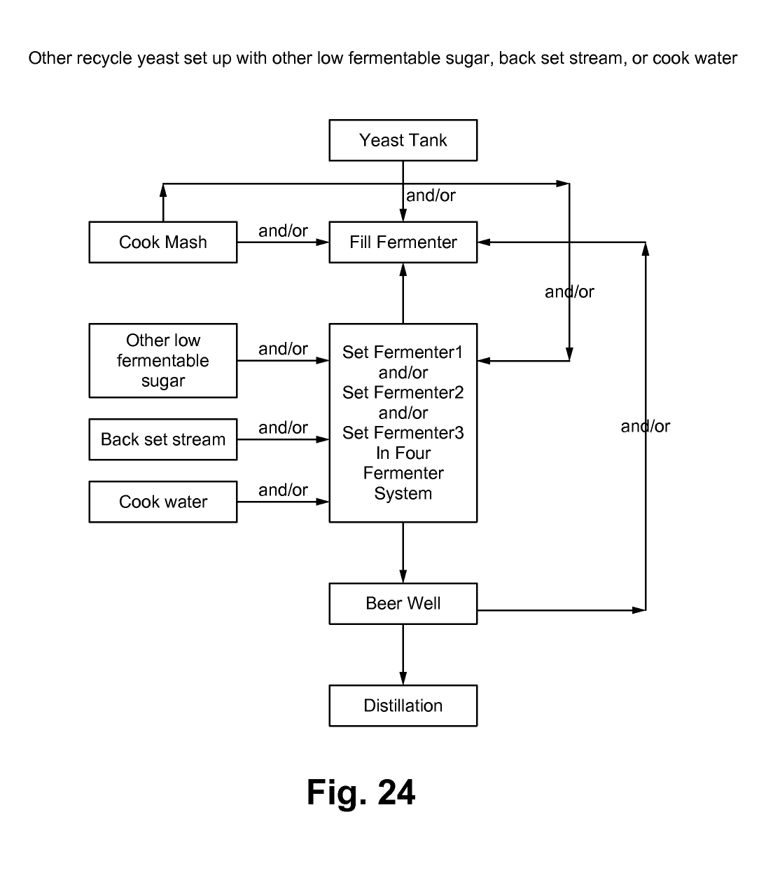

During the initial filling of the fermenter, broth concentrated in yeast can be diverted from any of the previously filled fermenters, beer well and/or yeast tank into the filling fermenter. One of ordinary skill in the art will recognize that the movement of yeast from one, more than one, or all of these tanks into the filling fermenter will increase the concentration of yeast in the filling fermenter resulting in better fermentation of the filling fermenter. As show in FIG. 24, the liquid transferred into the filling fermenter from previously filled (aged) fermenters and/or beer well can be replaced in part or in whole with any number of liquids including, for example: 1) freshly liquefied mash, 2) backset, 3) cook water, 4) other low-fermentable liquids as desired, 5) fermenting broth from another aged fermenter or the freshly filling fermenter or the yeast tank.

In FIGS. 3.3, 5.4, 8.4, 9.4, and 11.3, the % DT and % DT/% Yeast by weight all begin to gradually decrease from hour 1 and reach close to zero around hour 39 then start to increase. It is assumed the % DT will keep increasing at a constant rate because the enzyme exits the system as soon as the liquefied starch. In the actual system, however, the liquefied starch is limited in the actual operation so when the liquefied starch used up by the enzyme, the % DT will not continue increase as shown in all of the plots. The % DT rate increase by GA enzyme is assumed to be constant though all 52 hours in all computer simulation. In the actual operation, this rate is expected to decrease toward end stage in the Fermenter Tank because the liquefied starch becomes depleted and not available to produce additional glucose.

In some embodiments, the improvement system calculations are based on the assumption that the fresh mash sent to the Yeast Tank and the Ferment Tank are constant every hour during the filling period. These rates can be changed every hour to continue the improvement of the result, as one of skill in the art can easily determine. The amount of enzyme in the Yeast tank and Fermenter Tank which are constant in all calculations can also be varied during the operation. The amount of fresh mash and enzyme dosage to the Yeast Tank and Fermenter Tank are varied to insure the Yeast Tank maintains the maximum yeast rate change and create a smooth transfer to Fermenter Tank with minimal shock. Meanwhile, it is important to keep the Fermenter Tank at the high yeast rate change as possible, in the example shown, during the first half of 13 hour filling cycle and allow a smooth transition from yeast growth phase to alcohol production phase on the second half of the 13 hour filling cycle. This will give the optimum result for the fermentation process. Continuous adjustments in the mash rate and enzyme dosage as described to the Yeast Tank and Fermenter Tank plus adjusted process conditions (pH, temperature, nutrition supplements) will give the best fermentation result.

Initially, a little amount of enzyme is required in the Yeast Tank to grow the yeast and keep the DT level low. A higher level of enzyme is required during the fermentation stage because of higher substrate concentration and larger activity of the yeast. Yeast manufacturers have begun to genetically modify yeast to produce GA enzyme during the fermentation stage. This newly engineered yeast has been shown to save around 30% of exogenous GA enzyme addition that is needed for Current System. All of the computer simulations from the improvement systems in this invention have proven that more than double the YCC would be added to the Fermenter Tank. As a result, companies could save more than 60% (2*30) of the GA enzyme when implementing the Low Yeast Tank Improvement System or High Yeast Tank Improvement System. In fact, the Fermenter Tank Improvement may not even need added GA enzyme in Fermenter Tank if practicing this teaching with a GA expressing yeast.

The system improvements mentioned in this patent not only increases the amount of yeast to the Fermenter Tank, but also provides the most active yeast to the Fermenter Tank while minimizing medium shock. This is accomplished by adjusting the % DT/% Yeast by weight while varying fresh mash plus enzyme dosage to switch from a yeast growing phase to alcohol production phase. By this optimizing the whole fermentation step, the data shows the alcohol production starts earlier and is produced faster thus the time in the Fermenter Tank can be shorter. Therefore, a smaller Fermenter Tank or a longer, more complete fermentation cycle will produce more alcohol or a higher concentration of alcohol before distillation. Alternately, higher fermentable solids concentration in the fresh mash could be used while keeping the current fermentation cycle time but increasing the end point concentration of ethanol in the finished fermenter. This may allow plants to increase production rates without adding additional fermentation capacity.

Another benefit of the Low Yeast Tank Improvement System, High Yeast Tank Improvement System, and Fermenter Tank Improvement Systems is that these systems are not capital intensive. The additional material and labor required to switch the typical system to Yeast Tank Improvement System, Fermenter Tank Improvement System, or Combination Tank Improvement System is at a minimal cost. Essentially the only extra tube fittings are needed to creating continuous solution flow to feed in and out of the Yeast Tank or Fermenter Tank.

U.S. Provisional Application No. 60/453,442 (Poet Research, Method for Producing Ethanol Using Raw Starch) filed Apr. 23, 2013, the disclosures of which are hereby incorporated by reference herein in its entirety. Articles titled “Ethanol yield benchmarking at fuel ethanol plants” by Dr. Dennis Bayrock, R&D Phibro Ethanol Performance Group, and articles titled “Modeling of the Kinetic from Glucose Biomass in Batch Culture with Non Structured Model” by Olaoye O.S. and Kolawole O.S. and “Controlling Glucose Levels in Fermentation for Optimal Yeast Performance” by Nick LeFebvr are also hereby incorporated by reference herein in their entirety for all purposes.

Some examples of the present invention:

In an aspect, a method of maintaining a higher yeast concentration and more active yeast in a production fermenter is disclosed. The method comprises modifying yeast propagation practice in yeast growth tank by adding yeast into the production fermenter during a filling period. The yeast propagator is able to be started earlier thereby allowing longer incubation time of yeast before the yeast is transferred to a production fermenter that are filled with mash. In some embodiments, the yeast propagator is operated with a higher aeration than traditionally found in fuel ethanol facilities to produce higher aerobic respiration potential resulting in increased cell densities before transfer to production fermenter.

In some embodiments, the yeast propagator is operated with lower % DS broth than the production fermenter resulting in increased cell densities before it is transferred to a production fermenter. In some embodiments, the yeast propagator is operated with augmented nutritional factors which are able to include formulated yeast foods, endo proteases, exo proteases, combination of endo and exo proteases, higher additions of assimilable nitrogen resulting in increased cell densities before transfer to production fermenter.

In another aspect, a method of maintaining a higher yeast concentration and more active yeast in a production fermenter by transferring a volume of fermenter broth containing yeast from previously set production fermenter(s) and/or beerwell to the filling production fermenter before or during the filling period is disclosed. In some embodiments, a yeast propagator is not used to transfer yeast into the filling production fermenter.

In some embodiments, the glucoamylase from the previously set production fermenter(s) and/or beerwell in the broth reduces the fresh glucoamylase dose required for the filling fermenter. In some embodiments, the % glucose is maintained below 4% during the filling period reducing yeast stress and reducing glycerol production. In some embodiments, shock to incoming yeast is minimized in the fermenter during the filling and/or fermentation period. In some embodiments, % glucose is maintained below 4% during the entire fermentation step for the filling fermenter. In some embodiments, the total fermentation time is reduced 1 to 14 hours.

In some embodiments, the total fermentation time is reduced 3 to 12 hours. In some embodiments, the total fermentation time is reduced 4 to 10 hours. In some embodiments, the total fermentation time is reduced 6 to 8 hours. In some embodiments, the fermenter has lower % glycerol production due to reduced osmotic stress from high glucose concentration. In some embodiments, the parameter, % DT/% Yeast by weight, is used as a control factor to maintain the most active yeast condition in both the propagation tank(s) and the production fermenter tank(s).

In some embodiments, the fresh mash is transferred to both a filling production fermenter and already set production fermenter(s) such that the each volume of fresh mash transferred to the set production fermenter(s) is accompanied by a volume from that set production fermenter(s) being transferred to the filling fermenter. In some embodiments, fresh mash is transferred to filling production fermenter(s) and/or beerwell and broth containing yeast from already set production fermenter(s) and/or beerwell is also transferred to the filling production fermenter. In some embodiments, a volume of broth containing yeast from already set production fermenter(s) or beerwell is transferred to an empty production fermenter prior to filling that empty production fermenter with fresh mash.

In some embodiments, fresh mash is transferred to both a filling production fermenter and one or more already set production fermenter(s) and/or beerwell while also transferring a volume of fermenting broth from an already set production fermenter(s) and/or beerwell to the filling production fermenter. In some embodiments, a low-fermentable liquid, such as, for example backset, cook water, CO₂ scrubbing water, or fresh water, is transferred to one or more already set production fermenter(s) and/or beerwell diluting the fermention broth.

A portion, up to all, of the fermenting broth displaced from the one or more set production fermenter(s) or beerwell is transferred to a filling production fermenter before, during or after filling process. In some embodiments, a low-fermentable liquid, such as, for example backset, cook water, CO₂ scrubbing water, or fresh water, along with fresh mash in any proportion is transferred to one or more already set production fermenter(s) and/or beerwell diluting the fermenting broth.

A portion, up to all, of the fermention broth displaced from the one or more set production fermenter(s) and/or beerwell is transferred to at least the filling production fermenter either before, during or after transfer of mash to the filling production fermenter. In some embodiments, the fermentable solids concentration is raised in fermentation and the fermenter finishes fermentation at higher alcohol concentration without increasing the overall fermentation time.

In another aspect, a method to extend active yeast growth time in a filled production fermenter by transferring a volume of fermenting broth from current filled production fermenter and replacing at least some of the transferred volume with liquid composed of unfermented mash and/or low-fermentable liquid in any proportion is provided. In some embodiments, a portion of fermenting broth is transferred from current set production fermenter and a volume of liquid is transferred to the set production fermenter resulting in at least temporary dilution of the fermenter broth ethanol concentration.

In some embodiments, a portion of fermenting broth is transferred from current set production fermenter and a volume of liquid is transferred to the set production fermenter resulting in at least temporary dilution of the fermenter broth ethanol concentration, wherein the volume of liquid transferred to the set production fermenter is a mixture of fresh mash and low-fermentable liquid in any proportion.

The meaning of the following terms include: 1) fresh mash: starchy grain slurry that has not been inoculated with a viable yeast culture for the purpose of inducing fermentation, 2) low-fermentable liquid: liquid that contains lower starch concentration than fresh mash, 3) grain: any grain type that contains at least 10% starch on a dry matter basis. (Non-exhaustive examples include: grain, maize, wheat, sorghum, barley, oats and triticale), 4) grain fractions: parts of grain or ground grain that have had some portions selectively removed thereby enriching or depleting the resulting material in starch concentration compared to the whole source grain, 5) grain carbohydrate: any mixture proportion of grain and grain fractions including different types (species) of grains and different types (species) of grain fractions, 6) DT: dextrose, 7) % DT: units of dextrose by mass per 100 units of total material, 8) YCC: yeast cell count, 9) GA: glucoamylase enzyme, and 10) Yeast Tank: special fermentation tank, generally smaller than the production fermenters and designed specifically to encourage yeast growth, often called a yeast propagator or yeast conditioning tank.

Yeast growth improvement in the Yeast Tank and Fermenter Tank methods include (1) using different types of yeast, (2) adjusting the pH, (3) changing the temperature, (4) adding nitrogen for nutrition, (5) adding zinc, (6) adding air, (7) adding formulated nutrient packages. These techniques have been proven to provide some yeast growth improvement.

In operation, the present invention includes 1) continuing to produce the most active yeast either from the Yeast Tank or an Aged Fermenter Tank during the filling period, 2) adjusting the amount of fresh mash into the Yeast Tank and/or Fermenter Tank on the most active state and produce more yeast, 3) using a controlling parameter % DT/% Yeast by weight as a guideline for maintaining yeast in the most active condition, 4) adjusting the amount of fresh mash and enzyme dosage to the Fermenter Tank during the filling period so that it does not create shock to the system, and 5) maintaining a smooth transition from the most active yeast state into producing alcohol state during the filling period.

In utilization, the present invention is used to grow higher and stronger yeast in the yeast tank and fermenter tank during a filling cycle.

The present invention has been described in terms of specific embodiments incorporating details to facilitate the understanding of principles of construction and operation of the invention. Such reference herein to specific embodiments and details thereof is not intended to limit the scope of the claims appended hereto. It is readily apparent to one skilled in the art that other various modifications can be made in the embodiment chosen for illustration without departing from the spirit and scope of the invention as defined by the claims.

What is claimed is:

1. A fermentation method comprising:
a) adjusting a health condition of a yeast in a yeast solution based on a ratio of % DT/% Yeast by weight; and
b) continuously inputting the yeast solution in a fermenter tank during a filling period.

2. The method of claim 1, wherein the ratio of the % DT/% Yeast by weight is adjusted to optimize the health condition of the yeast condition.

3. The method of claim 1, wherein the health condition comprises an active condition of the yeast in the yeast solution.

4. The method of claim 1, further comprising adjusting the ratio of % DT/% Yeast by weight, such that the fermenter tank generates less glycerin.

5. The method of claim 1, further comprising preventing the ratio of the % DT/% Yeast by weight exceeding a higher threshold to prevent a stress of the yeast.

6. The method of claim 1, further comprising preventing the ratio of the % DT/% Yeast by weight prematurely below a lower threshold to prevent a death of the yeast.

7. The method of claim 1, wherein the ratio of the % DT/% Yeast by weight is adjusted based on a sugar and the yeast concentration.

8. The method of claim 1, wherein the ratio of the % DT/% Yeast by weight is adjusted based on a sugar flow rate, an enzyme dosage, a pH value, and a temperature of the fermenter tank.

9. A fermentation method comprising:
a) providing a continuous flow of a yeast solution in a fermenter tank during a filling period;
b) monitoring a ratio of % DT/% Yeast by weight; and
c) adjusting a rate of the continuous flow based on the ratio.

10. The method of claim 9, wherein the continuous flow of a yeast solution is from a yeast tank.

11. The method of claim 10, further comprising sending a first volume of a fresh mash feed to the yeast tank.

12. The method of claim 11, further comprising sending a second volume of the yeast solution from the yeast tank to the fermenter tank, wherein the second volume is substantially the same as the first volume.

13. A method of fermentation tank improvement comprising:
a) providing a first amount of a sugar solution to a first fermenter tank and a second amount of the sugar solution to a second fermenter tank;
b) providing a third amount of a fermenting solution from the second fermenter tank to the first fermenter tank, wherein the third amount is substantially equal to the second amount.

14. The method of claim 13 further comprising sending a fourth amount of yeast solution from a yeast tank to the first fermenter tank.

15. The method of claim 13, wherein the first fermenter tank is a new fermenter tank and the second fermenter tank is an aged fermenter tank at a first time period.

16. The method of claim 15, wherein the first fermenter tank becomes an aged fermenter tank providing a fifth amount of a fermenting solution from the first fermenter tank to a third fermenter tank at a second time period.

17. The method of claim 16, wherein the first fermenter tank receives the firth amount of a sugar solution.

18. The method of claim 17, wherein the third fermenter tank is a new fermenter tank and receives the yeast solution from the yeast tank.

19. A method of fermentation tank and yeast tank improvement comprising:
a) providing a first amount of a fresh mash feed to a yeast tank;
b) providing a second amount of the fresh mash feed to an aged fermenter tank;
c) providing the first amount of a yeast solution from the yeast tank to a first filling fermenter tank; and
d) providing the second amount of a first fermenting solution from the aged fermenter tank to the first filling fermenter tank.

20. The method of 19, wherein the first filling fermenter tank is used as a second aged fermenter tank at a next time period.

21. The method of 20, wherein the second aged fermenter tank is used to provide a second fermenting solution to a second filling fermenter tank.

22. The method of claim 19, wherein a concentration of a yeast of the yeast solution added to the first filling fermenter tank is elevated due to yeast addition from the aged fermenter leading to a faster fermentation.

23. The method of claim 19, further comprising a time for a fermentation progress to reach endpoint is reduced by 1 to 14 hours.

24. The method of claim 19, further comprising a time for a fermentation progress to reach endpoint is reduced by 6 to 8 hours.

25. The method of claim 19, further comprising not using a micro-aerated yeast propagator to condition yeast for addition of yeast into the filling fermenter.

26. The method of claim 19, wherein the mash from the aged fermenter provided to the first filling fermenter tank results in the reduction of the addition of freshly purchased glucoamylase enzyme to the first filling fermenter tank.

27. The method of claim 19, further comprising maintaining a glucose concentration below 4% during a filling period of the first filling fermenter to reduce yeast stress and reduce glycerol production.

28. The method of claim 19, further comprising maintaining a glucose concentration below 4% during the entire fermentation for the first filling fermenter.

29. The method of claim 19, wherein the fresh mash is transferred to both a first filling fermenter and one or more already set aged fermenters such that each volume of the fresh mash transferred to the aged fermenter is accompanied by a volume from the aged fermenter being transferred to the first filling fermenter.

30. The method of claim 19, further comprising transferring a volume of fermentation broth containing yeast from at least one aged fermenter or beerwell to an empty fermenter prior to adding the fresh mash to the first filling fermenter.

31. The method of claim 19, further comprising transferring the fresh mash to both a first filling fermenter and one or more aged fermenter or beerwell while also transferring a volume of fermenting broth from one or more aged fermenters or beerwell to the first filling fermenter.

32. The method of claim 19, further comprising transferring a low-fermentable liquid including a backset, cook water, CO₂ scrubbing water, or fresh water to one or more aged production fermenters or beerwell diluting the fermentation broth.

33. The method of claim 19, further comprising transferring a low-fermentable liquid including a backset, cook water, CO₂ scrubbing water, or fresh water, along with the fresh mash transferred to one or more aged fermenters or beerwell diluting the fermenting broth.

34. The method of claim 19, further comprising raising a fermentable solids concentration in fermentation.

35. A method of enhancing fermentation process comprising:
a) selectively taking a concentrated yeast slurry containing a CO₂ froth layer at a top portion of an aged fermenter; and
b) adding the yeast slurry to a new fermenter.

36. The method of claim 35, wherein the CO₂ froth layer comprises a portion of higher active yeast than the remaining yeasts in a yeast tank.

37. The method of claim 35, wherein the concentrated yeast slurry is in an overflow stream from the yeast tank.

38. A fermentation system comprising:
a) multiple fermentation tanks including an aged tank and a young tank; and
b) a yeast tank, wherein the yeast tank provides a yeast solution and the aged tank provide a fermenting solution to the young tank.

39. The system of claim 38, further comprising a rotating mechanism rotating feed to the multiple fermentation tanks when the young tank is full.

40. The system of claim 38, further comprising a sugar solution providing source providing a sugar solution to both the young tank and the aged tank.

41. A method of extending active yeast growth time in a filled, aging fermenter comprising:
transferring a volume of a fermenting broth from a currently filled production fermenter; and
replacing at least some of the transferred volume with a liquid comprising an unfermented mash or low-fermentable liquid.

42. The method of claim 41, wherein a portion of the fermenting broth is transferred from an aging production fermenter and a volume of the liquid is transferred to another aging fermenter resulting in at least temporary dilution of an ethanol concentration of the fermenter broth.

43. The method of claim 41, wherein a portion of fermenting broth is transferred from the currently aging fermenter and a volume of liquid is transferred to another aging fermenter resulting in at least temporary dilution of a ethanol concentration of the fermenter broth.

44. A method of maintaining a higher yeast concentration and more active yeast in a fermenter comprising:
modifying a yeast propagation practice in a yeast growth tank; and
adding yeast into a first young filling fermenter during a filling period.

45. The method of claim 44, wherein a yeast propagator of the yeast propagation practice is operated at an aeration rate producing an aerobic respiration rate resulting in an increased cell densities before the yeast is transferred to a first young filling fermenter.

46. The method of claim 44, wherein the yeast propagator is operated with a lower % DS broth than a production fermenter resulting in an increased cell densities before transferring to a first young filling fermenter.

48. The method of claim 44, wherein the yeast propagator is operated with augmented nutritional factors including formulated yeast foods, endo proteases, exo proteases, combination of endo and exo proteases, assimilable nitrogens resulting in an increased cell densities before transferring to a first young filling fermenter.

49. A method of starting a fermenter with a substantially high yeast concentration and active yeast in a batch fermentation plant comprising:
transferring mash containing yeast from a previously filled fermenter or beerwell to a newly filling fermenter.

50. The method of claim 49, wherein a yeast propagator is not used to introduce yeast into the newly filling fermenter.

51. The method of claim 50, wherein an amount of yeast metabolizable oxygen is supplied to the newly filling fermenter.

52. The method of claim 50, wherein no supplemental metabolizable oxygen is supplied to the newly filling fermenter.

53. The method of claim 49, wherein a yeast propagator is used to introduce conditioned yeast into the newly filling fermenter.

54. The method of claim 49, wherein an amount of the mash transferred from a previously filled fermenter or beerwell is between 0 and 50% of the volume of the newly filling fermenter.

55. The method of claim 49, wherein an amount of the mash transferred from a previously filled fermenter or beerwell is between 10 and 40% of the volume of the newly filling fermenter.

56. The method of claim 49, wherein an amount of the mash transferred from a previously filled fermenter or beerwell is between 20 and 30% of the volume of the newly filling fermenter.

57. The method of claim 49, wherein an amount of the mash transferred from a previously filled fermenter or beerwell is between 21 and 25% of the volume of the newly filling fermenter.

58. The method of claim 49, wherein an amount of the mash transferred from a previously filled fermenter or beerwell is added before fresh mash is added to the newly filling fermenter.

59. The method of claim 49, wherein an amount of the mash transferred from a previously filled fermenter or beerwell is added simultaneously with a fresh mash adding to the newly filling fermenter.

60. The method of claim 49, wherein an amount of the mash transferred from a previously filled fermenter or beerwell is added after a fresh mash has been added to the newly filling fermenter.

61. The method of claim 49, wherein an amount of yeast metabolizable oxygen is supplied to the newly filling fermenter.

62. The method of claim 61, wherein air is a source of metabolizable oxygen supplied to the newly filling fermenter.

63. The method of claim 61, wherein a mash soluble chemical is a source of oxygen supplied to the newly filling fermenter, such as, for example, hydrogen peroxide.

64. The method of claim 61, wherein diatomic molecular oxygen (O₂) is the source of metabolizable oxygen supplied to the newly filling fermenter.

65. The method of claim 49, wherein no supplemental metabolizable oxygen is supplied to the newly filling fermenter.
